(19) United States
(12) Patent Application Publication
(10) Pub. No.: US 2026/0174905 A1
(43) Pub. Date: Jun. 25, 2026

(54) COMPOSITIONS AND METHODS FOR IMPROVED ADENOVIRAL-BASED GENE THERAPY UTILIZING CORTICOSTEROID TREATMENT

(71) Applicants: Pacira Therapeutics, Inc., Brisbane, CA (US); Pacira Germany GmbH, Hamburg (DE)

(72) Inventors: Roy Winston, Tampa, FL (US); Ronald J. Ellis, JR., Tampa, FL (US); Kilian Guse, Berlin (DE); Stanislav Plutizki, Hamburg (DE); Manish Solanki, Kleinmachnow (DE)

(21) Appl. No.: 19/314,811

(22) Filed: Aug. 29, 2025

Related U.S. Application Data
(63) Continuation of application No. PCT/US2024/017743, filed on Feb. 28, 2024.
(60) Provisional application No. 63/488,130, filed on Mar. 2, 2023.

(51) Int. Cl. A61K 48/00; A61K 9/00; A61K 31/573; A61K 38/17; A61P 19/02; C12N 15/86

(52) U.S. Cl. CPC A61K 48/0058; A61K 9/0019; A61K 31/573; A61K 38/1793; A61K 48/0075; A61K 48/0083; A61P 19/02; C12N 15/86; C12N 2750/14143

(57) ABSTRACT

The present disclosure relates to pharmaceutical compositions, kits, methods and uses thereof, that include a corticosteroid and an adenoviral-based delivery and expression system. In some embodiments, the compositions, kits, methods and uses thereof, improve one or more of transduction, expression, efficacy and/or safety of the adenoviral-based delivery and expression system when administered to an animal as compared to administration of the same pharmaceutical composition without the corticosteroid.

### INCORPORATION-BY-REFERENCE OF SEQUENCE LISTING

The instant application contains a Sequence Listing which has been submitted in XML format via EFS-Web and is hereby incorporated by reference in its entirety. Said XML copy, created on Mar. 9, 2026, is named “PCRTX_018C1” and is 203,453 bytes in size.

### CROSS-REFERENCE TO RELATED APPLICATIONS

This application is continuation of International Patent Application No. PCT/US2024/017743, filed on Feb. 28, 2024, which claims priority to the U.S. Provisional Patent Application Ser. No. 63/488,130, filed on Mar. 2, 2023, each of which is incorporated by reference herein in its entirety.

### BACKGROUND OF THE DISCLOSURE

The use of adenoviral vectors for gene therapy is increasing. Both adenovirus and helper-dependent adenoviral vectors (HDAd) are being explored for use in gene therapy. HDAd have genomes that are devoid of all viral coding sequences, and contain only the inverted terminal repeats (ITR) required for vector genome replication, and the adenoviral packaging signal (Ψ) necessary for packaging for the viral genome into the capsid produced by the helper adenovirus. Because of the lack of viral coding sequences, HDAd have a larger cloning capacity, and are thought to illicit a weaker host immune response, than adenoviral vectors. However, HDAd can be more difficult to manufacture than adenoviral vectors. Despite advances in the use of adenoviral based vectors, there remains a need for improving the usefulness of both adenoviral and HDAd vectors for gene therapy.

### SUMMARY OF THE DISCLOSURE

The present disclosure relates to pharmaceutical compositions, kits, methods and uses thereof, that include a corticosteroid and an adenoviral-based delivery and expression system. In some embodiments, the compositions, kits, methods and uses thereof, improve one or more of transduction, expression, efficacy and/or safety of the adenoviral-based delivery and expression system when administered to an animal as compared to administration of the same pharmaceutical composition without the corticosteroid.

Embodiments of the present disclosure include the following numbered embodiments:
1. A pharmaceutical composition comprising a corticosteroid and an adenoviral-based delivery and expression system.
2. The pharmaceutical composition of embodiment 1, wherein the corticosteroid is a glucocorticoid, a mineralocorticoid, or both.
3. The pharmaceutical composition of embodiment 1 or 2, wherein the corticosteroid is selected from the group consisting of:
   beclomethasone, betamethasone, budesonide, cortisone, deflazacort, dexamethasone, hydrocortisone, methylprednisolone, prednisolone, prednisone, triamcinolone, fludrocortisone, deoxycorticosterone, aldosterone, ciclesonide, fluticasone, flunisolide, mometasone, and mixtures thereof;
   beclomethasone, betamethasone, budesonide, cortisone, deflazacort, dexamethasone, hydrocortisone, methylprednisolone, prednisolone, prednisone, triamcinolone, fludrocortisone, deoxycorticosterone, aldosterone, and mixtures thereof;
   beclomethasone, betamethasone, budesonide, cortisone, dexamethasone, hydrocortisone, methylprednisolone, prednisolone, prednisone, triamcinolone, and mixtures thereof;
   beclomethasone, betamethasone, budesonide, cortisone, dexamethasone, hydrocortisone, methylprednisolone, prednisolone, triamcinolone, and mixtures thereof;
   betamethasone, dexamethasone, triamcinolone, methylprednisolone, and mixtures thereof;
   beclomethasone, budesonide, ciclesonide, fluticasone, flunisolide, mometasone, and mixtures thereof; and/or
   fludrocortisone, deoxycorticosterone, aldosterone, and mixtures thereof.
4. The pharmaceutical composition of any one of embodiments 1-3, wherein the corticosteroid is selected from the group consisting of: betamethasone, dexamethasone, triamcinolone, methylprednisolone, and mixtures thereof; optionally wherein the corticosteroid is methylprednisolone; optionally wherein the corticosteroid is methylprednisolone acetate.
5. The pharmaceutical composition of any one of embodiments 1-4, wherein the adenoviral-based delivery and expression system comprises a nucleic acid encoding a protein for expression by the adenoviral-based delivery and expression system; optionally wherein the protein is a therapeutic protein.
6. The pharmaceutical composition of any one of embodiments 1-5, wherein the adenoviral-based delivery and expression system is a recombinant adenoviral vector or a helper-dependent adenoviral vector, optionally an Ad5 serotype.
7. The pharmaceutical composition of embodiment 6, wherein the helper-dependent adenoviral vector comprises a genome comprising or consisting of: the nucleic acid sequence encoding the protein; a promoter regulating expression of the nucleic acid sequence encoding the protein; left and right inverted terminal repeats; an adenoviral packaging signal; and non-viral and non-coding stuffer nucleic acid sequences, optionally wherein the promoter is located upstream of the reading frame of the nucleic acid sequence encoding the protein such that the expression of protein is regulated by the promoter.
8. The pharmaceutical composition of embodiment 7, wherein the promoter is selected from:
   a ubiquitous, constitutive promoter; optionally selected from the group consisting of elongation factor 1 alpha (EF1 alpha) promoter, cytomegalovirus (CMV) promoter, beta-actin promoter, simian virus 40 (SV40) early promoter, ubiquitin c promoter, glyceraldehyde 3-phosphate dehydrogenase (GAPDH) promoter, and phosphoglycerate kinase (PGK) promoter; or
   inflammation-sensitive promoter; optionally selected from the group consisting of a promoter inducible by NF-κB, interleukin 6 (II-6), interleukin-1 (IL-1), tumor necrosis factor (TNF), cyclooxygenase 2 (COX-2), complement factor 3 (C3), serum amyloid A3 (SAA3), and macrophage inflammatory protein-1a (MIP-1a).
9. The pharmaceutical composition of embodiment 7 or 8, wherein the genome comprises or consists of a nucleic acid that is, or is at least, 80%, 85%, 90%, 95%, 96%, 97%, 98%, 99%, or 100% identical to SEQ ID NO: 7 or 8, excluding the promoter and the nucleic acid sequence encoding the protein.
10. The pharmaceutical composition of any one of embodiments 5-9, wherein the nucleic acid encoding the protein encodes for:
   interleukin-1 receptor antagonist (IL-1Ra), proteoglycan 4 (PRG4), or both IL-1Ra and PRG4, or a biologically active fragment of at least one of the preceding;
   IL-1Ra, PRG4, or both IL-1Ra and PRG4;
   IL-1Ra; or
   PRG4.
11. The pharmaceutical composition of any one of embodiments 5-10, wherein the nucleic acid encodes for:
   a mammalian protein;
   an equine, canine, feline, or murine protein; or
   a human protein;
      optionally wherein the nucleic acid is codon optimized.
12. The pharmaceutical composition of any one of embodiments 5-11, wherein the nucleic acid encoding the protein comprises or consists of a nucleic acid that is, or is at least, 80%, 85%, 90%, 95%, 96%, 97%, 98%, 99%, or 100% identical or homologous to a sequence selected from SEQ ID NOs: 1, 4, 5, 10, 11, 14, 17, 18 or 19; optionally wherein the sequence is codon optimized.
13. The pharmaceutical composition of any one of embodiments 7-12, wherein the genome comprises or consists of a nucleic acid that is, or is at least, 80%, 85%, 90%, 95%, 96%, 97%, 98%, 99%, or 100% identical to SEQ ID NOs: 2, 3, 7, 8, 9.
14. The pharmaceutical composition of any one of embodiments 7-12, wherein the nucleic acid encodes a PRG4 protein comprising or consisting of an amino acid sequence that is, or is at least, 80%, 85%, 90%, 95%, 96%, 97%, 98%, 99%, or 100% identical or homologous to a sequence selected from SEQ ID NO: 12, 13, 17, 18, or 19, or a biologically active fragment thereof, or a homolog thereof from any other species.
15. The pharmaceutical composition of any one of embodiments 7-12, wherein the nucleic acid encodes an IL-1Ra protein comprising or consisting of an amino acid sequence that is, or is at least, 80%, 85%, 90%, 95%, 96%, 97%, 98%, 99%, or 100% identical or homologous to a sequence selected from SEQ ID NO: 6, 15 or 16, or a biologically active fragment thereof, or a homolog thereof from any other species.
16. The pharmaceutical composition of any one of embodiments 7-14, wherein the nucleic acid encodes for PRG4, and expression of PRG4 is regulated by a ubiquitous, constitutive promoter; optionally selected from the group consisting of elongation factor 1 alpha (EF1 alpha) promoter, cytomegalovirus (CMV) promoter, beta-actin promoter, simian virus 40 (SV40) early promoter, ubiquitin c promoter, glyceraldehyde 3-phosphate dehydrogenase (GAPDH) promoter, and phosphoglycerate kinase (PGK) promoter.
17. The pharmaceutical composition of any one of embodiments 7-13 or 15, wherein the nucleic acid encodes for IL-1Ra, and expression of IL-1Ra is regulated by an inflammation-sensitive promoter; optionally wherein the inflammation-sensitive promoter is selected from the group consisting of a promoter inducible by NF-κB, interleukin 6 (II-6), interleukin-1 (IL-1), tumor necrosis factor (TNF), cyclooxygenase 2 (COX-2), complement factor 3 (C3), serum amyloid A3 (SAA3), and macrophage inflammatory protein-1a (MIP-1a).
18. The pharmaceutical composition of any one of embodiments 7-17, wherein the promoter is located upstream of the reading frame of the nucleic acid sequence encoding the protein, such that the expression of protein is regulated by the promoter.
19. The pharmaceutical composition of any one of embodiments 1-18, wherein the adenoviral-based biological delivery and expression system comprises a helper-dependent adenoviral vector comprising a nucleic acid sequence encoding animal IL-1Ra protein, left and right inverted terminal repeats, an adenoviral packaging signal and non-viral, and non-coding stuffer nucleic acid sequences, wherein the expression of the nucleic acid sequence encoding animal IL-1Ra protein is regulated by a NF-κB inducible promoter, which is located upstream of the reading frame of the nucleic acid sequence encoding the animal IL-1Ra protein, optionally wherein the animal is equine, canine, feline or murine.
20. The pharmaceutical composition of any one of embodiments 1-18, wherein the adenoviral-based biological delivery and expression system comprises a helper-dependent adenoviral vector comprising a nucleic acid sequence encoding human IL-1Ra protein, left and right inverted terminal repeats, an adenoviral packaging signal and non-viral, and non-coding stuffer nucleic acid sequences, wherein the expression of the nucleic acid sequence encoding human IL-1Ra protein is regulated by a NF-κB inducible promoter, which is located upstream of the reading frame of the nucleic acid sequence encoding the human IL-1Ra protein.
21. The pharmaceutical composition of any one of embodiments 1-18, wherein the adenoviral-based biological delivery and expression system comprises a helper-dependent adenoviral vector comprising a nucleic acid sequence encoding animal PRG4 protein, left and right inverted terminal repeats, an adenoviral packaging signal and non-viral, and non-coding stuffer nucleic acid sequences, wherein the expression of the nucleic acid sequence encoding animal PRG4 protein is regulated by a EF1 alpha inducible promoter, which is located upstream of the reading frame of the nucleic acid sequence encoding the animal PRG4 protein, optionally wherein the animal is equine, canine, feline or murine.
22. The pharmaceutical composition of any one of embodiments 1-18, wherein the adenoviral-based biological delivery and expression system comprises a helper-dependent adenoviral vector comprising a nucleic acid sequence encoding human PRG4 protein, left and right inverted terminal repeats, an adenoviral packaging signal and non-viral, and non-coding stuffer nucleic acid sequences, wherein the expression of the nucleic acid sequence encoding human PRG4 protein is regulated by a EF1 alpha inducible promoter, which is located upstream of the reading frame of the nucleic acid sequence encoding the human PRG4 protein.
23. The pharmaceutical composition of any one of embodiments 1-22, wherein the pharmaceutical composition is formulated for localized administration; optionally for administration topically, by injection, by inhalation, intra-ocularly, or intra-ear.
24. The pharmaceutical composition of any one of embodiments 1-23, wherein the pharmaceutical composition is formulated for injection into a joint; optionally for intra-tendinous, intra-muscular, intra-articular, or sub-acromial injection.
25. The pharmaceutical composition of any one of embodiments 1-24, wherein the pharmaceutical composition is formulated for intra-articular injection into a human joint; optionally wherein the joint is the knee.
26. The pharmaceutical composition of any one of embodiments 1-23, wherein the pharmaceutical composition is formulated for injection into an intervertebral disc; optionally wherein the injection is intradiscal injection, optionally into the nucleus pulposus (NP) region of the intervertebral disc.
27. The pharmaceutical composition of any one of embodiments 1-23, wherein the pharmaceutical composition is not formulated for administration into an intervertebral disc; optionally wherein the administration is intradiscal injection, optionally into the nucleus pulposus (NP) or central gelatinous nucleus pulposus (NP) region of the intervertebral disc.
28. The pharmaceutical composition of any one of embodiments 1-27, wherein the pharmaceutical composition is a liquid.
29. The pharmaceutical composition of any one of embodiments 1-28, wherein the adenoviral-based delivery and expression system is in a buffer comprising TRIS 10 mM, NaCl 75 mM, Polysorbate 80 0.02% (v/v), sucrose 5% (w/v), MgCl₂ 1.0 mM, EDTA 100 μM, ethanol 0.5% (v/v), and L-histidine 10 mM.
30. The pharmaceutical composition of any one of embodiments 1-29, wherein the adenoviral-based delivery and expression system is a recombinant adenoviral vector or a helper-dependent adenoviral vector, and the concentration of vector is, is at least, or is not more than, 1.4×10⁵, 1.4×10⁶, 2.8×10⁶, 7.0×10⁶, 1.4×10⁷, 2.8×10⁷, 7.0×10⁷, 1.4×10⁸, 2.8×10⁸, 7.0×10⁸, 1.4×10⁹, 2.8×10⁹, 7.0×10⁹, 1.4×10¹⁰, 2.8×10¹⁰, 7.0×10¹⁰, 1.4×10¹¹, 2.8×10¹¹, 7.0×10¹¹, 1.4×10¹², 2.8×10¹², 7.0×10¹², 1.4×10¹³, 2.8×10¹³, 7.0×10¹³ or a range defined by any two of the preceding values, optionally 1.4×10⁵ to 7.0×10¹³, 1.4×10⁵ to 7.0×10¹², 1.4×10⁵ to 7.0×10¹¹, 1.4×10⁵ to 7.0×10¹⁰, 1.4×10⁵ to 7.0×10⁹, 1.4×10⁵ to 7.0×10⁸, 1.4×10⁹ to 7.0×10¹⁰, optionally 2.8×10⁹, 2.8×10¹⁰, 2.8×10¹¹, genome copies (GC) of recombinant adenoviral vector or a helper-dependent adenoviral vector per ml of the pharmaceutical composition.
31. The pharmaceutical composition of any one of embodiments 1-30, wherein the adenoviral-based delivery and expression system is a recombinant adenoviral vector or a helper-dependent adenoviral vector, and the concentration of vector is, is at least, or is not more than, 1.4×10⁵, 1.4×10⁶, 2.8×10⁶, 7.0×10⁶, 1.4×10⁷, 2.8×10⁷, 7.0×10⁷, 1.4×10⁸, 2.8×10⁸, 7.0×10⁸, 1.4×10⁹, 2.8×10⁹, 7.0×10⁹, 1.4×10¹⁰, 2.8×10¹⁰, 7.0×10¹⁰, 1.4×10¹¹, 2.8×10¹¹, 7.0×10¹¹, 1.4×10¹², 2.8×10¹², 7.0×10¹², 1.4×10¹³, 2.8×10¹³, 7.0×10¹³ or a range defined by any two of the preceding values, optionally 1.4×10⁵ to 7.0×10¹³, 1.4×10⁵ to 7.0×10¹², 1.4×10⁵ to 7.0×10¹¹, 1.4×10⁵ to 7.0×10¹⁰, 1.4×10⁵ to 7.0×10⁹, 1.4×10⁵ to 7.0×10⁸, 1.4×10⁹ to 7.0×10¹⁰, viral particles (VP) of recombinant adenoviral vector or a helper-dependent adenoviral vector per ml of the pharmaceutical composition.
32. The pharmaceutical composition of any one of embodiments 1-31, wherein the pharmaceutical composition is a liquid having a volume that is, is at least, or is not more than, 0.1, 0.25, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10 ml, or a range defined by any two of the preceding values, optionally 0.1-10, 0.1-5, 1.0-10, 1.0-5.0, 2.5-7.5, or 4.0-6.0 ml, optionally 1.0, 2.0, 2.5 or 5.0 ml, optionally 5.0 ml.
33. The pharmaceutical composition of any one of embodiments 1-32, wherein the adenoviral-based delivery and expression system is a recombinant adenoviral vector or a helper-dependent adenoviral vector, and the concentration of vector is reduced to 10%, 20%, 30%, 40%, 50%, 60%, 70%, 80% or 90%, or a range defined by any two of the preceding values, optionally 10%-90%, 10%-50%, 30%-80%, 50%-90%, of a typical and/or publicly disclosed concentration of the recombinant adenoviral vector or helper-dependent adenoviral vector for administration by the same route of administration to the same species of animal for treatment of the same or similar disease or disorder.
34. The pharmaceutical composition of any one of embodiments 1-33, wherein the concentration of the corticosteroid is sufficient to improve one or more of transduction, expression, efficacy and/or safety of the adenoviral-based delivery and expression system when administered to an animal as compared to administration of the same pharmaceutical composition without the corticosteroid.
35. The pharmaceutical composition of embodiment 34, wherein the improvement comprises an improvement in the quantity or duration of a measure of transduction or expression.
36. The pharmaceutical composition of embodiment 34 or 35, wherein the improvement in transfection and/or expression is measured in vitro.
37. The pharmaceutical composition of any one of embodiments 34-36, wherein the improvement in safety comprises a reduction in the number, severity or duration of an adverse event, optionally wherein the adverse event is selected from pain, swelling, effusion, inflammation, headache, fever, chills, and flu-like symptoms.
38. The pharmaceutical composition of any one of embodiments 34-37, wherein the improvement in efficacy comprises an improvement in the onset, amount and/or duration of a therapeutic effect of the adenoviral-based delivery and expression system.
39. The pharmaceutical composition of embodiment 38, wherein the therapeutic effect comprises a reduction in a measure of a symptom or marker of a disease or disorder; optionally wherein the symptom is pain in a joint of the animal, optionally as measured using the Western Ontario and McMaster Universities Osteoarthritis Index pain score (WOMAC-A).
40. The pharmaceutical composition of any one of embodiment 34-39, wherein the improvement is, or is at least, 10%, 20%, 30%, 40%, 50%, 60%, 70%, 80%, 90%, 100%, 125%, 150%, 175%, 200%, 300%, 400%, 500%, 1000%, or 5000%, or a range defined by any two of the preceding values, optionally 10%-5000%, 10%-100%, 100%-5000%, or 50%-500%, as compared to the same pharmaceutical composition without the corticosteroid.
41. The pharmaceutical composition of any one of embodiments 1-40, wherein the concentration of the corticosteroid is, is at least, is not more than, 0.001, 0.01, 0.1, 1.0, 5.0, 10, 15, 20, 25, 30, 40, 50, 60, 70, 80, 90, 100, 250, 500, 750, 1000, 2500, or 5000, or a range defined by any two of the preceding values, optionally 0.001-5000, 0.001-0.1, 0.001-10, 0.1-100, 1.0-100, 1.0-50, 30-300, 25-60, 30-50, 100-1000, 500-5000 mg/ml, optionally 40 mg/ml, of the pharmaceutical composition.
42. The pharmaceutical composition of any one of embodiments 1-41, wherein the pharmaceutical composition is a liquid having a volume that is, is at least, or is not more than, 0.1, 0.25, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10 ml, or a range defined by any two of the preceding values, optionally 0.1-10, 0.1-5, 1.0-10, 1.0-5.0, 2.5-7.5, or 4.0-6.0 ml, optionally 1.0, 2.0, 2.5 or 5.0 ml, optionally 1.0 ml.
43. The pharmaceutical composition of any one of embodiments 1-42, wherein the amount of the adenoviral-based biological delivery and expression system is a therapeutically effective amount.
44. The pharmaceutical composition of any one of embodiments 1-43, wherein the corticosteroid is in a separate dosage form from the adenoviral-based biological delivery and expression system such that the corticosteroid can be administered before or after the adenoviral-based biological delivery and expression system.
45. The pharmaceutical composition of any one of embodiments 1-42, wherein the corticosteroid is in the same dosage form as the adenoviral-based biological delivery and expression system such that the corticosteroid is administered simultaneously with the adenoviral-based biological delivery and expression system, optionally wherein the dosage form is a single fluid.
46. The pharmaceutical composition of embodiment 45, wherein the dosage form comprising the corticosteroid and the adenoviral-based biological delivery and expression system is prepared by combining a composition comprising the corticosteroid and a composition comprising the adenoviral-based biological delivery and expression system.
47. A kit comprising the pharmaceutical composition of embodiment 44, wherein the kit comprises a first container comprising the corticosteroid in a first dosage form, and a second container comprising the adenoviral-based biological delivery and expression system in a second dosage form.
48. A kit for use in preparing the pharmaceutical composition of embodiment 46, wherein the kit comprises the composition comprising the corticosteroid in a first container and the composition comprising the adenoviral-based biological delivery and expression system in a second container.
49. A method comprising administering to an animal the pharmaceutical composition of any one embodiments 1-46.
50. The method of embodiment 49, wherein the administration of the pharmaceutical composition infects cells of the animal with the adenoviral-based delivery and expression system, resulting in expression of the protein encoded by the nucleic acid of adenoviral-based delivery and expression system.
51. The method of embodiment 49, wherein the administration of the pharmaceutical composition infects joint cells of an osteoarthritis-affected joint of a human in need thereof, optionally wherein the joint is a knee joint.
52. The method of any one of embodiment 49-51, wherein the concentration of the corticosteroid is sufficient to improve one or more of transduction, expression, efficacy and/or safety of the adenoviral-based delivery and expression system when administered to the animal as compared to administration of the same pharmaceutical composition without the corticosteroid.
53. The method of embodiment 52, wherein the improvement comprises an improvement in the quantity or duration of a measure of transduction or expression.
54. The method of embodiment 52 or 53, wherein the improvement in transfection and/or expression is measured in vitro.
55. The method of any one of embodiments 52-54, wherein the improvement in safety comprises a reduction in the number, severity or duration of an adverse event, optionally wherein the adverse event is selected from pain, swelling, effusion, inflammation, headache, fever, chills, and flu-like symptoms.
56. The method of any one of embodiments 49-55, wherein the improvement in efficacy comprises an improvement in the onset, amount or duration of a therapeutic effect of the adenoviral-based delivery and expression system.
57. The method of embodiment 56, wherein the therapeutic effect comprises a reduction in a measure of a symptom or marker of a disease or disorder; optionally wherein the symptom is pain in a joint of the animal, optionally as measured using the Western Ontario and McMaster Universities Osteoarthritis Index pain score (WOMAC-A).
58. The method of any one of embodiments 56-57, wherein the improvement in efficacy comprises an improvement in the onset and/or amount of reduction in a measure of joint pain in the animal.
59. The method of any one of embodiments 56-58, wherein the improvement in efficacy comprises an improvement in the likelihood that the animal will experience at least a 50% reduction in a measure of joint pain.
60. The method of any one of embodiments 49-59, wherein the improvement is, or is at least, 10%, 20%, 30%, 40%, 50%, 60%, 70%, 80%, 90%, 100%, 125%, 150%, 175%, 200%, 300%, 400%, 500%, 1000%, or 5000%, or a range defined by any two of the preceding values, optionally 10%-5000%, 10%-100%, 100%-5000%, or 50%-500%, as compared to administration of the same pharmaceutical composition without the corticosteroid.
61. The method of any one of embodiments 49-60, wherein the corticosteroid is administered to the animal a period of time that is, or is not more than 0.00, 0.01, 0.05, 0.1, 0.2, 0.3, 0.4, 0.5, 1, 1.5, 2, 2.5, 3, 4, or 5 hours, or a range defined by any two of the preceding values, optionally 0.0-5, 0.0-3, 0.0-2, 0.0-1, 0.0-0.5, 0.0-0.2, or 0.0-0.01 hours prior to the administration of the adenoviral-based delivery and expression system.
62. The method of any one of embodiments 49-61, wherein the corticosteroid is administered to the animal a period of time that is, or is not more than 0.00, 0.01, 0.05, 0.1, 0.2, 0.3, 0.4, 0.5, 1, 1.5, 2, 2.5, 3, 4, or 5 hours, or a range defined by any two of the preceding values, optionally 0.0-5, 0.0-3, 0.0-2, 0.0-1, 0.0-0.5, 0.0-0.2, or 0.0-0.01 hours after the administration of the adenoviral-based delivery and expression system.
63. The method of any one of embodiments 49-62, wherein the corticosteroid is administered to the animal not more than 3 hours before and not more than 30 minutes after the administration of the adenoviral-based delivery and expression system, wherein the corticosteroid is administered to the animal not more than 30 minutes before and not more than 30 minutes after the administration of the adenoviral-based delivery and expression system, or wherein the corticosteroid is administered to the animal not more than 5 minutes before and not more than 5 minutes after the administration of the adenoviral-based delivery and expression system.
64. The method of any one of embodiments 49-63, wherein the corticosteroid is administered to the animal simultaneously with the adenoviral-based delivery and expression system.
65. The method of embodiment 64, wherein the corticosteroid and the adenoviral-based delivery and expression system are in a single dosage form.
66. The method of any one of embodiments 49-65, wherein the animal has not been administered a corticosteroid, other than the corticosteroid of the pharmaceutical composition, for a period of time that is, or is at least, 2, 3, 4, 5, 6, 12, 18, 24, 48, or 72 hours, or a range defined by any two of the preceding values, optionally 2-72, 3-72, 4-72, 5-72, 2-24, 3-24, 5-24, 2-48, 3-48, 4-48, 5-48, 6-72, 6-48, 12-72, or 18-72 hours prior to administration of the adenoviral-based delivery and expression system.
67. The method of any one of embodiments 49-66, wherein the method comprises administration of the corticosteroid of the pharmaceutical composition systemically and administration of the adenoviral-based delivery and expression system of the pharmaceutical composition locally.
68. The method of any one of embodiments 49-66, wherein the method comprises administration of the corticosteroid and adenoviral-based delivery and expression system of the pharmaceutical composition locally to the same location of the animal.
69. The method of embodiment 68, wherein the method comprises administration of the corticosteroid and adenoviral-based delivery and expression system of the pharmaceutical composition topically, by injection, by inhalation, intra-ocularly, or intra-ear.
70. The method of embodiment 68, wherein the method comprises administration of the corticosteroid and adenoviral-based delivery and expression system of the pharmaceutical composition by injection, wherein the administration of the corticosteroid and the adenoviral-based delivery and expression system is through the same needle and the needle is maintained in the same site during administration of both the corticosteroid and the adenoviral-based delivery and expression system, optionally wherein the needle is flushed with a fluid between administration of the corticosteroid and the adenoviral-based delivery and expression system.
71. The method of any one of embodiments 68-70, wherein the method comprises injection into a joint of the animal, optionally intra-tendinous, intra-muscular, intra-articular, or sub-acromial injection.
72. The method of embodiment 71, wherein the injection is an intra-articular injection, optionally wherein the joint is the knee.
73. The method of any one of embodiments 68-70, wherein the method comprises injection into an intervertebral disc; optionally wherein the injection is intradiscal injection, optionally into the nucleus pulposus (NP) region of the intervertebral disc.
74. The method of any one of embodiments 49-72, wherein the method does not comprise administration into an intervertebral disc, wherein the method does not comprise administration by intradiscal injection, or wherein the method does not comprise intradiscal injection into the nucleus pulposus (NP) or central gelatinous nucleus pulposus (NP) region of the intervertebral disc.
75. The method of any one of embodiments 49-74, wherein the method comprises treating a disease or condition.
76. The method of embodiment 75, wherein the method comprises identifying the animal as suffering from or at risk of suffering from the treated disease or condition prior to administration of the pharmaceutical composition.
77. The method of embodiment 75 or 76, wherein the disease or condition is:
   camptodactyly-arthropathy-coxa vara-pericarditis (CACP) syndrome;
   a musculoskeletal disease or condition;
   a joint disease or condition;
   a lung disease or condition;
   an eye disease or condition;
   an ear disease or condition;
   arthropathies, arthritis, or arthritis-related conditions;
   osteoarthritis, rheumatoid arthritis, gout and pseudo-gout, septic arthritis, psoriatic arthritis, ankylosing spondylitis, juvenile idiopathic arthritis, Still's disease, Reiter's syndrome, or tendinopathies including tendonitis, tendinosis, and tenosynovitis;
   synovial disorders including synovitis;
   a bursa disorder, including bursitis; or
   equine musculoskeletal disorders including bone spavin, navicular syndrome, and osselet.
78. The method of embodiment 75 or 76, wherein the disease or condition is degenerative disc disease (DDD) or a condition associated with DDD.
79. The method of embodiment 75 or 76, wherein the disease or condition is not degenerative disc disease (DDD) or a condition associated with DDD.
80. The method of embodiment 75 or 76, wherein the disease or condition is osteoarthritis or an osteoarthritic condition, optionally of the knee.
81. The method of any one of embodiments 49-80, wherein the animal is a mammal.
82. The method of any one of embodiments 49-80, wherein the animal is a human.
83. The method of any one of embodiments 49-80, wherein the animal is a horse, a dog or a cat.
84 The method of any one of embodiments 49-83, wherein the amount of vector administered to the animal is reduced to 10%, 20%, 30%, 40%, 50%, 60%, 70%, 80% or 90%, or a range defined by any two of the preceding values, optionally 10%-90%, 10%-50%, 30%-80%, 50%-90%, of a typical and/or publicly disclosed amount of the recombinant adenoviral vector or a helper-dependent adenoviral vector administered by the same route of administration to the same species of animal for treatment of the same or similar disease or disorder.
85. The method of any one of embodiments 49-84, wherein the adenoviral-based delivery and expression system is a recombinant adenoviral vector or a helper-dependent adenoviral vector, and the concentration of vector is, is at least, or is not more than, 1.4×10⁵, 1.4×10⁶, 2.8×10⁶, 7.0×10⁶, 1.4×10⁷, 2.8×10⁷, 7.0×10⁷, 1.4×10⁸, 2.8×10⁸, 7.0×10⁸, 1.4×10⁹, 2.8×10⁹, 7.0×10⁹, 1.4×10¹⁰, 2.8×10¹⁰, 7.0×10¹⁰, 1.4×10¹¹, 2.8×10¹¹, 7.0×10¹¹, 1.4×10¹², 2.8×10¹², 7.0×10¹², 1.4×10¹³, 2.8×10¹³, 7.0×10¹³ or a range defined by any two of the preceding values, optionally 1.4×10⁵ to 7.0×10¹³, 1.4×10⁵ to 7.0×10¹², 1.4×10⁵ to 7.0×10¹¹, 1.4×10⁵ to 7.0×10¹⁰, 1.4×10⁵ to 7.0×10⁹, 1.4×10⁵ to 7.0×10⁸, 1.4×10⁹ to 7.0×10¹⁰, optionally 2.8×10⁹, 2.8×10¹⁰, 2.8×10¹¹, genome copies (GC) of recombinant adenoviral vector or a helper-dependent adenoviral vector per ml of the pharmaceutical composition.
86. The method of any one of embodiments 49-84, wherein the adenoviral-based delivery and expression system is a recombinant adenoviral vector or a helper-dependent adenoviral vector, and the concentration of vector is, is at least, or is not more than, 1.4×10⁵, 1.4×10⁶, 2.8×10⁶, 7.0×10⁶, 1.4×10⁷, 2.8×10⁷, 7.0×10⁷, 1.4×10⁸, 2.8×10⁸, 7.0×10⁸, 1.4×10⁹, 2.8×10⁹, 7.0×10⁹, 1.4×10¹⁰, 2.8×10¹⁰, 7.0×10¹⁰, 1.4×10¹¹, 2.8×10¹¹, 7.0×10¹¹, 1.4×10¹², 2.8×10¹², 7.0×10¹², 1.4×10¹³, 2.8×10¹³, 7.0×10¹³ or a range defined by any two of the preceding values, optionally 1.4×10⁵ to 7.0×10¹³, 1.4×10⁵ to 7.0×10¹², 1.4×10⁵ to 7.0×10¹¹, 1.4×10⁵ to 7.0×10¹⁰, 1.4×10⁵ to 7.0×10⁹, 1.4×10⁵ to 7.0×10⁸, 1.4×10⁹ to 7.0×10¹⁰, viral particles (VP) of recombinant adenoviral vector or a helper-dependent adenoviral vector per ml of the pharmaceutical composition.
87. The method of any one of embodiments 49-86, wherein the pharmaceutical composition is a liquid having a volume that is, is at least, or is not more than, 0.1, 0.25, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10 ml, or a range defined by any two of the preceding values, optionally 0.1-10, 0.1-5, 1.0-10, 1.0-5.0, 2.5-7.5, or 4.0-6.0 ml, optionally 2.0, 2.5 or 5.0 ml, optionally 5.0 ml.
88. The method of any one of embodiments 49-87, wherein the pharmaceutical composition is a liquid, and wherein the amount pharmaceutical composition administered to the animal is, is at least, or is not more than, 0.1, 0.25, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10 ml, or a range defined by any two of the preceding values, optionally 0.1-10, 0.1-5, 1.0-10, 1.0-5.0, 2.5-7.5, or 4.0-6.0 ml, optionally 1.0, 2.0, 2.5 or 5.0 ml, optionally 5.0 ml.
89. The method of any one of embodiments 49-88, wherein the adenoviral-based delivery and expression system is a recombinant adenoviral vector or a helper-dependent adenoviral vector, and the amount of vector administered to the animal is, is at least, or is not more than, 1.4×10⁵, 1.4×10⁶, 2.8×10⁶, 7.0×10⁶, 1.4×10⁷, 2.8×10⁷, 7.0×10⁷, 1.4×10⁸, 2.8×10⁸, 7.0×10⁸, 1.4×10⁹, 2.8×10⁹, 7.0×10⁹, 1.4×10¹⁰, 2.8×10¹⁰, 7.0×10¹⁰, 1.4×10¹¹, 2.8×10¹¹, 7.0×10¹¹, 1.4×10¹², 2.8×10¹², 7.0×10¹², 1.4×10¹³, 2.8×10¹³, 7.0×10¹³ or a range defined by any two of the preceding values, optionally 1.4×10⁵ to 7.0×10¹³, 1.4×10⁵ to 7.0×10¹², 1.4×10⁵ to 7.0×10¹¹, 1.4×10⁵ to 7.0×10¹⁰, 1.4×10⁵ to 7.0×10⁹, 1.4×10⁵ to 7.0×10⁸, 1.4×10⁹ to 7.0×10¹⁰, optionally 1.4×10⁵, 1.4×10⁷, 5.6×10⁹, 7.0×10⁹, 1.4×10¹⁰, 1.4×10¹¹, or 1.4×10¹², optionally 5.6×10⁹, 7.0×10⁹, 1.4×10¹⁰, 1.4×10¹¹, or 1.4×10¹², genome copies (GC) of recombinant adenoviral vector or a helper-dependent adenoviral vector.
90. The method of any one of embodiments 49-88, wherein the adenoviral-based delivery and expression system is a recombinant adenoviral vector or a helper-dependent adenoviral vector, and the amount of vector administered to the animal is, is at least, or is not more than, 1.4×10⁵, 1.4×10⁶, 2.8×10⁶, 7.0×10⁶, 1.4×10⁷, 2.8×10⁷, 7.0×10⁷, 1.4×10⁸, 2.8×10⁸, 7.0×10⁸, 1.4×10⁹, 2.8×10⁹, 7.0×10⁹, 1.4×10¹⁰, 2.8×10¹⁰, 7.0×10¹⁰, 1.4×10¹¹, 2.8×10¹¹, 7.0×10¹¹, 1.4×10¹², 2.8×10¹², 7.0×10¹², 1.4×10¹³, 2.8×10¹³, 7.0×10¹³ or a range defined by any two of the preceding values, optionally 1.4×10⁵ to 7.0×10¹³, 1.4×10⁵ to 7.0×10¹², 1.4×10⁵ to 7.0×10¹¹, 1.4×10⁵ to 7.0×10¹⁰, 1.4×10⁵ to 7.0×10⁹, 1.4×10⁵ to 7.0×10⁸, 1.4×10⁹ to 7.0×10¹⁰, optionally 1.4×10⁵, 1.4×10⁷, 5.6×10⁹, 7.0×10⁹, 1.4×10¹⁰, 1.4×10¹¹, or 1.4×10¹², optionally 5.6×10⁹, 7.0×10⁹, 1.4×10¹⁰, 1.4×10¹¹, or 1.4×10¹², viral particles (VP) of recombinant adenoviral vector or a helper-dependent adenoviral vector.
91. The method of any one of embodiments 49-90, wherein the concentration of the corticosteroid is, is at least, is not more than, 0.001, 0.01, 0.1, 1.0, 5.0, 10, 15, 20, 25, 30, 40, 50, 60, 70, 80, 90, 100, 250, 500, 750, 1000, 2500, or 5000, or a range defined by any two of the preceding values, optionally 0.001-5000, 0.001-0.1, 0.001-10, 0.1-100, 1.0-100, 1.0-50, 30-300, 25-60, 30-50, 100-1000, 500-5000 mg/ml, optionally 40 mg/ml, of the pharmaceutical composition. 92. The method of any one of embodiments 49-91, wherein the pharmaceutical composition is a liquid having a volume that is, is at least, or is not more than, 0.1, 0.25, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10 ml, or a range defined by any two of the preceding values, optionally 0.1-10, 0.1-5, 1.0-10, 1.0-5.0, 2.5-7.5, or 4.0-6.0 ml, optionally 1.0, 2.0, 2.5 or 5.0 ml, optionally 1.0 ml.
93. The method of any one of embodiments 49-92, wherein the pharmaceutical composition is a liquid and wherein the amount pharmaceutical composition administered to the animal is, is at least, or is not more than, 0.1, 0.25, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10 ml, or a range defined by any two of the preceding values, optionally 0.1-10, 0.1-5, 1.0-10, 1.0-5.0, 2.5-7.5, or 4.0-6.0 ml, optionally 1.0, 2.0, 2.5 or 5.0 ml, optionally 1.0 ml.
94. The method of any one of embodiments 49-93, wherein the amount of corticosteroid administered to the animal is, is at least, is not more than, 0.001, 0.01, 0.1, 1.0, 5.0, 10, 15, 20, 25, 30, 40, 50, 60, 70, 80, 90, 100, 250, 500, 750, 1000, 2500, or 5000, or a range defined by any two of the preceding values, optionally 0.001-5000, 0.001-0.1, 0.001-10, 0.1-100, 1.0-100, 1.0-50, 30-300, 25-60, 30-50, 100-1000, 500-5000 mg, optionally 40 mg.
95. The method of any one of embodiments 49-94, wherein the method comprises:
   identifying a human subject as suffering from osteoarthritis of the knee,
   administering the pharmaceutical composition to the human subject by intra-articular injection into an osteoarthritis-affected knee joint,
   wherein the corticosteroid is in a separate dosage form from the adenoviral-based biological delivery and expression system,
   wherein administration of the corticosteroid comprises intra-articular injection of 1 ml of a dosage form comprising 40 mg/ml of the corticosteroid,
   wherein administration of the adenoviral-based biological delivery and expression system comprises intra-articular injection of 2, 2.5 or 5 ml of a dosage form comprising optionally 2.8×10⁹, 2.8×10¹⁰, 2.8×10¹¹ per ml of the adenoviral-based biological delivery and expression system,
   wherein the amount of adenoviral-based biological delivery and expression system delivered to the knee is 5.6×10⁹, 7.0×10⁹, 1.4×10¹⁰, 1.4×10¹¹, or 1.4×10¹², genome copies (GC) of recombinant adenoviral vector or a helper-dependent adenoviral vector,
   wherein the administration of the corticosteroid and the adenoviral-based delivery and expression system is through the same needle and the needle is maintained in the same site during administration of both the corticosteroid and the adenoviral-based delivery and expression system, optionally wherein the needle is flushed with a fluid between corticosteroid and the adenoviral-based delivery and expression system, and
   wherein the corticosteroid is administered not more than 0-15 minutes, optionally 0-5 minutes, before administration of the adenoviral-based delivery and expression system.
96. The method of embodiment 95, wherein the adenoviral-based delivery and expression system comprises a helper-dependent adenoviral vector comprising a nucleic acid sequence encoding human IL-1Ra protein, left and right inverted terminal repeats, an adenoviral packaging signal and non-viral, and non-coding stuffer nucleic acid sequences, wherein the expression of the nucleic acid sequence encoding human IL-1Ra protein is regulated by a NF-κB inducible promoter, which is located upstream of the reading frame of the nucleic acid sequence encoding the human IL-1Ra protein.
97. The method of embodiment 96, wherein the genome of helper-dependent adenoviral vector has nucleic acid sequence of SEQ ID NO: 7.
98. The method of any one of embodiments 95-97, wherein the corticosteroid is methylprednisolone.
99. The method of any one of embodiments 49-98, wherein the amount of the adenoviral-based biological delivery and expression system administered to the animal is a therapeutically effective amount.
100. Use of the composition or kit of any one of embodiments 1-48 for use in a method of treating a disease or condition, the method comprising the method of any one of embodiments 49-98.
101. A kit comprising a first container comprising a corticosteroid in a first dosage form, and a second container comprising an adenoviral-based biological delivery and expression system in a second dosage form.
102. The kit according to embodiment 101, wherein the corticosteroid or the adenoviral-based biological delivery and expression system is provided as recited in any one of embodiments 1-46.
103. A pharmaceutical composition comprising a corticosteroid and an adenoviral-based delivery and expression system according to any one of embodiments 1-46, or a kit comprising a first container comprising a corticosteroid in a first dosage form, and a second container comprising an adenoviral-based biological delivery and expression system in a second dosage form according to any one of embodiments 101-102 for use as a medicament.
104. A pharmaceutical composition comprising a corticosteroid and an adenoviral-based delivery and expression system according to any one of embodiments 1-46, or a kit comprising a first container comprising a corticosteroid in a first dosage form, and a second container comprising an adenoviral-based biological delivery and expression system in a second dosage form according to any one of embodiments 101-102 for use in the prevention or treatment of a disease or condition.
105. The pharmaceutical composition or kit for the use as recited in embodiment 104, wherein the disease of condition is selected from the group consisting of:
   camptodactyly-arthropathy-coxa vara-pericarditis (CACP) syndrome;
   a musculoskeletal disease or condition;
   a joint disease or condition;
   a lung disease or condition;
   an eye disease or condition;
   an ear disease or condition;
   arthropathies, arthritis, and arthritis-related conditions;
   osteoarthritis, rheumatoid arthritis, gout and pseudo-gout, septic arthritis, psoriatic arthritis, ankylosing spondylitis, juvenile idiopathic arthritis, Still's disease, Reiter's syndrome, or tendinopathies including tendonitis, tendinosis, and tenosynovitis;
   synovial disorders including synovitis;
   a bursa disorder, including bursitis; or
   equine musculoskeletal disorders including bone spavin, navicular syndrome, and osselet.
106. The pharmaceutical composition or kit for the use as recited in embodiment 104, wherein the disease or condition is degenerative disc disease (DDD) or a condition associated with DDD.
107. The pharmaceutical composition or kit for the use as recited in embodiment 104, wherein the disease or condition is not degenerative disc disease (DDD) or a condition associated with DDD.
108. The pharmaceutical composition, kit, method or use according to any one of the preceding embodiments wherein the adenoviral-based biological delivery and expression system is PCRX201, optionally wherein the amount of the corticosteroid and/or the PCRX is a therapeutically effective amount.

Any of the above numbered embodiments can be combined with any other numbered embodiment, even where not expressly stated, unless expressly stated otherwise or unless the embodiments are mutually exclusive.

### BRIEF DESCRIPTION OF THE DRAWINGS

FIG. 1 depicts and embodiment of the genome map of Humantakinogene Hadenovec (FX201, also referred to herein as PCRX-201 or PCRX201). ITR=inverted terminal repeats (1-103 bp on 5′; 29,158-29,260 bp on 3′), Y′=packaging signals (240-375 bp), HPRT Stuffer=human hypoxanthine phosphoribosyltransferase (463-16,518 bp), Human Cosmid Insert=human cosmid (16,532-27,637 bp), SV40 Poly A=Simian virus 40 Poly A (27,750-28,020 bp), huIL-1Ra=human interleukin-1 receptor antagonist, the genome of interest (28,033-28,566 bp), NF-κB5-ELAM Promoter=NΦ-κB ινδυχιβλε promoter (28,581-28,842 bp).

FIG. 2 depicts an embodiment of the basic gene map of the helper-dependent adenoviral vector of the disclosure. The vector backbone (excluding the cDNA of the transgene and promoter) consists of the left and right inverted terminal repeats (ITR), adenoviral packaging signal (Ψ) and non-coding, non-viral stuffer sequences (remaining unmarked sequence between ITRs). The cDNA of the protein to be expressed (transgene), for example equine IL-1Ra (GQ-201), murine IL-1Ra, human IL-1Ra, (alternately equine PRG4, canine PRG4, feline PRG4, murine PRG4, not shown) is cloned between the viral left and right ITRs of the used adenoviral vector. The expression of IL-1-Ra is typically controlled by inflammation-sensitive NF-κB5-ELAM promoter. The expression of PRG4 is typically controlled by an EF1 alpha inducible promoter (not shown).

FIG. 3 depicts an embodiment of photomicrographs of X-Gal staining in 116 cells following treatment with HDAd-LacZ at 100 virus particles/cell in combination with the corticosteroid triamcinolone acetonide (TAC) at 0.36 μg/ml or 428.5 μg/ml, or without TAC.

FIG. 4 depicts an embodiment of a preliminary semi-quantitative measure of the X-Gal staining in FIG. 3.

FIG. 5 depicts an embodiment of a photograph of X-Gal staining in HEK293 cells following treatment with HDAd-LacZ at 100, 30, or 10 virus particles/cell in combination with TAC at 0.36 μg/ml or 428.5 μg/ml, or without TAC.

FIG. 6 depicts an embodiment of photomicrographs of the wells in FIG. 5.

FIG. 7 depicts an embodiment of a preliminary semi-quantitative measure of the X-Gal staining in FIG. 5.

FIG. 8 depicts an embodiment of a preliminary semi-quantitative measure of the X-Gal staining in FIG. 6.

FIG. 9 depicts an embodiment of photomicrographs of X-Gal staining in HEK293 following treatment with HDAd-LacZ and TAC (columns A-K: 2.88 μg/mL-0.0028125 μg/mL; column L: 0 ng/ml).

FIG. 10 depicts an embodiment of a preliminary semi-quantitative measure of the X-Gal staining in FIG. 9.

FIG. 11 depicts an embodiment of photomicrographs of X-Gal staining in HEK293 cells following treatment with HDAd-LacZ and TAC (columns B-K: 2.8125 ng/mL-0.005493164 ng/ml; column L: 0 ng/mL).

FIG. 12 depicts an embodiment of a preliminary semi-quantitative measure of the X-Gal staining in FIG. 11.

FIG. 13 depicts an embodiment of a photograph of X-Gal staining in HEK293 cells following treatment with HDAd-LacZ and TAC (rows A-F: 360 ng/ml-0.35 ng/ml; row G: 0 ng/ml; row H: 360 ng/mL) using different timing of the TAC relative to HDAd-LacZ treatment. “PRE” signifies TAC treatment 3 hours prior to HDAd-LacZ infection, “SIMULTANEOUS” signifies TAC treatment and HDAd-LacZ infection simultaneously, and “POST” signifies TAC treatment 3 hours post HDAd-LacZ infection.

FIG. 14 depicts an embodiment of a preliminary semi-quantitative measure of the X-Gal staining in FIG. 13.

FIG. 15 depicts an embodiment of photomicrographs of X-Gal wells in FIG. 13, (rows A-F: 360 ng/ml-0.35 ng/ml; row G: 0 ng/ml; row H: 360 ng/ml) using different timing of the TAC relative to HDAd-LacZ treatment. “PRE” signifies TAC treatment 3 hours prior to HDAd-LacZ infection, “SIMULTANEOUS” signifies TAC treatment and HDAd-LacZ infection simultaneously, and “POST” signifies TAC treatment 3 hours post HDAd-LacZ infection.

FIG. 16 depicts an embodiment of a preliminary semi-quantitative measure of the X-Gal staining in FIG. 15.

FIG. 17 depicts an embodiment of a photograph of X-Gal staining in HEK293 cells following treatment with HDAd-LacZ+Betamethasone or Dexamethasone at various concentrations (columns A-K: 4000 ng/ml-0.0038 ng/mL Betamethasone or Dexamethasone, L: no betamethasone or dexamethasone (−)).

FIG. 18 depicts an embodiment of photomicrographs of X-Gal wells in FIG. 17, (columns A-K: 4000 ng/ml-0.0038 ng/mL Betamethasone or Dexamethasone, L: no betamethasone or dexamethasone (−)).

FIG. 19 depicts an embodiment of a preliminary semi-quantitative measure of the X-Gal staining in FIG. 18.

FIG. 20 depicts an embodiment of a preliminary semi-quantitative measure of the percentage of total length of the synovium of rat knee joint expressing X-Gal staining following intraarticular co-administration of HDAd-LacZ and TAC. HDAd was administered at four doses: 5×10¹⁰, 5×10⁹, 5×10⁸ and 5×10⁷, with (+TAC) or without 0.06 mg of TAC.

FIG. 21 depicts an embodiment of a preliminary semi-quantitative measure of the percentage of total tissue area of the synovium of rat knee joint expressing X-Gal staining following intraarticular co-administration of HDAd-LacZ and TAC. HDAd was administered at four doses: 5×10¹⁰, 5×10⁹, 5×10⁸ and 5×10⁷, with (+TAC) or without 0.06 mg of TAC.

FIG. 22 depicts an embodiment of a preliminary semi-quantitative measure of synovial/periarticular inflammation in the rat knee joint following intraarticular co-administration of HDAd-LacZ and TAC. HDAd was administered at four doses: 5×10¹⁰, 5×10⁹, 5×10⁸ and 5×10⁷, with (+TAC) or without 0.06 mg of TAC.

FIG. 23 depicts an embodiment of a graphical representation of the mean percent change in WOMAC-A pain index in human subjects treated with a low (1.4E10 genome copies (GC)/knee), mid (1.4E11 GC/knee), or high (1.4E12 GC/knee) dose of PCRX administered by intra-articulate injection into the osteoarthritis-affected knee, either with or without administration of 40 mg of methylprednisolone by intra-articulate injection of 1 ml through the same needle just prior to injection of PCRX201.

FIG. 24 depicts an embodiment of a different graphical representation of the mean percent change in WOMAC-A pain index shown in FIG. 23, with additional time points. “Not Pretreated” signifies no methylprednisolone treatment, and “Pretreated” signifies administration of 40 mg of methylprednisolone by intra-articulate injection through the same needle just prior to injection of PCRX201.

FIG. 25 depicts an embodiment of a graphical representation of the percentage of patients with a ≥50% decline from baseline in WOMAC-A pain index. “Non-methylprednisolone” signifies no methylprednisolone treatment, and “Methylprednisolone” signifies administration of 40 mg of methylprednisolone by intra-articulate injection through the same needle just prior to injection of PCRX201.

### DETAILED DESCRIPTION OF THE DISCLOSURE

The present disclosure provides pharmaceutical compositions, kits, methods and uses thereof, that include a corticosteroid and an adenoviral-based delivery and expression system. In some embodiments, the compositions, kits, methods and uses thereof, improve one or more of transduction, expression, efficacy and/or safety of the adenoviral-based delivery and expression system when administered to an animal as compared to administration of the same pharmaceutical composition without the corticosteroid.

### Pharmaceutical Compositions

In some embodiments, in the pharmaceutical composition of any one of embodiments disclosed herein, the pharmaceutical composition includes a corticosteroid and an adenoviral-based delivery and expression system is disclosed. As used herein, “pharmaceutical composition” has its plain and ordinary meaning as understood by one of skill in the art in view of the present disclosure. The pharmaceutical composition need not be a single dosage form, and in some embodiments the corticosteroid and the adenoviral-based delivery and expression system are in separate dosage forms (e.g., two separate liquids) such that they can be administered separately. In some embodiments, the pharmaceutical composition is a single dosage form (e.g., liquid) that contains both the corticosteroid and the adenoviral-based delivery and expression system.

In some embodiments, in the pharmaceutical composition of any one of embodiments disclosed herein the corticosteroid is a glucocorticoid, a mineralocorticoid, or both, for example, the corticosteroid may have predominantly glucocorticoid or mineralocorticoid activity, or the activity of the corticosteroid may be both as a glucocorticoid or mineralocorticoid. In some embodiments, corticosteroid is a combination of one or more glucocorticoids, one or more a mineralocorticoids, or both a glucocorticoid and a mineralocorticoid. In some embodiments, the corticosteroid is selected from the group consisting of: beclomethasone, betamethasone, budesonide, cortisone, deflazacort, dexamethasone, hydrocortisone, methylprednisolone, prednisolone, prednisone, triamcinolone, fludrocortisone, deoxycorticosterone, aldosterone, and mixtures thereof. In some embodiments, the corticosteroid is selected from the group consisting of: beclomethasone, betamethasone, budesonide, cortisone, deflazacort, dexamethasone, hydrocortisone, methylprednisolone, prednisolone, prednisone, triamcinolone, fludrocortisone, deoxycorticosterone, aldosterone, ciclesonide, fluticasone, flunisolide, mometasone, and mixtures thereof. In some embodiments, the corticosteroid is selected from the group consisting of: beclomethasone, betamethasone, budesonide, cortisone, dexamethasone, hydrocortisone, methylprednisolone, prednisolone, prednisone, triamcinolone, and mixtures thereof. In some embodiments, the corticosteroid is selected from the group consisting of: beclomethasone, betamethasone, budesonide, cortisone, dexamethasone, hydrocortisone, methylprednisolone, prednisolone, triamcinolone, and mixtures thereof. In some embodiments, the corticosteroid is selected from the group consisting of: betamethasone, dexamethasone, triamcinolone, methylprednisolone, and mixtures thereof. In some embodiments, the corticosteroid is selected from the group consisting of: beclomethasone, budesonide, ciclesonide, fluticasone, flunisolide, mometasone, and mixtures thereof. In some embodiments, the corticosteroid is selected from the group consisting of: fludrocortisone, deoxycorticosterone, aldosterone, and mixtures thereof. In some embodiments, the corticosteroid is selected from the group consisting of: betamethasone, dexamethasone, triamcinolone, methylprednisolone, and mixtures thereof. In some embodiments, the corticosteroid is methylprednisolone. In some embodiments, the corticosteroid is methylprednisolone acetate. In some embodiments, in the pharmaceutical composition of any one of embodiments disclosed herein the concentration of the corticosteroid is, is at least, is not more than, 0.001, 0.01, 0.1, 1.0, 5.0, 10, 15, 20, 25, 30, 40, 50, 60, 70, 80, 90, 100, 250, 500, 750, 1000, 2500, or 5000, or a range defined by any two of the preceding values, optionally 0.001-5000, 0.001-0.1, 0.001-10, 0.1-100, 1.0-100, 1.0-50, 30-300, 25-60, 30-50, 100-1000, 500-5000 mg/ml, optionally 40 mg/ml, of the pharmaceutical composition. In some embodiments, the pharmaceutical composition is a liquid having a volume that is, is at least, or is not more than, 0.1, 0.25, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10 ml, or a range defined by any two of the preceding values, optionally 0.1-10, 0.1-5, 1.0-10, 1.0-5.0, 2.5-7.5, or 4.0-6.0 ml, optionally 1.0, 2.0, 2.5 or 5.0 ml, optionally 1.0 ml.

In some embodiments, in the pharmaceutical composition of any one of embodiments disclosed herein the adenoviral-based delivery and expression system includes a nucleic acid encoding a protein for expression by the adenoviral-based delivery and expression system; optionally wherein the protein is a therapeutic protein. In some embodiments, the adenoviral-based delivery and expression system is a recombinant adenoviral vector or a helper-dependent adenoviral vector. In some embodiments, the recombinant adenoviral vector or a helper-dependent adenoviral vector is an Ad5 serotype. In some embodiments, the helper-dependent adenoviral vector includes a genome including or consisting of: the nucleic acid sequence encoding the protein; a promoter regulating expression of the nucleic acid sequence encoding the protein; left and right inverted terminal repeats; an adenoviral packaging signal; and non-viral and non-coding stuffer nucleic acid sequences, optionally wherein the promoter is located upstream of the reading frame of the nucleic acid sequence encoding the protein such that the expression of protein is regulated by the promoter. In some embodiments, the promoter is a ubiquitous, constitutive promoter. In some embodiments, the promoter is selected from the group consisting of: elongation factor 1 alpha (EF1 alpha) promoter, cytomegalovirus (CMV) promoter, beta-actin promoter, simian virus 40 (SV40) early promoter, ubiquitin c promoter, glyceraldehyde 3-phosphate dehydrogenase (GAPDH) promoter, and phosphoglycerate kinase (PGK) promoter. In some embodiments, the promoter is an inflammation-sensitive promoter. In some embodiments, the promoter is selected from the group consisting of: a promoter inducible by NF-κB, interleukin 6 (II-6), interleukin-1 (IL-1), tumor necrosis factor (TNF), cyclooxygenase 2 (COX-2), complement factor 3 (C3), serum amyloid A3 (SAA3), and macrophage inflammatory protein-1a (MIP-1a). In some embodiments, the genome includes or consists of a nucleic acid that is, or is at least, 80%, 85%, 90%, 95%, 96%, 97%, 98%, 99%, or 100% identical to SEQ ID NO: 7 or 8, excluding the sequences of the promoter and the nucleic acid sequence encoding the protein from the comparison between the genome and SEQ ID NO: 7 or 8. In some embodiments, the genome includes or consists of a nucleic acid wherein the nucleic acid sequences of the left and right inverted terminal repeats, the adenoviral packaging signal, and the non-viral and non-coding stuffer portions of the genome is, or is at least, 80%, 85%, 90%, 95%, 96%, 97%, 98%, 99%, or 100% identical to SEQ ID NO: 7 or 8.

In some embodiments, in the pharmaceutical composition of any one of embodiments disclosed herein the nucleic acid encoding the protein encodes for a therapeutic protein. In some embodiments, the nucleic acid encoding the protein encodes for: interleukin-1 receptor antagonist (IL-1Ra), proteoglycan 4 (PRG4), or both IL-1Ra and PRG4, or a biologically active fragment of at least one of the preceding. In some embodiments, the nucleic acid encoding the protein encodes for: IL-1Ra, PRG4, or both IL-1Ra and PRG4. In some embodiments, the nucleic acid encoding the protein encodes for IL-1Ra. In some embodiments, the nucleic acid encoding the protein encodes for PRG4. In some embodiments, the nucleic acid encodes for a mammalian protein. In some embodiments, the nucleic acid encodes for an equine, canine, feline, or murine protein. In some embodiments, the nucleic acid encodes for a human protein. In some embodiments, the nucleic acid encoding the protein is codon optimized. In some embodiments, the nucleic acid encoding the protein includes or consists of a nucleic acid that is, or is at least, 80%, 85%, 90%, 95%, 96%, 97%, 98%, 99%, or 100% identical or homologous to a sequence selected from SEQ ID NOs: 1, 4, 5, 10, 11, 14, 17, 18 or 19, optionally wherein the sequence is codon optimized. In some embodiments, the genome includes or consists of a nucleic acid that is, or is at least, 80%, 85%, 90%, 95%, 96%, 97%, 98%, 99%, or 100% identical to SEQ ID NOs: 2, 3, 7, 8, 9. In some embodiments, the nucleic acid encodes a PRG4 protein including or consisting of an amino acid sequence that is, or is at least, 80%, 85%, 90%, 95%, 96%, 97%, 98%, 99%, or 100% identical or homologous to a sequence selected from SEQ ID NO: 12, 13, 17, 18, or 19, or a biologically active fragment thereof, or a homolog thereof from any other species. In some embodiments, the nucleic acid encodes an IL-1Ra protein including or consisting of an amino acid sequence that is, or is at least, 80%, 85%, 90%, 95%, 96%, 97%, 98%, 99%, or 100% identical or homologous to a sequence selected from SEQ ID NO: 6, 15 or 16, or a biologically active fragment thereof, or a homolog thereof from any other species. In some embodiments, the nucleic acid encodes for PRG4, and expression of PRG4 is regulated by a ubiquitous, constitutive promoter. In some embodiments, the nucleic acid encodes for PRG4, and expression of PRG4 is regulated by a promoter selected from the group consisting of elongation factor 1 alpha (EF1 alpha) promoter, cytomegalovirus (CMV) promoter, beta-actin promoter, simian virus 40 (SV40) early promoter, ubiquitin c promoter, glyceraldehyde 3-phosphate dehydrogenase (GAPDH) promoter, and phosphoglycerate kinase (PGK) promoter. In some embodiments, the nucleic acid encodes for IL-1Ra, and expression of IL-1Ra is regulated by an inflammation-sensitive promoter. In some embodiments, the nucleic acid encodes for IL-1Ra, and expression of IL-1Ra is regulated by an inflammation-sensitive promoter selected from the group consisting of a promoter inducible by NF-κB, interleukin 6 (II-6), interleukin-1 (IL-1), tumor necrosis factor (TNF), cyclooxygenase 2 (COX-2), complement factor 3 (C3), serum amyloid A3 (SAA3), and macrophage inflammatory protein-1a (MIP-1a). In some embodiments, the pharmaceutical composition of any one of embodiments 7-17, wherein the promoter is located upstream of the reading frame of the nucleic acid sequence encoding the protein, such that the expression of protein is regulated by the promoter.

In some embodiments, in the pharmaceutical composition of any one of embodiments disclosed herein the adenoviral-based biological delivery and expression system includes a helper-dependent adenoviral vector including a nucleic acid sequence encoding animal IL-1Ra protein, left and right inverted terminal repeats, an adenoviral packaging signal and non-viral, and non-coding stuffer nucleic acid sequences, wherein the expression of the nucleic acid sequence encoding animal IL-1Ra protein is regulated by a NF-κB inducible promoter, which is located upstream of the reading frame of the nucleic acid sequence encoding the animal IL-1Ra protein. In some embodiments, the animal is equine, canine, feline or murine.

In some embodiments, in the pharmaceutical composition of any one of embodiments disclosed herein the adenoviral-based biological delivery and expression system includes a helper-dependent adenoviral vector including a nucleic acid sequence encoding human IL-1Ra protein, left and right inverted terminal repeats, an adenoviral packaging signal and non-viral, and non-coding stuffer nucleic acid sequences, wherein the expression of the nucleic acid sequence encoding human IL-1Ra protein is regulated by a NF-κB inducible promoter, which is located upstream of the reading frame of the nucleic acid sequence encoding the human IL-1Ra protein.

In some embodiments, in the pharmaceutical composition of any one of embodiments disclosed herein the adenoviral-based biological delivery and expression system includes a helper-dependent adenoviral vector including a nucleic acid sequence encoding animal PRG4 protein, left and right inverted terminal repeats, an adenoviral packaging signal and non-viral, and non-coding stuffer nucleic acid sequences, wherein the expression of the nucleic acid sequence encoding animal PRG4 protein is regulated by a EF1 alpha inducible promoter, which is located upstream of the reading frame of the nucleic acid sequence encoding the animal PRG4 protein. In some embodiments, the animal is equine, canine, feline or murine.

In some embodiments, in the pharmaceutical composition of any one of embodiments disclosed herein the adenoviral-based biological delivery and expression system includes a helper-dependent adenoviral vector including a nucleic acid sequence encoding human PRG4 protein, left and right inverted terminal repeats, an adenoviral packaging signal and non-viral, and non-coding stuffer nucleic acid sequences, wherein the expression of the nucleic acid sequence encoding human PRG4 protein is regulated by a EF1 alpha inducible promoter, which is located upstream of the reading frame of the nucleic acid sequence encoding the human PRG4 protein.

In some embodiments, in the pharmaceutical composition of any one of embodiments disclosed herein the pharmaceutical composition is formulated for localized administration. In some embodiments, the pharmaceutical composition is formulated for localized administration topically, by injection, by inhalation, intra-ocularly, or intra-ear. In some embodiments, the pharmaceutical composition is formulated for injection into a joint. In some embodiments, the pharmaceutical composition is formulated for injection into a joint wherein the injection is intra-tendinous, intra-muscular, intra-articular, or sub-acromial injection. In some embodiments, the pharmaceutical composition is formulated for intra-articular injection into a human joint. In some embodiments, the joint is the knee.

In some embodiments, in the pharmaceutical composition of any one of embodiments disclosed herein the pharmaceutical composition is formulated for injection into an intervertebral disc. In some embodiments, the pharmaceutical composition is formulated for injection into an intervertebral disc wherein the injection is intradiscal injection. In some embodiments, the injection is into the nucleus pulposus (NP) region of the intervertebral disc.

In some embodiments, in the pharmaceutical composition of any one of embodiments disclosed herein the pharmaceutical composition is not formulated for administration into an intervertebral disc. In some embodiments, the pharmaceutical composition is not formulated for administration into an intervertebral disc wherein the administration is intradiscal injection. In some embodiments, the pharmaceutical composition is not formulated for administration into an intervertebral disc, wherein the administration is intradiscal injection into the nucleus pulposus (NP) or central gelatinous nucleus pulposus (NP) region of the intervertebral disc.

In some embodiments, the pharmaceutical composition of the disclosure is not a pharmaceutical composition for injecting directly to the intervertebral disc that further includes a corticosteroid, such as methylprednisolone, betamethasone, or triamcinolone, plus a small amount of a local anesthetic, such as lidocaine or bupivacaine. In some embodiments, the pharmaceutical composition of the disclosure is not a pharmaceutical composition for injecting directly to the intervertebral disc that further includes a corticosteroid. In some embodiments, the pharmaceutical composition of the disclosure is not a pharmaceutical composition for injecting directly to the intervertebral disc that further includes a corticosteroid, wherein the corticosteroid is methylprednisolone, betamethasone, triamcinolone, or combinations thereof. In some embodiments, the pharmaceutical composition of the disclosure is not a pharmaceutical composition for injecting directly to the intervertebral disc, wherein the corticosteroid, for example, methylprednisolone, betamethasone, triamcinolone, or combinations thereof, is in a separate dosage form from the adenoviral-based biological delivery and expression system, such that the corticosteroid can be administered before or after the adenoviral-based biological delivery and expression system. In some embodiments, the pharmaceutical composition of the disclosure is not a pharmaceutical composition for injecting directly to the intervertebral disc, wherein the corticosteroid, for example, methylprednisolone, betamethasone, triamcinolone, or combinations thereof, is in the same dosage form (e.g., a single fluid) as the adenoviral-based biological delivery and expression system, such that the corticosteroid is administered simultaneously with the adenoviral-based biological delivery and expression system.

In some embodiments, in the pharmaceutical composition of any one of embodiments disclosed herein the pharmaceutical composition is a liquid. In some embodiments, in the pharmaceutical composition of any one of embodiments disclosed herein, wherein the adenoviral-based delivery and expression system is in a buffer including TRIS 10 mM, NaCl 75 mM, Polysorbate 80 0.02% (v/v), sucrose 5% (w/v), MgCl₂ 1.0 mM, EDTA 100 μM, ethanol 0.5% (v/v), and L-histidine 10 mM. In some embodiments, the adenoviral-based delivery and expression system is a recombinant adenoviral vector or a helper-dependent adenoviral vector, and the concentration of vector is, is at least, or is not more than, 1.4×10⁵, 1.4×10⁶, 2.8×10⁶, 7.0×10⁶, 1.4×10⁷, 2.8×10⁷, 7.0×10⁷, 1.4×10⁸, 2.8×10⁸, 7.0×10⁸, 1.4×10⁹, 2.8×10⁹, 7.0×10⁹, 1.4×10¹⁰, 2.8×10¹⁰, 7.0×10¹⁰, 1.4×10¹¹, 2.8×10¹¹, 7.0×10¹¹, 1.4×10¹², 2.8×10¹², 7.0×10¹², 1.4×10¹³, 2.8×10¹³, 7.0×10¹³ or a range defined by any two of the preceding values, optionally 1.4×10⁵ to 7.0×10¹³, 1.4×10⁵ to 7.0×10¹², 1.4×10⁵ to 7.0×10¹¹, 1.4×10⁵ to 7.0×10¹⁰, 1.4×10⁵ to 7.0×10⁹, 1.4×10⁵ to 7.0×10⁸, 1.4×10⁹ to 7.0×10¹⁰, optionally 2.8×10⁹, 2.8×10¹⁰, 2.8×10¹¹, genome copies (GC) of recombinant adenoviral vector or a helper-dependent adenoviral vector per ml of the pharmaceutical composition. In some embodiments, the adenoviral-based delivery and expression system is a recombinant adenoviral vector or a helper-dependent adenoviral vector, and the concentration of vector is, is at least, or is not more than, 1.4×10⁵, 1.4×10⁶, 2.8×10⁶, 7.0×10⁶, 1.4×10⁷, 2.8×10⁷, 7.0×10⁷, 1.4×10⁸, 2.8×10⁸, 7.0×10⁸, 1.4×10⁹, 2.8×10⁹, 7.0×10⁹, 1.4×10¹⁰, 2.8×10¹⁰, 7.0×10¹⁰, 1.4×10¹¹, 2.8×10¹¹, 7.0×10¹¹, 1.4×10¹², 2.8×10¹², 7.0×10¹², 1.4×10¹³, 2.8×10¹³, 7.0×10¹³ or a range defined by any two of the preceding values, optionally 1.4×10⁵ to 7.0×10¹³, 1.4×10⁵ to 7.0×10¹², 1.4×10⁵ to 7.0×10¹¹, 1.4×10⁵ to 7.0×10¹⁰, 1.4×10⁵ to 7.0×10⁹, 1.4×10⁵ to 7.0×10⁸, 1.4×10⁹ to 7.0×10¹⁰, viral particles (VP) of recombinant adenoviral vector or a helper-dependent adenoviral vector per ml of the pharmaceutical composition. In some embodiments, the pharmaceutical composition is a liquid having a volume that is, is at least, or is not more than, 0.1, 0.25, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10 ml, or a range defined by any two of the preceding values, optionally 0.1-10, 0.1-5, 1.0-10, 1.0-5.0, 2.5-7.5, or 4.0-6.0 ml, optionally 1.0, 2.0, 2.5 or 5.0 ml, optionally 5.0 ml. In some embodiments, the adenoviral-based delivery and expression system is a recombinant adenoviral vector or a helper-dependent adenoviral vector, and the concentration of vector is reduced to 10%, 20%, 30%, 40%, 50%, 60%, 70%, 80% or 90%, or a range defined by any two of the preceding values, optionally 10%-90%, 10%-50%, 30%-80%, 50%-90%, of a typical and/or publicly disclosed concentration of the recombinant adenoviral vector or helper-dependent adenoviral vector for administration by the same route of administration to the same species of animal for treatment of the same or similar disease or disorder.

In some embodiments, in the pharmaceutical composition of any one of embodiments disclosed herein the concentration of the corticosteroid is sufficient to improve one or more of transduction, expression, efficacy and/or safety of the adenoviral-based delivery and expression system when administered to an animal as compared to administration of the same pharmaceutical composition without the corticosteroid. In some embodiments, the improvement includes an improvement in the quantity or duration of a measure of transduction or expression. In some embodiments, the improvement in transfection and/or expression is measured in vitro, for example in a cellular assay. In some embodiments, the improvement in safety includes a reduction in the number, severity or duration of an adverse event, optionally wherein the adverse event is selected from pain, swelling, effusion, inflammation, headache, fever, chills, and flu-like symptoms. In some embodiments, the improvement in efficacy includes an improvement in the onset, amount and/or duration of a therapeutic effect of the adenoviral-based delivery and expression system. In some embodiments, the therapeutic effect includes a reduction in a measure of a symptom or marker of a disease or disorder. In some embodiments, the symptom is pain in a joint of the animal. In some embodiments, the pain is a self-reported measure of pain. In some embodiments, the pain is measured using the Western Ontario and McMaster Universities Osteoarthritis Index pain score (WOMAC-A). In some embodiments, the improvement is, or is at least, 10%, 20%, 30%, 40%, 50%, 60%, 70%, 80%, 90%, 100%, 125%, 150%, 175%, 200%, 300%, 400%, 500%, 1000%, or 5000%, or a range defined by any two of the preceding values, optionally 10%-5000%, 10%-100%, 100%-5000%, or 50%-500%, as compared to the same pharmaceutical composition without the corticosteroid.

In some embodiments, in the pharmaceutical composition of any one of embodiments disclosed herein the concentration of the corticosteroid is, is at least, is not more than, 0.001, 0.01, 0.1, 1.0, 5.0, 10, 15, 20, 25, 30, 40, 50, 60, 70, 80, 90, 100, 250, 500, 750, 1000, 2500, or 5000, or a range defined by any two of the preceding values, optionally 0.001-5000, 0.001-0.1, 0.001-10, 0.1-100, 1.0-100, 1.0-50, 30-300, 25-60, 30-50, 100-1000, 500-5000 mg/ml, optionally 40 mg/ml, of the pharmaceutical composition. In some embodiments, the pharmaceutical composition is a liquid having a volume that is, is at least, or is not more than, 0.1, 0.25, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10 ml, or a range defined by any two of the preceding values, optionally 0.1-10, 0.1-5, 1.0-10, 1.0-5.0, 2.5-7.5, or 4.0-6.0 ml, optionally 1.0, 2.0, 2.5 or 5.0 ml, optionally 1.0 ml.

In some embodiments, in the pharmaceutical composition of any one of embodiments disclosed herein the corticosteroid is in a separate dosage form from the adenoviral-based biological delivery and expression system such that the corticosteroid can be administered before or after the adenoviral-based biological delivery and expression system.

In some embodiments, in the pharmaceutical composition of any one of embodiments disclosed herein the corticosteroid is in the same dosage form as the adenoviral-based biological delivery and expression system such that the corticosteroid is administered simultaneously with the adenoviral-based biological delivery and expression system, optionally wherein the dosage form is a single fluid. In some embodiments, the dosage form including the corticosteroid and the adenoviral-based biological delivery and expression system is prepared by combining a composition including the corticosteroid and a composition including the adenoviral-based biological delivery and expression system.

### Methods

In some embodiments, a method includes administering to an animal the pharmaceutical composition of any one of the embodiments disclosed herein. In some embodiments, the administration of the pharmaceutical composition infects cells of the animal with the adenoviral-based delivery and expression system, resulting in expression of the protein encoded by the nucleic acid of adenoviral-based delivery and expression system. In some embodiments, the administration of the pharmaceutical composition infects joint cells of an osteoarthritis-affected joint of a human in need thereof. In some embodiments, the joint is a knee joint. In some embodiments, the concentration of the corticosteroid in the administered pharmaceutical composition is sufficient to improve one or more of transduction, expression, efficacy and/or safety of the adenoviral-based delivery and expression system when administered to the animal as compared to administration of the same pharmaceutical composition without the corticosteroid. In some embodiments, the improvement includes an improvement in the quantity or duration of a measure of transduction or expression. In some embodiments, the improvement in transfection and/or expression is measured in vitro. In some embodiments, the improvement in safety includes a reduction in the number, severity or duration of an adverse event, optionally wherein the adverse event is selected from pain, swelling, effusion, inflammation, headache, fever, chills, and flu-like symptoms. In some embodiments, the improvement in efficacy includes an improvement in the onset, amount or duration of a therapeutic effect of the adenoviral-based delivery and expression system. In some embodiments, the therapeutic effect includes a reduction in a measure of a symptom or marker of a disease or disorder; optionally wherein the symptom is pain in a joint of the animal, optionally as measured using the Western Ontario and McMaster Universities Osteoarthritis Index pain score (WOMAC-A). In some embodiments, the improvement in efficacy includes an improvement in the onset and/or amount of reduction in a measure of joint pain in the animal. In some embodiments, the improvement in efficacy includes an improvement in the likelihood that the animal will experience at least a 50% reduction in a measure of joint pain. In some embodiments, the improvement is, or is at least, 10%, 20%, 30%, 40%, 50%, 60%, 70%, 80%, 90%, 100%, 125%, 150%, 175%, 200%, 300%, 400%, 500%, 1000%, or 5000%, or a range defined by any two of the preceding values, optionally 10%-5000%, 10%-100%, 100%-5000%, or 50%-500%, as compared to administration of the same pharmaceutical composition without the corticosteroid.

In some embodiments, in the method of any one of embodiments disclosed herein the corticosteroid is administered to the animal a period of time that is, or is not more than 0.00, 0.01, 0.05, 0.1, 0.2, 0.3, 0.4, 0.5, 1, 1.5, 2, 2.5, 3, 4, or 5 hours, or a range defined by any two of the preceding values, optionally 0.0-5, 0.0-3, 0.0-2, 0.0-1, 0.0-0.5, 0.0-0.2, or 0.0-0.01 hours prior to the administration of the adenoviral-based delivery and expression system. In some embodiments, the corticosteroid is administered to the animal a period of time that is, or is not more than 0.00, 0.01, 0.05, 0.1, 0.2, 0.3, 0.4, 0.5, 1, 1.5, 2, 2.5, 3, 4, or 5 hours, or a range defined by any two of the preceding values, optionally 0.0-5, 0.0-3, 0.0-2, 0.0-1, 0.0-0.5, 0.0-0.2, or 0.0-0.01 hours after the administration of the adenoviral-based delivery and expression system. In some embodiments, the corticosteroid is administered to the animal not more than 3 hours before and not more than 30 minutes after the administration of the adenoviral-based delivery and expression system. In some embodiments, the corticosteroid is administered to the animal not more than 30 minutes before and not more than 30 minutes after the administration of the adenoviral-based delivery and expression system. In some embodiments, the corticosteroid is administered to the animal not more than 5 minutes before and not more than 5 minutes after the administration of the adenoviral-based delivery and expression system. In some embodiments, the corticosteroid is administered to the animal simultaneously with the adenoviral-based delivery and expression system. In some embodiments, the corticosteroid and the adenoviral-based delivery and expression system are in a single dosage form.

In some embodiments, in the method of any one of embodiments disclosed herein the animal has not been administered a corticosteroid systemically and/or locally, other than the corticosteroid of the pharmaceutical composition, for a period of time that is, or is at least, 2, 3, 4, 5, 6, 12, 18, 24, 48, or 72 hours, or a range defined by any two of the preceding values. In some embodiments, the animal has not been administered a corticosteroid, other than the corticosteroid of the pharmaceutical composition, for a period of time that is, or is at least, 2-72, 3-72, 4-72, 5-72, 2-24, 3-24, 5-24, 2-48, 3-48, 4-48, 5-48, 6-72, 6-48, 12-72, or 18-72 hours prior to administration of the adenoviral-based delivery and expression system.

In some embodiments, in the method of any one of embodiments disclosed herein the method includes administration of the corticosteroid of the pharmaceutical composition systemically and administration of the adenoviral-based delivery and expression system of the pharmaceutical composition locally. In some embodiments, the method includes administration of the corticosteroid and adenoviral-based delivery and expression system of the pharmaceutical composition locally to the same location of the animal. In some embodiments, the method includes administration of the corticosteroid and adenoviral-based delivery and expression system of the pharmaceutical composition topically, by injection, by inhalation, intra-ocularly, or intra-ear. In some embodiments, the method includes administration of the corticosteroid and adenoviral-based delivery and expression system of the pharmaceutical composition by injection, wherein the administration of the corticosteroid and the adenoviral-based delivery and expression system is through the same needle and the needle is maintained in the same site during administration of both the corticosteroid and the adenoviral-based delivery and expression system. In some embodiments, the needle is flushed with a fluid, for example saline, between administration of the corticosteroid and the adenoviral-based delivery and expression system.

In some embodiments, in the method of any one of embodiments disclosed herein the method includes injection into a joint of the animal, optionally intra-tendinous, intra-muscular, intra-articular, or sub-acromial injection. In some embodiments, the injection is an intra-articular injection. In some embodiments, the joint is the knee.

In some embodiments, in the method of any one of embodiments disclosed herein the method includes injection into an intervertebral disc. In some embodiments, the injection is intradiscal injection. In some embodiments, the intradiscal injection is into the nucleus pulposus (NP) region of the intervertebral disc.

In some embodiments, in the method of any one of embodiments disclosed herein the method does not include administration into an intervertebral disc. In some embodiments, in the method of any one of embodiments disclosed herein the method does not include administration by intradiscal injection. In some embodiments, in the method of any one of embodiments disclosed herein the method does not include intradiscal injection into the nucleus pulposus (NP) or central gelatinous nucleus pulposus (NP) region of the intervertebral disc.

In some embodiments, the method does not comprise expressing IL-IRA in cells of one or more intervertebral discs of a subject suffering from degenerative disc disease (DDD) or a condition associated with DDD, wherein the method comprises: a) infecting cells of one or more intervertebral discs of the subject in need thereof with a pharmaceutical composition comprising an adenoviral-based biological delivery and expression system comprising a nucleic acid encoding a interleukin-1 receptor antagonist (IL-1Ra) protein; and b) expressing IL-1Ra in the cells of the one or more intervertebral discs. In some embodiments, the method does not comprise administration of a corticosteroid and/or a local anesthetic into an intervertebral disc of a subject. In some of embodiments, the method dose not comprise a method wherein a corticosteroid and/or local anesthetic is in a single pharmaceutical formulation with the adenoviral-based biological delivery and expression system such that the corticosteroid and/or local anesthetic are administered into an intervertebral disc of the subject simultaneously with the adenoviral-based biological delivery and expression system. In some embodiments, the method does not comprise a method wherein the corticosteroid and/or local anesthetic is not in a single pharmaceutical formulation with the adenoviral-based biological delivery and expression system such that the corticosteroid and/or local anesthetic are administered into an intervertebral disc of the subject before and/or after the adenoviral-based biological delivery and expression system. In some embodiments, the method does not comprise intradiscal injection of a corticosteroid and/or local anesthetic and/or fluid.

In some embodiments, the method does not further comprise administering a corticosteroid, for example methylprednisolone, betamethasone, triamcinolone, or combinations thereof, into the intervertebral disc of a subject. In some embodiments, the method does not comprise the corticosteroid formulated in a single pharmaceutical composition with the adenoviral-based biological delivery and expression system such that the corticosteroid is administered into the intervertebral disc of a subject simultaneously with the adenoviral-based biological delivery and expression system. In some embodiments, the method does not comprise the corticosteroid in a separate dosage form from the adenoviral-based biological delivery and expression system, such that the corticosteroid can be administered into the intervertebral disc of a subject prior to or after the administration of the adenoviral-based biological delivery and expression system to an intervertebral disc of a subject. In some embodiments, the method does not further comprise administering both a corticosteroid and a local anesthetic to an intervertebral disc of a subject. In some embodiments, the method dose not comprise one or both of the corticosteroid and the local anesthetic formulated in a single pharmaceutical composition with the adenoviral-based biological delivery and expression system such that the corticosteroid and/or local anesthetic are administered into the intervertebral disc of a subject simultaneously with the adenoviral-based biological delivery and expression system. In some embodiments, the method does not comprise one or both of the corticosteroid and local anesthetic in separate dosage form(s) from the adenoviral-based biological delivery and expression system, such that the corticosteroid and/or local anesthetic can be administered prior to or after the administration of the adenoviral-based biological delivery and expression system to an intervertebral disc of a subject.

In some embodiments, in the method of any one of embodiments disclosed herein the method includes treating a disease or condition. In some embodiments, the method includes identifying the animal as suffering from or at risk of suffering from the treated disease or condition prior to administration of the pharmaceutical composition. In some embodiments, the disease or condition is: camptodactyly-arthropathy-coxa vara-pericarditis (CACP) syndrome; a musculoskeletal disease or condition; a joint disease or condition; a lung disease or condition; an eye disease or condition; an ear disease or condition; arthropathies, arthritis, or arthritis-related conditions; osteoarthritis, rheumatoid arthritis, gout and pseudo-gout, septic arthritis, psoriatic arthritis, ankylosing spondylitis, juvenile idiopathic arthritis, Still's disease, Reiter's syndrome, or tendinopathies including tendonitis, tendinosis, and tenosynovitis; synovial disorders including synovitis; a bursa disorder, including bursitis; or equine musculoskeletal disorders including bone spavin, navicular syndrome, and osselet. In some embodiments, the disease or condition is degenerative disc disease (DDD) or a condition associated with DDD.

In some embodiments, in the method of any one of embodiments disclosed herein the method includes treating a disease or condition, and the disease or condition is not degenerative disc disease (DDD) or a condition associated with DDD.

In some embodiments, in the method of any one of embodiments disclosed herein the method includes treating a disease or condition, and the disease or condition is osteoarthritis or an osteoarthritic condition, optionally of the knee. In some embodiments, the animal is a mammal. In some embodiments, the animal is a human. In some embodiments, the animal is a horse, a dog or a cat. In some embodiments, the adenoviral-based delivery and expression system is a recombinant adenoviral vector or a helper-dependent adenoviral vector, and the concentration of vector is, is at least, or is not more than, 1.4×10⁵, 1.4×10⁶, 2.8×10⁶, 7.0×10⁶, 1.4×10⁷, 2.8×10⁷, 7.0×10⁷, 1.4×10⁸, 2.8×10⁸, 7.0×10⁸, 1.4×10⁹, 2.8×10⁹, 7.0×10⁹, 1.4×10¹⁰, 2.8×10¹⁰, 7.0×10¹⁰, 1.4×10¹¹, 2.8×10¹¹, 7.0×10¹¹, 1.4×10¹², 2.8×10¹², 7.0×10¹², 1.4×10¹³, 2.8×10¹³, 7.0×10¹³ or a range defined by any two of the preceding values, optionally 1.4×10⁵ to 7.0×10¹³, 1.4×10⁵ to 7.0×10¹², 1.4×10⁵ to 7.0×10¹¹, 1.4×10⁵ to 7.0×10¹⁰, 1.4×10⁵ to 7.0×10⁹, 1.4×10⁵ to 7.0×10⁸, 1.4×10⁹ to 7.0×10¹⁰, optionally 2.8×10⁹, 2.8×10¹⁰, 2.8×10¹¹, genome copies (GC) of recombinant adenoviral vector or a helper-dependent adenoviral vector per ml of the pharmaceutical composition. In some embodiments, the amount of vector administered to the animal is reduced to 10%, 20%, 30%, 40%, 50%, 60%, 70%, 80% or 90%, or a range defined by any two of the preceding values, optionally 10%-90%, 10%-50%, 30%-80%, 50%-90%, of a typical and/or publicly disclosed amount of the recombinant adenoviral vector or a helper-dependent adenoviral vector administered by the same route of administration to the same species of animal for treatment of the same or similar disease or disorder. In some embodiments, the adenoviral-based delivery and expression system is a recombinant adenoviral vector or a helper-dependent adenoviral vector, and the concentration of vector is, is at least, or is not more than, 1.4×10⁵, 1.4×10⁶, 2.8×10⁶, 7.0×10⁶, 1.4×10⁷, 2.8×10⁷, 7.0×10⁷, 1.4×10⁸, 2.8×10⁸, 7.0×10⁸, 1.4×10⁹, 2.8×10⁹, 7.0×10⁹, 1.4×10¹⁰, 2.8×10¹⁰, 7.0×10¹⁰, 1.4×10¹¹, 2.8×10¹¹, 7.0×10¹¹, 1.4×10¹², 2.8×10¹², 7.0×10¹², 1.4×10¹³, 2.8×10¹³, 7.0×10¹³ or a range defined by any two of the preceding values, optionally 1.4×10⁵ to 7.0×10¹³, 1.4×10⁵ to 7.0×10¹², 1.4×10⁵ to 7.0×10¹¹, 1.4×10⁵ to 7.0×10¹⁰, 1.4×10⁵ to 7.0×10⁹, 1.4×10⁵ to 7.0×10⁸, 1.4×10⁹ to 7.0×10¹⁰, viral particles (VP) of recombinant adenoviral vector or a helper-dependent adenoviral vector per ml of the pharmaceutical composition. In some embodiments, the pharmaceutical composition is a liquid having a volume that is, is at least, or is not more than, 0.1, 0.25, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10 ml, or a range defined by any two of the preceding values, optionally 0.1-10, 0.1-5, 1.0-10, 1.0-5.0, 2.5-7.5, or 4.0-6.0 ml, optionally 2.0, 2.5 or 5.0 ml, optionally 5.0 ml. In some embodiments, the pharmaceutical composition is a liquid, and wherein the amount pharmaceutical composition administered to the animal is, is at least, or is not more than, 0.1, 0.25, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10 ml, or a range defined by any two of the preceding values, optionally 0.1-10, 0.1-5, 1.0-10, 1.0-5.0, 2.5-7.5, or 4.0-6.0 ml, optionally 1.0, 2.0, 2.5 or 5.0 ml, optionally 5.0 ml. In some embodiments, the adenoviral-based delivery and expression system is a recombinant adenoviral vector or a helper-dependent adenoviral vector, and the amount of vector administered to the animal is, is at least, or is not more than, 1.4×10⁵, 1.4×10⁶, 2.8×10⁶, 7.0×10⁶, 1.4×10⁷, 2.8×10⁷, 7.0×10⁷, 1.4×10⁸, 2.8×10⁸, 7.0×10⁸, 1.4×10⁹, 2.8×10⁹, 7.0×10⁹, 1.4×10¹⁰, 2.8×10¹⁰, 7.0×10¹⁰, 1.4×10¹¹, 2.8×10¹¹, 7.0×10¹¹, 1.4×10¹², 2.8×10¹², 7.0×10¹², 1.4×10¹³, 2.8×10¹³, 7.0×10¹³ or a range defined by any two of the preceding values, optionally 1.4×10⁵ to 7.0×10¹³. 1.4×10⁵ to 7.0×10¹², 1.4×10⁵ to 7.0×10¹¹, 1.4×10⁵ to 7.0×10¹⁰, 1.4×10⁵ to 7.0×10⁹, 1.4×10⁵ to 7.0×10⁸, 1.4×10⁹ to 7.0×10¹⁰, optionally 1.4×10⁵, 1.4×10⁷, 5.6×10⁹, 7.0×10⁹, 1.4×10¹⁰, 1.4×10¹¹, or 1.4×10¹², optionally 5.6×10⁹, 7.0×10⁹, 1.4×10¹⁰, 1.4×10¹¹, or 1.4×10¹², genome copies (GC) of recombinant adenoviral vector or a helper-dependent adenoviral vector. In some embodiments, the adenoviral-based delivery and expression system is a recombinant adenoviral vector or a helper-dependent adenoviral vector, and the amount of vector administered to the animal is, is at least, or is not more than, 1.4×10⁵, 1.4×10⁶, 2.8×10⁶, 7.0×10⁶, 1.4×10⁷, 2.8×10⁷, 7.0×10⁷, 1.4×10⁸, 2.8×10⁸, 7.0×10⁸, 1.4×10⁹, 2.8×10⁹, 7.0×10⁹, 1.4×10¹⁰, 2.8×10¹⁰, 7.0×10¹⁰, 1.4×10¹¹, 2.8×10¹¹, 7.0×10¹¹, 1.4×10¹², 2.8×10¹², 7.0×10¹², 1.4×10¹³, 2.8×10¹³, 7.0×10¹³ or a range defined by any two of the preceding values, optionally 1.4×10⁵ to 7.0×10¹³, 1.4×10⁵ to 7.0×10¹², 1.4×10⁵ to 7.0×10¹¹, 1.4×10⁵ to 7.0×10¹⁰, 1.4×10⁵ to 7.0×10⁹, 1.4×10⁵ to 7.0×10⁸, 1.4×10⁹ to 7.0×10¹⁰, optionally 1.4×10⁵, 1.4×10⁷, 5.6×10⁹, 7.0×10⁹, 1.4×10¹⁰, 1.4×10¹¹, or 1.4×10¹², optionally 5.6×10⁹, 7.0×10⁹, 1.4×10¹⁰, 1.4×10¹¹, or 1.4×10¹², viral particles (VP) of recombinant adenoviral vector or a helper-dependent adenoviral vector. In some embodiments, the concentration of the corticosteroid is, is at least, is not more than, 0.001, 0.01, 0.1, 1.0, 5.0, 10, 15, 20, 25, 30, 40, 50, 60, 70, 80, 90, 100, 250, 500, 750, 1000, 2500, or 5000, or a range defined by any two of the preceding values, optionally 0.001-5000, 0.001-0.1, 0.001-10, 0.1-100, 1.0-100, 1.0-50, 30-300, 25-60, 30-50, 100-1000, 500-5000 mg/ml, optionally 40 mg/ml, of the pharmaceutical composition. In some embodiments, the pharmaceutical composition is a liquid having a volume that is, is at least, or is not more than, 0.1, 0.25, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10 ml, or a range defined by any two of the preceding values, optionally 0.1-10, 0.1-5, 1.0-10, 1.0-5.0, 2.5-7.5, or 4.0-6.0 ml, optionally 1.0, 2.0, 2.5 or 5.0 ml, optionally 1.0 ml. In some embodiments, the pharmaceutical composition is a liquid and wherein the amount pharmaceutical composition administered to the animal is, is at least, or is not more than, 0.1, 0.25, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10 ml, or a range defined by any two of the preceding values, optionally 0.1-10, 0.1-5, 1.0-10, 1.0-5.0, 2.5-7.5, or 4.0-6.0 ml, optionally 1.0, 2.0, 2.5 or 5.0 ml, optionally 1.0 ml. In some embodiments, the amount of corticosteroid administered to the animal is, is at least, is not more than, 0.001, 0.01, 0.1, 1.0, 5.0, 10, 15, 20, 25, 30, 40, 50, 60, 70, 80, 90, 100, 250, 500, 750, 1000, 2500, or 5000, or a range defined by any two of the preceding values, optionally 0.001-5000, 0.001-0.1, 0.001-10, 0.1-100, 1.0-100, 1.0-50, 30-300, 25-60, 30-50, 100-1000, 500-5000 mg, optionally 40 mg.

In some embodiments, in the method of any one of embodiments disclosed herein the method includes: identifying a human subject as suffering from osteoarthritis of the knee, administering the pharmaceutical composition to the human subject by intra-articular injection into an osteoarthritis-affected knee joint, where the corticosteroid is in a separate dosage form from the adenoviral-based biological delivery and expression system, where administration of the corticosteroid includes intra-articular injection of 1 ml of a dosage form including 40 mg/ml of the corticosteroid, where administration of the adenoviral-based biological delivery and expression system includes intra-articular injection of 2, 2.5 or 5 ml of a dosage form including optionally 2.8×10⁹, 2.8×10¹⁰, 2.8×10¹¹ per ml of the adenoviral-based biological delivery and expression system, where the amount of adenoviral-based biological delivery and expression system delivered to the knee is 5.6×10⁹, 7.0×10⁹, 1.4×10¹⁰, 1.4×10¹¹, or 1.4×10¹², genome copies (GC) of recombinant adenoviral vector or a helper-dependent adenoviral vector, where the administration of the corticosteroid and the adenoviral-based delivery and expression system is through the same needle and the needle is maintained in the same site during administration of both the corticosteroid and the adenoviral-based delivery and expression system, optionally where the needle is flushed with a fluid between corticosteroid and the adenoviral-based delivery and expression system, and where the corticosteroid is administered not more than 0-15 minutes before administration of the adenoviral-based delivery and expression system. In some embodiments, the corticosteroid is administered not more than 0-5 minutes before administration of the adenoviral-based delivery and expression system. In some embodiments, the adenoviral-based delivery and expression system includes a helper-dependent adenoviral vector including a nucleic acid sequence encoding human IL-1Ra protein, left and right inverted terminal repeats, an adenoviral packaging signal and non-viral, and non-coding stuffer nucleic acid sequences, wherein the expression of the nucleic acid sequence encoding human IL-1Ra protein is regulated by a NF-κB inducible promoter, which is located upstream of the reading frame of the nucleic acid sequence encoding the human IL-1Ra protein. In some embodiments, the genome of helper-dependent adenoviral vector has nucleic acid sequence of SEQ ID NO: 7. In some embodiments, the corticosteroid is methylprednisolone.

### Uses and Kits

In some embodiments, a kit includes the pharmaceutical composition of any one of embodiments disclosed herein where the corticosteroid is in a separate dosage form from the adenoviral-based biological delivery and expression system, where the kit includes a first container including the corticosteroid in a first dosage form, and a second container including the adenoviral-based biological delivery and expression system in a second dosage form.

In some embodiments, a kit for use in preparing the pharmaceutical composition of any one of the embodiments disclosed herein where the corticosteroid is in the same dosage form as the adenoviral-based biological delivery and expression system, the kit includes the composition including the corticosteroid in a first container and the composition including the adenoviral-based biological delivery and expression system in a second container.

In some embodiments, a composition or kit of any one of embodiments disclosed herein is for use in a method of treating a disease or condition, the method including the method disclosed in any one of the embodiments disclosed herein. In some embodiments, a kit includes a first container including a corticosteroid in a first dosage form, and a second container including an adenoviral-based biological delivery and expression system in a second dosage form. In some embodiments, the corticosteroid or the adenoviral-based biological delivery and expression system is provided as disclosed in any embodiment disclosed herein. In some embodiments, a pharmaceutical composition including a corticosteroid and an adenoviral-based delivery and expression system according to any one of the embodiments disclosed herein is for use as a medicament. In some embodiments, a kit including a first container including a corticosteroid in a first dosage form, and a second container including an adenoviral-based biological delivery and expression system in a second dosage form according to any one of embodiments disclosed herein is for use as a medicament. In some embodiments, a pharmaceutical composition including a corticosteroid and an adenoviral-based delivery and expression system according to any one of the embodiments disclosed herein is for use in the prevention or treatment of a disease or condition. In some embodiments, a kit including a first container including a corticosteroid in a first dosage form, and a second container including an adenoviral-based biological delivery and expression system in a second dosage form according to any one of the embodiments disclosed herein is for use in the prevention or treatment of a disease or condition. In some embodiments, the disease of condition is selected from the group consisting of: camptodactyly-arthropathy-coxa vara-pericarditis (CACP) syndrome; a musculoskeletal disease or condition; a joint disease or condition; a lung disease or condition; an eye disease or condition; an ear disease or condition; arthropathies, arthritis, and arthritis-related conditions; osteoarthritis, rheumatoid arthritis, gout and pseudo-gout, septic arthritis, psoriatic arthritis, ankylosing spondylitis, juvenile idiopathic arthritis, Still's disease, Reiter's syndrome, or tendinopathies including tendonitis, tendinosis, and tenosynovitis; synovial disorders including synovitis; a bursa disorder, including bursitis; or equine musculoskeletal disorders including bone spavin, navicular syndrome, and osselet. In some embodiments, the disease or condition is degenerative disc disease (DDD) or a condition associated with DDD. In some embodiments, the disease or condition is not degenerative disc disease (DDD) or a condition associated with DDD.

### PCRX201

In some embodiments, in the pharmaceutical composition, kit, method or use according to any one of the embodiments disclosed herein, the adenoviral-based biological delivery and expression system is PCRX201. In some embodiments, in the pharmaceutical composition, kit, method or use according to any one of the embodiments disclosed herein, the adenoviral-based biological delivery and expression system is a helper-dependent adenovirus, wherein the genome consists of a nucleic acid of SEQ ID NO: 7.

In some embodiments, in the pharmaceutical composition, kit, method or use according to any one of the embodiments disclosed herein, the adenoviral-based biological delivery and expression system provides for a long-term expression of IL-1Ra in cells at human joints for the treatment and prevention of osteoarthritis. In some embodiments, the adenoviral-based biological delivery and expression system for IL-1Ra gene therapy is PCRX201, (humantakinogene hadenovec, also referred to herein as FX201 or PCRX-201), for intra-articular (IA) administration that is being developed for the treatment of patients' osteoarthritis or osteoarthritic conditions. PCRX201, is a helper-dependent adenovirus (HDAd) delivering the nucleic acid sequence encoding the human IL-1Ra under the control of a nuclear factor-κB (NF-κB)-inducible promoter for IA administration to patients with osteoarthritis or osteoarthritic condition. Following IA injection, PCRX201 infects cells in the joint to produce IL-1Ra locally in response to inflammation. PCRX201 is a non-replicating, non-integrating HDAd vector with no viral coding sequences that has been engineered to carry the genetic coding sequence for IL-1Ra. Only the adenoviral packaging signal and inverted terminal repeats (ITRs) remain in the PCRX201 genome as they are required for manufacturing. Transcription is controlled by the inflammation-sensitive NF-κB-inducible promoter, which drives expression of IL-1Ra in response to an inflammatory environment.

PCRX201 can be administered as a single dose by IA injection. The expected clinical benefits are sustained symptomatic relief, including both reduction in pain and improvement or restoration of function, and a beneficial modification of the underlying disease process in patients with osteoarthritis or an osteoarthritic condition in a human joint. Advantageously, the adenoviral delivery and expression systems of the present disclosure specifically locates in the joints when administered intra-articularly. Most importantly, no measurable concentration of vector sequences could be detected in the liver of mice treated with the adenoviral system of the disclosure. Therefore, IL-1Ra concentrations are expected to be highest in the joints injected with the vector of the disclosure while no significant side effects are expected in any other organ. Described below are the properties of PCRX201. The administration of a corticosteroid as disclosed herein improves one or more measures of the efficacy and/or safety of PCRX201.

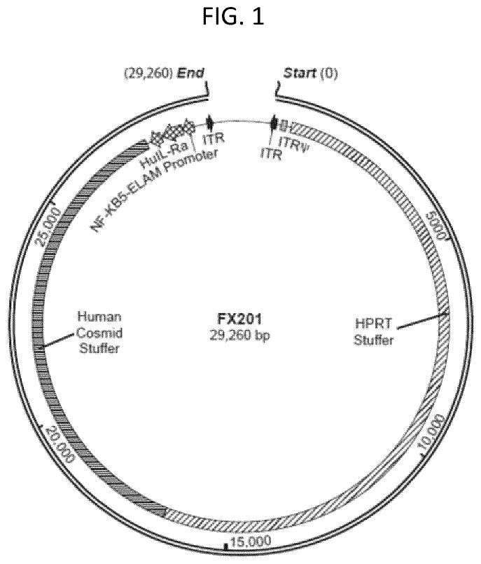

Vector Backbone: PCRX201 is a non-replicating, non-integrating HDAd vector. The genomic component is composed of double-stranded linear DNA approximately 29.3 kilobases (kb) in size. The annotated sequence obtained by next-generation sequencing confirms the key elements in the PCRX201 genome. The PCRX201 genome contains minimal adenoviral elements required for amplification and packaging to allow for its manufacturing: left and right inverted terminal repeats (hereafter referred to as “L ITR” and “R ITR”, respectively) and the packaging signal (Y). Approximately 1.1 kb of the PCRX201 genome is composed of the nucleic acid sequence encoding human IL-1Ra, which is inserted on the right end of the genome in reverse (right-to-left) orientation, and the promoter, placed just before the R ITR. The promoter is 5 species-conserved NF-κB binding motif repeats fused to a proximal promoter region of the human ELAM gene, responding to pro-inflammatory cytokines (Schindler 1994). Approximately 27 kb of the PCRX201 genome consists of non-coding stuffer sequence composed of human hypoxanthine phosphoribosyltransferase (HPRT) and human cosmid insert, which is inserted to enlarge the PCRX201 genome to a size which allows efficient packaging of the vector genome into each viral particle. An embodiment of a genome map for PCRX201 is presented in FIG. 1.

Gene of Interest: The PCRX201 genome contains a 534 base pair (bp) sequence of human IL-1Ra, which is regulated by a 262 bp sequence of NF-κB-inducible promoter.

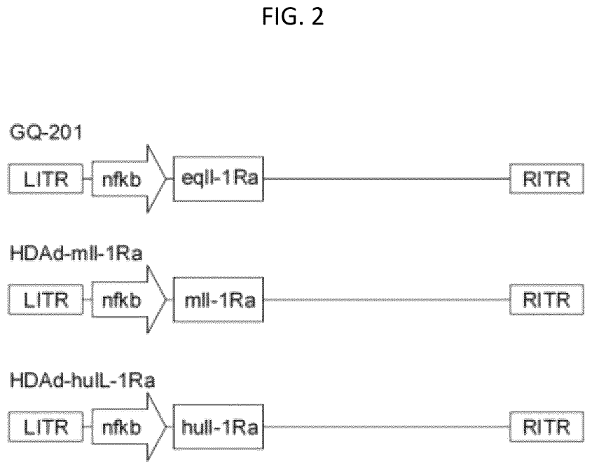

Disclosed herein are gene maps of embodiments of the PCRX201, HDAd vectors of the disclosure (FIG. 2). All three vectors contain the inflammation-sensitive NF-κB5-ELAM promoter upstream of the IL-1Ra cDNA according to any one of SEQ ID NOs: 1 or 4, as well as ITR and an adenoviral packaging signal. The full vector sequence of GQ-201, HDAd-mIL-Ra and HDAd-human IL-1Ra, is shown in SEQ ID NOs: 2, 3 and 7 respectively. The only difference between the three vectors is that GQ-201 carries the equine variant of IL-1Ra, HDAd-mIL-Ra has the murine IL-1Ra variant and HDAd-huIL-1Ra carries the human IL-1Ra. As an example, the HDAd-mIL-Ra of nucleic acid sequence according to SEQ ID NO: 3 can comprise a nucleic acid encoding a murine IL-1Ra according to SEQ ID NO: 1. As an example, the HDAd-mIL-Ra of nucleic acid sequence according to SEQ ID NO: 7 can comprise a nucleic acid encoding a murine IL-1Ra according to SEQ ID NO: 4. The vectors can be prepared my methods known in the art, for example as disclosed in WO 2021/0055860, which is incorporated herein by reference for this disclosure, and its entirety.

In some embodiments, the helper-dependent adenoviral vectors of the present disclosure, e.g., PCRX201, do not carry any viral sequences, except for the L ITR, R ITR and the adenoviral packaging signal. Exemplary helper-dependent adenoviral vectors to be used in embodiments of the present disclosure are those based on the helper virus and helper-dependent backbone system developed by Palmer and Ng (Palmer, D., and Ng, P. (2003). Improved system for helper-dependent adenoviral vector production. Mol Ther 8, 846-852.) and Toietta et al (Toietta, G., Pastore, L., Cerullo, V., Finegold, M., Beaudet, A. L., and Lee, B. (2002). Generation of helper-dependent adenoviral vectors by homologous recombination. Mol Ther 5, 204-210). In some embodiments, the adenoviral delivery and expression system according to the present disclosure can comprise a nucleic acid sequence of the adenoviral-based biological delivery and expression system comprising the promoter, the nucleic acid sequence encoding the IL-1Ra, the left and the right inverted terminal repeats, the adenoviral packaging signal and the non-viral, non-coding stuffer nucleic acid sequences as set forth in SEQ ID NO: 2, SEQ ID NO: 3 or SEQ ID NO: 7, or a biologically effective part thereof. The nucleic acid sequence of SEQ ID NO 2 describes a murine helper-dependent adenoviral vector, the sequence set forth in SEQ ID NO 3 describes an equine helper-dependent adenoviral vector, the sequence set forth in SEQ ID NO 7 describes a human helper-dependent adenoviral vector, all three vectors bearing any one of a murine, an equine IL-1Ra gene or human IL-1Ra gene respectively. In some embodiments, the system of the disclosure has any one of at least 96%, 97%, 98%, or 99% sequence identity with the vector set forth in SEQ ID NO: 2, SEQ ID NO: 3 or SEQ ID NO: 7.

“Long term expression” in the context of the present disclosure has its ordinary and customary meaning as read in light of this disclosure, and includes the meaning that the gene product of the adenoviral delivery and expression system (e.g., IL-1Ra), is expressed in the joint(s) infected with the helper-dependent adenoviral vector of the disclosure, for at least 3 months, 6 months or 12 months. In some embodiments, IL-1Ra is expressed in the joint(s) infected with the helper-dependent adenoviral vector of the disclosure for at least 3 months.

In some embodiments, the helper-dependent adenoviral vector of the disclosure comprises a nucleic acid sequence encoding IL-1Ra under control of an inflammation-sensitive promoter. Although IL-1Ra contains species-specific nucleic acid sequences, the adenoviral vector is able to express interleukin-1 receptor antagonist (IL-1Ra) from any mammalian species or human. In some embodiments, the cDNA of the mammalian interleukin-1 receptor antagonist (IL-1Ra) used for cloning is a cDNA selected from the group consisting of human IL-1Ra, murine IL-1Ra, equine IL-1Ra, canine IL-1Ra, cat II-1Ra, rabbit IL-1Ra, hamster IL-1Ra, bovine IL-1Ra, camel IL-1Ra or their homologs in other mammalian species.

In some embodiments, in order to monitor the presence of genomic vector sequences in synovial cells, the helper-dependent adenoviral vector according to the disclosure can further comprise a sequence encoding a marker gene that is visually or instrumentally detectable. In some embodiments, the marker gene is green fluorescence protein (GFP), LacZ, or luciferase enzyme.

In some embodiments, the nucleic acid sequence of human IL-1Ra used in the present disclosure is shown in the sequence listing set forth in SEQ ID NO: 4. As noted herein, in some embodiments a nucleic acid sequence resulting in a biologically active IL-1Ra protein of a human is used in the context of the present disclosure. In some embodiments, a conserved nucleic acid sequences encoding for the same amino acids, polypeptide or protein is used in an embodiment of the present disclosure. In some embodiments, the helper-dependent adenoviral vector according to the disclosure contains a nucleic acid sequence (e.g. cDNA) of IL-1Ra having at least 95%, 96%, 97%, 98% or 99% sequence homology with the nucleic acid sequence shown in SEQ ID NO: 4. In some embodiments, the nucleic acid sequence encodes a human IL-1Ra protein of amino acid sequence that is, or is at least 95%, 96%, 97%, 98%, 99% or 100% identical to SEQ ID NO: 6.

In some embodiments, a pharmaceutical composition including a corticosteroid and an adenoviral-based biological delivery and expression system is provided for treatment of osteoarthritis or an osteoarthritic condition in a human joint or for the prevention of such conditions in a human identified to be at risk of developing osteoarthritis or an osteoarthritic condition. In some embodiments, the adenoviral-based biological delivery and expression system is PCRX201. In some embodiments, the adenoviral-based biological delivery and expression system comprises: a) 1.4×10⁸ to 1.4×10¹² GC of the helper-dependent adenoviral vector per milliliter (GC per ml); b) less than 15% helper virus particles; c) less than 10% empty capsids; d) not more than 100 μg/ml of host cell protein; e) not more than 20 ng/ml of host cell nucleic acid; f) not more than 35EU/ml of endotoxin; and g) a Viral Particle to Infectious Unit Ratio of ≤than 300GC/TCID₅₀.

In some embodiments, PCRX201 can be quantifying the vector using Droplet Digital™ polymerase chain reaction (ddPCR). In some embodiments, GC/Dose can be converted to VP/Dose and vice versa using Table 1 below.

TABLE 1
Comparison of VP/Dose and GC/Dose

| | | VP/mL | | |
|---|---|---|---|---|
| | | synovial | | GC per ml |
| | VP/dose^{a} | fluid | GC/dose^{a} | synovial fluid |
| Human Dose 1 | 1 × 10¹⁰ | 2 × 10⁹ | 1.4 × 10¹⁰ | 2.8 × 10⁹ |
| Human Dose 2 | 1 × 10¹¹ | 2 × 10¹⁰ | 1.4 × 10¹¹ | 2.8 × 10¹⁰ |
| Human Dose 3 | 1 × 10¹² | 2 × 10¹¹ | 1.4 × 10¹² | 2.8 × 10¹¹ |

^{a}Assumes a 5 mL injection volume in human (Heilmann 1996, Hansen 2011); VP, Viral particle; GC, Genome copy.

### Pharmaceutical Formulations

In some embodiments, in the pharmaceutical composition of any one of the embodiments disclosed herein the composition is formulated using a method known in the art, for example as disclosed in WO 2021/0055860, which is incorporated herein by reference for this disclosure, and its entirety. Such compositions typically comprise the helper-dependent adenoviral vector viral particles as disclosed herein, a helper adenovirus and a pharmaceutically acceptable carrier. As used herein, the term “pharmaceutically acceptable carrier” has its ordinary and customary meaning as read in light of this disclosure, and may include any solvents, dispersion media, coatings, antibacterial and antifungal agents, isotonic and absorption delaying agents, and the like, compatible with intended route of administration. Suitable carriers are described in the most recent edition of Remington's Pharmaceutical Sciences, a standard reference text in the field, which is incorporated herein by reference in its entirety. In some embodiments, examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated for some embodiments. In some embodiments, supplementary active compounds are also incorporated into the compositions.

In some embodiments, the pharmaceutical composition of any one of the embodiments disclosed herein is formulated to be compatible with its intended route of administration as disclosed herein. Examples of routes of administration, some of which are used in embodiments disclosed herein, include parenteral, e.g., intraarticular, intravenous, intradermal, subcutaneous, oral (e.g., inhalation), transdermal (i.e., topical), transmucosal, and rectal administration. Examples of solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

In some embodiments, pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In some embodiments, the composition is sterile and should be fluid to the extent that easy syringeability exists. In some embodiments, it is stable under the conditions of manufacture and storage and is preserved against the contaminating action of microorganisms such as bacteria and fungi. In some embodiments, the carrier can be a solvent or dispersion medium containing, for example, water, ethanol, polyol (for example, glycerol, propylene glycol, and liquid polyethylene glycol, and the like), and suitable mixtures thereof. In some embodiments, the proper fluidity can be maintained, for example, by the use of a coating such as lecithin, by the maintenance of the required particle size in the case of dispersion and by the use of surfactants. In some embodiments, prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

In some embodiments, sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

### Chemical Properties of the Adenoviral Expression and Delivery System of the Disclosure

In some embodiments, the capsid of the adenoviral-based biological delivery and expression system of any embodiment disclosed herein, e.g. PCRX201, is unenveloped and can include 29.3 kb double-stranded DNA. In some embodiments, the theoretical molecular weight of the capsid can be 103.9 megadaltons (MDa) and the genome can be 18.1 MDa. In some embodiments, the capsid of PCRX201 can have a diameter of approximately 100 nm.

Formulations: In some embodiments, PCRX201 is formulated in a buffer composed of about 1-20 mM, TRIS, about 50-100 mM NaCl, 0.01-1% weight/volume (w/v) Polysorbate 80, 1-10% (w/v) sucrose, 0.1-10 mM MgCl₂, 50-500 μM EDTA, 1-5% volume/volume (v/v) ethanol, and 5-50 mM L-histidine. In some embodiments, PCRX201 can be formulated in a buffer composed of 10 mM TRIS, 75 mM NaCl, 0.02% (weight/volume (w/v) Polysorbate 80, 5% (w/v) sucrose, 1.0 mM MgCl₂, 100 μM EDTA, 0.5% (volume/volume (v/v) of ethanol), and 10 mM L-histidine. In some embodiments, the product can be a clear to slightly opalescent, colorless suspension with no visible particulates. In some embodiments, the formulation further comprises a corticosteroid. In some embodiments, the concentration of the steroid is 5-10, or 8 mg/ml.

Storage Conditions and Stability: In some embodiments, PCRX201 is stored as a frozen liquid at ≤−65° C. In some embodiments, the PCRX201 formulation is stable for at least 3 months, at least 6 months or at least 12 months when stored at ≤−65° C. Once thawed, the product must be stored at 2-8° C. and used within 7 days. PCRX201 may be kept at room temperature (RT) for some period of time. Once a vial is ready for use it can be held at RT in vial for no more than 7 hours (vials held at RT cannot be returned to refrigeration for later use). Once the PCRX201 dosage is prepared in the syringe, it must be held at RT and used within 4 hours.

### Treatment Monitoring

In some embodiments, in a method of any one of the embodiments disclosed herein the method improves one or more of transduction, expression, efficacy and/or safety of the adenoviral-based delivery and expression system when administered to an animal as compared to administration of the same pharmaceutical composition without the corticosteroid. In some embodiments, the improvement is in a measure of one or more of pain, physical function, patient global assessment, or joint imaging of the human in need of treatment. In some embodiments, improvement is in the efficacy of treatment or progress of osteoarthritis or an osteoarthritic condition in the human joint, and is in a measure of osteoarthritis using a Western Ontario McMasters Universities Osteoarthritis (WOMAC) Index, for example WOMAC-A. In some embodiments, the improvement is in a measure of osteoarthritis using a Knee Injury and Osteoarthritis Outcome Score (KOOS). In some embodiments, the improvement is in a measure of osteoarthritis using Average daily pain (ADP) scoring system. In some embodiments, the improvement is in a measure of osteoarthritis using WOMAC, KOOS and ADP. In some embodiments, the improvement is in a measure of osteoarthritis using a physical examination of the joint of the human in need thereof, for any one or all of joint pain, joint stiffness, crepitus, redness, tenderness, Baker's Cyst and joint swelling or a combination thereof. In some embodiments, the improvement is in a measure of depression, sleep deprivation, hyperalgesia, central sensitization, and catastrophization or a combination thereof in the human subject. In some embodiments, the improvement is in a measure of osteoarthritis using radiograph imaging to determine osteophyte formation and joint space narrowing (JSN). In some embodiments, the improvement is in a measure of osteoarthritis using any one or a combination of magnetic resonance imaging (MRI), ultrasound (US), and optical coherence tomography (OCT). In some embodiments, the improvement is in a measurement of Interleukin-1 receptor antagonist (IL-1Ra) and Interleukin-1 beta (IL-1β) protein concentrations in the treated knee. In some embodiments, the improvement is in a measure of the immunological response to the helper-dependent adenoviral vector (HDAd) of the present disclosure. In some embodiments, the improvement is in a measure of osteoarthritis using blood samples of the human treated with the pharmaceutical composition or method of the present disclosure, for the presence of anti-Capsid and anti-IL-1Ra antibodies. In some embodiments, the improvement is in a measure of osteoarthritis using IL-1Ra and IL-1β protein concentrations in IA synovial fluid samples of the human treated with the pharmaceutical composition or method of the present disclosure.

### Subjects, Osteoarthritis and Osteoarthritic Condition

In some embodiments, in a method of any one of the embodiments disclosed herein the method comprises treatment of a human suffering from osteoarthritis or an osteoarthritic condition. In some embodiments, the human suffering from osteoarthritis or an osteoarthritic condition is 30-80 years of age. In some embodiments, the human suffering from osteoarthritis or an osteoarthritic condition is more than 80 years of age. In some embodiments, the human suffering from osteoarthritis or an osteoarthritic condition is-suffering from osteoarthritis of joint. In some embodiments, the human suffering from osteoarthritis or an osteoarthritic condition is suffering from osteoarthritis of shoulder, hip, ankle, knee, hand or spine. In some embodiments, the human suffering from osteoarthritis or an osteoarthritic condition has Osteoarthritis of knee (OAK). In some embodiments, the human suffering from osteoarthritis or an osteoarthritic condition has painful OA of the index knee with Kellgren-Lawrence (K-L) Grade 2, 3 or 4. In some embodiments, the human is suffering from osteoarthritis or an osteoarthritic condition caused by ageing, gender (female) related predisposition, obesity, metabolic diseases, joint injuries, repeated stress on the joint, genetic predisposition or bone deformities or a combination thereof. In some embodiments, the human is suffering from osteoarthritis or an osteoarthritic condition caused by joint injury leading to torn cartilage, dislocated joints or ligament injuries or a combination thereof. In some embodiments, the human is suffering from osteoarthritis or an osteoarthritic condition caused by anterior cruciate ligament (ACL) strains and tears. In some embodiments, the human is suffering from osteoarthritis or an osteoarthritic condition caused by meniscal strains and tears.

In some embodiments, the human suffering from osteoarthritis or an osteoarthritic condition has a Body mass index (BMI)≤40 kilograms per meters squared (kg/m²). In some embodiments, the human suffering from osteoarthritis or an osteoarthritic condition has symptoms associated with OA of the index knee for ≥12 months. In some embodiments, the human suffering from osteoarthritis or an osteoarthritic condition has Index knee pain for >15 days over the last month prior to treatment with the pharmaceutical composition or method of the present disclosure. In some embodiments, the human suffering from osteoarthritis or an osteoarthritic condition has any one or a combination of characteristics defined by American College of Rheumatology (ACR) Criteria (clinical and radiological) for OA as follows: a) Knee pain, b) at least one of (i) age>50 years; (ii) morning stiffness<30 minutes, and (iii) crepitus on knee motion, and c) osteophytes.

In some embodiments, the human suffering from osteoarthritis or an osteoarthritic condition has failed two or more types of conservative therapy for index knee osteoarthritis. In some embodiments, the human suffering from osteoarthritis or an osteoarthritic condition may have failed a structured land-based exercise program. In some embodiments, the human suffering from osteoarthritis or an osteoarthritic condition has failed prior treatment with topical non-steroidal anti-inflammatory drugs (NSAIDs). In some embodiments, the human suffering from osteoarthritis or an osteoarthritic condition has failed prior treatment with topical non-steroidal anti-inflammatory drugs (NSAIDs). In some embodiments, the human suffering from osteoarthritis or an osteoarthritic condition has failed prior treatment with nonselective NSAIDS, or COX-2 inhibitors. In some embodiments, the human suffering from osteoarthritis or an osteoarthritic condition has failed one prior type of conservative therapy and at least one prior index knee IA treatment (corticosteroid or hyaluronic acid).

In some embodiments, the human suffering from osteoarthritis or an osteoarthritic condition has a Kellgren-Lawrence (K-L) Grade 2 in the index knee based on X-ray and physical examination. In some embodiments, the human suffering from osteoarthritis or an osteoarthritic condition has a Kellgren-Lawrence (K-L) Grade 3 in the index knee based on X-ray and physical examination. In some embodiments, the human suffering from osteoarthritis or an osteoarthritic condition has a Kellgren-Lawrence (K-L) Grade 4 in the index knee based on X-ray and physical examination.

In some embodiments, the human suffering from osteoarthritis or an osteoarthritic condition has an index knee and the intended area for injection of the pharmaceutical composition of the present disclosure is free of any signs of local or joint infection. In some embodiments, the human suffering from osteoarthritis or an osteoarthritic condition has a Western Ontario and McMaster Universities Osteoarthritis Index (WOMAC) pain score in index knee between ≥4.0 and ≤9.0 (0-10 numeric rating scale [NRS] scale), including the end points. In some embodiments, the human suffering from osteoarthritis or an osteoarthritic condition is a female that is not pregnant or a female that can use one or more of methods of contraception at the time of or for at least 12 months following treatment with the pharmaceutical composition or by the method of the present disclosure.

In some embodiments, the human suffering from osteoarthritis or an osteoarthritic condition has no current or prior diagnosis of reactive arthritis, rheumatoid arthritis, psoriatic arthritis, ankylosing spondylitis, or arthritis associated with inflammatory bowel disease. In some embodiments, the human suffering from osteoarthritis or an osteoarthritic condition has no present clinical signs and symptoms of active crystal disease including gout, calcium pyrophosphate deposition disease, of the index knee. In some embodiments, the human suffering from osteoarthritis or an osteoarthritic condition has no present clinical signs and symptoms of active crystal disease within three months prior to treatment with the pharmaceutical composition or by the method of the present disclosure. In some embodiments, the human suffering from osteoarthritis or an osteoarthritic condition has no inability to undergo Magnetic Resonance Imaging (MRI) due to presence of surgical hardware or other foreign body in the index knee. In some embodiments, the human suffering from osteoarthritis or an osteoarthritic condition has no unstable index knee joint.

In some embodiments, the human suffering from osteoarthritis or an osteoarthritic condition has not received any prior treatment with intra-articular (IA) drug/biologic use in index knee, within six months of treatment with the pharmaceutical composition or by the method of the present disclosure. In some embodiments, the human suffering from osteoarthritis or an osteoarthritic condition has not received treatment with any one or a combination of corticosteroid, hyaluronic acid, platelet rich plasma, stem cells, prolotherapy and amniotic fluid injection, within six months of treatment with the pharmaceutical composition or by the method of the present disclosure. In some embodiments, the human suffering from osteoarthritis or an osteoarthritic condition has not received cold or radiofrequency nerve ablation of the index knee within 12 months of treatment with the pharmaceutical composition or by the method of the present disclosure. In some embodiments, the human suffering from osteoarthritis or an osteoarthritic condition has not undergone arthroscopic or open surgery on the index knee within 12 months of treatment with the pharmaceutical composition or by the method of the present disclosure. In some embodiments, the human suffering from osteoarthritis or an osteoarthritic condition has not planned or anticipated surgery on the index knee within 12 months of treatment with the pharmaceutical composition or by the method of the present disclosure. In some embodiments, the human suffering from osteoarthritis or an osteoarthritic condition has not suffered from loss of skin integrity over the index knee where intra-articular injection will be given.

In some embodiments, the human suffering from osteoarthritis or an osteoarthritic condition may not exhibit any laboratory evidence of infection with human immunodeficiency virus (HIV), positive test for hepatitis B surface antigen (HBsAg) or positive serology for hepatitis C virus (HCV) with positive test for hepatitis C virus ribonucleic acid (HCV RNA).

In some embodiments, the human suffering from osteoarthritis or an osteoarthritic condition may not exhibit any ECG abnormality. In some embodiments, the human suffering from osteoarthritis or an osteoarthritic condition has not received or used immunomodulators, immunosuppressive, or chemotherapeutic agents within 5 years of treatment with the pharmaceutical composition or by the method of the present disclosure. In some embodiments, the human suffering from osteoarthritis or an osteoarthritic condition has not received any prior investigational or approved gene therapy treatment does not have an active or history of malignancy within the 5 years of treatment with the pharmaceutical composition or by the method of the present disclosure, with the exception of resected basal cell carcinoma, squamous cell carcinoma of the skin, or effectively managed cervical carcinoma in situ.

In some embodiments, the human suffering from osteoarthritis or an osteoarthritic condition has not received active pharmacologic treatment for depression, including selective serotonin reuptake inhibitors (SSRIs), serotonin and norepinephrine reuptake inhibitors (SNRIs) and non-selective serotonin reuptake inhibitors (NSRIs) or tricyclics if dose/regimen has not been stable for >6 months prior to treatment with the pharmaceutical composition or by the method of the present disclosure. In some embodiments, the human suffering from osteoarthritis or an osteoarthritic condition has not done active substance abuse (drugs or alcohol) or has history of substance abuse within the 12 months prior to treatment with the pharmaceutical composition or by the method of the present disclosure. In some embodiments, the human suffering from osteoarthritis or an osteoarthritic condition has not been administered any investigational drug, biologic or device within 3 months prior to treatment with the pharmaceutical composition or by the method of the present disclosure.

In some embodiments, the human suffering from osteoarthritis or an osteoarthritic condition does not have any systemic or local bacterial or viral infection requiring intravenous (IV) antibiotics or antivirals within 4 weeks prior to treatment with the pharmaceutical composition or by the method of the present disclosure, or oral antibiotics or antivirals within 2 weeks prior to treatment with the pharmaceutical composition or by the method of the present disclosure.

In some embodiments, the human suffering from osteoarthritis or an osteoarthritic condition has bilateral knee OA, pain in the contralateral knee is not ≥4.0 (0-10 NRS scale) within 1 month prior to treatment with the pharmaceutical composition or by the method of the present disclosure. In some embodiments, the human suffering from osteoarthritis or an osteoarthritic condition has not undergone prior total or partial knee arthroplasty procedures in index knee. In some embodiments, the human suffering from osteoarthritis or an osteoarthritic condition may not have a temperature above 99.5° F. at the time of treatment with the pharmaceutical composition or by the method of the present disclosure.

In some embodiments, the human suffering from osteoarthritis or an osteoarthritic condition may not have a Prothrombin Time (PT)/International Normalized Ratio (INR)>1.5. In some embodiments, the human suffering from osteoarthritis or an osteoarthritic condition may not have an Activated Partial Thromboplastin Time (aPTT)>5 seconds above the Upper Limit of Normal (ULN).

In some embodiments, the human suffering from osteoarthritis or an osteoarthritic condition may not have Alanine aminotransferase (ALT), aspartate aminotransferase (AST)>1.5×ULN, alkaline phosphatase (ALP)>1.5×ULN, and total bilirubin outside of normal range. In some embodiments, the human suffering from osteoarthritis or an osteoarthritic condition may not have known allergy or sensitivity to acetaminophen.

In some embodiments, the human suffering from osteoarthritis or an osteoarthritic condition may not have any clinically significant acute or chronic medical conditions that would preclude the use of an IA injection or that could compromise safety of the human. In some embodiments, the human suffering from osteoarthritis or an osteoarthritic condition may not have any bleeding disorder.

In some embodiments, the human suffering from osteoarthritis or an osteoarthritic condition is administered Aspirin for cardio protection at a maximum dose of 81 milligrams (mg) per day provided the dose has been stable over the 3 months prior to treatment with the pharmaceutical composition or by the method of the present disclosure. In some embodiments, the human suffering from osteoarthritis or an osteoarthritic condition is administered medical therapy for depression, including SSRIs, SNRIs and NSRIs or tricyclics provided dose/regimen has been stable for 6 months prior to treatment with the pharmaceutical composition or by the method of the present disclosure. In some embodiments, the human suffering from osteoarthritis or an osteoarthritic condition is administered any treatment or rescue medication for adverse effects related to treatment with the pharmaceutical composition or by the method of the present disclosure.

In some embodiments, the human suffering from osteoarthritis or an osteoarthritic condition has not been administered any one of Oral NSAIDs, Topical therapies applied to the index knee cannot be any one topical NSAIDs, capsaicin, lidocaine patches, Cannabinoids, Aspirin at a dose of >325 mg per day, centrally acting pain medications, opioids, muscle relaxants, any IA injection in the index knee, cold or radiofrequency nerve ablation of the index knee, any investigational drug, device or biologic, any immunomodulator, immunosuppressive, or chemotherapeutic agents or a combination thereof. In some embodiments, the human suffering from osteoarthritis or an osteoarthritic condition has not been administered pregabalin or gabapentin. In some embodiments, the human suffering from osteoarthritis or an osteoarthritic condition human has not been administered oxycodone, hydrocodone, codeine, morphine, tramadol. In some embodiments, the human suffering from osteoarthritis or an osteoarthritic condition has not been administered cyclobenzaprine, tetrazepam, diazepam. In some embodiments, the human suffering from osteoarthritis or an osteoarthritic condition has not been administered local anesthetics, corticosteroids, hyaluronic acid, platelet rich plasma, stem cells, prolotherapy, amniotic fluid injection.

In some embodiments, in a pharmaceutical composition, method, kit or use of any one of the embodiments disclosed herein a therapeutically effective amount of the adenoviral-based biological delivery and expression system and the corticosteroid is used. As used herein, “therapeutically effective amount” or “effective amount” of the adenoviral-based biological delivery and expression system and/or corticosteroid of the disclosure has its plain and ordinary meaning as understood by one of skill in the art in view of the present disclosure, and relates generally to the amount needed to achieve a therapeutic objective. In some embodiments, this may be a complete to partial recovery from osteoarthritis or osteoarthritic condition in the joints of a human subject in need thereof. In some embodiments, this may be a partial or complete prevention of development of osteoarthritis or osteoarthritic condition in the progression of osteoarthritis in the joints of a human subject in need thereof.

As used in this disclosure and the appended claims, the singular forms “a”, “an”, and “the” include plural referents unless the context clearly dictates otherwise. Optional or optionally means that the subsequently described event or circumstance can or cannot occur, and that the description includes instances where the event or circumstance occurs and instances where it does not. For example, the phrase optionally the composition can comprise a combination means that the composition may comprise a combination of different molecules or may not include a combination such that the description includes both the combination and the absence of the combination (i.e., individual members of the combination). Ranges may be expressed herein as from about one particular value, and/or to about another particular value. When such a range is expressed, another aspect includes from the one particular value and/or to the other particular value. Similarly, when values are expressed as approximations, by use of the antecedent “about”, it will be understood that the particular value forms another aspect. It will be further understood that the endpoints of each of the ranges are significant both in relation to the other endpoint, and independently of the other endpoint.

Unless otherwise defined, all technical and scientific terms used herein have the same meaning as commonly understood by one of ordinary skill in the art to which this disclosure belongs when read in light of the disclosure. In the disclosure, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed disclosure. In the case of conflict, the present Specification, including definitions, will control. In addition, the materials, methods, and examples are illustrative only and are not intended to be limiting. Other features and advantages of the disclosure will be apparent from the following detailed description and claims. The following references disclose methods, compositions, doses, routes of administration, and sequences related to the present disclosure, and which disclosures are incorporated by reference herein, and which references are each incorporated herein by reference in their entireties: WO 2013/114199, WO 2014/115022, WO 2021/055860, and PCT/US2022/075358.

The present disclosure is further illustrated by the following non-limiting examples, the teachings and disclosure of which can be generalized and combined with the forgoing disclosure.

### EXAMPLES

### Example 1: HDAd-Mediated Gene Expression in Combination with TAC In Vitro

Methods: 116 cells or HEK293 cells were seeded in 12-well or 96-well plates in standard cell culture media. One day later, cells were infected with the gene therapy vector HDAd-LacZ (a helper-dependent adenoviral vector expressing beta-galactosidase (LacZ) under the control of the constitutive CMV promoter) at different multiplicities of infections (MOIs; virus particles per cells seeded) mixed with different amounts/concentrations of the corticosteroids triamcinolone acetonide (TAC; TriamHEXAL®, Hexal AG, Holzkirchen, Germany), betamethasone (Celestan®, MSD, Kenilworth, NJ, USA), or dexamethasone (Lipotalon®, Recordati, Milan, Italy). HDAd-LacZ was produced by GeneQuine Biotherapeutics GmbH (HDAd-LacZ originally sourced from Baylor College of Medicine, USA). In an experiment evaluating different timing or administration of the corticosteroid relative to administration of the HDAs-LacZ, cells were either pre-treated with TAC 3 hours prior to infection with HDAd-LacZ (PRE), TAC treatment and HDAd-LacZ infection was done simultaneously as described above (SIMULTANEOUS), or cells were treated with TAC 3 hours post infection with HDAd-LacZ (POST). One day later, supernatants were aspirated and cell monolayers were stained for LacZ expression using the X-Gal staining kit according to manufacturer's manual (OZ Bioscience, Marseille, France). Macroscopic and microscopic (at 4× or 10× magnification) photos of the stained monolayers were taken (blue staining indicates LacZ expression). A preliminary measure of intensity of the staining was semi-quantitatively measured using ImageJ software by converting the images to 8-bit grayscale and drawing regions of interest around the areas to be measured. The mean intensity values (on a scale of 0-255; 0 being black and 255 being white) were subtracted from 255, divided by 255 and multiplied by 100, i.e. reported values were on a scale of 0-100 (0 being white and 100 being black) where higher values indicate more intense (darker) staining.

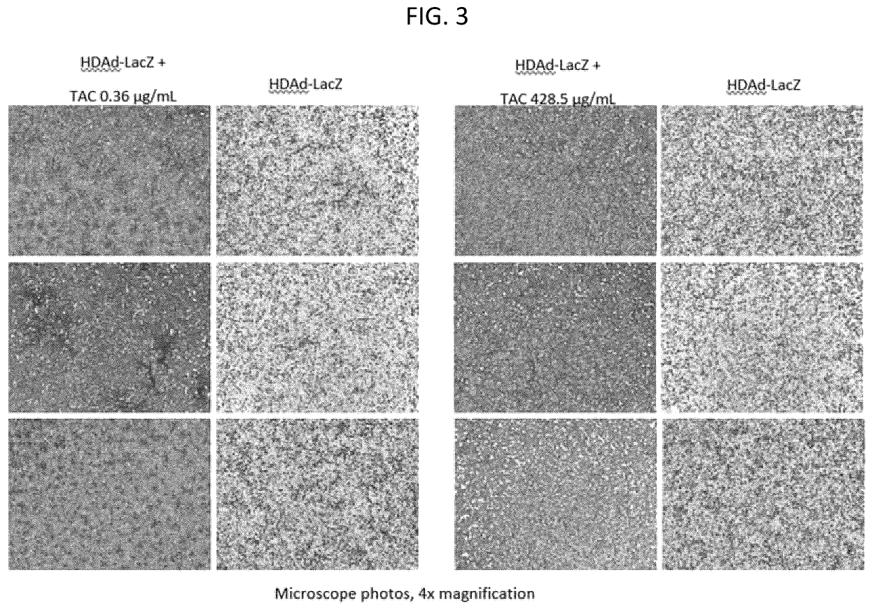

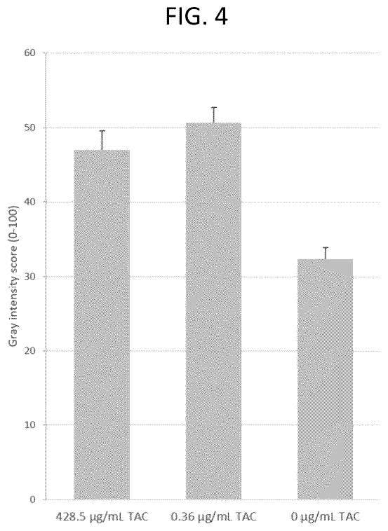

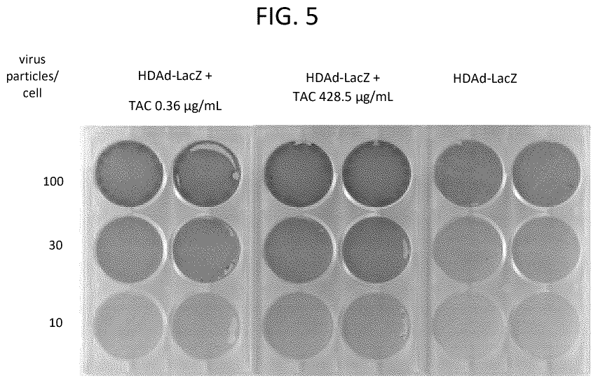

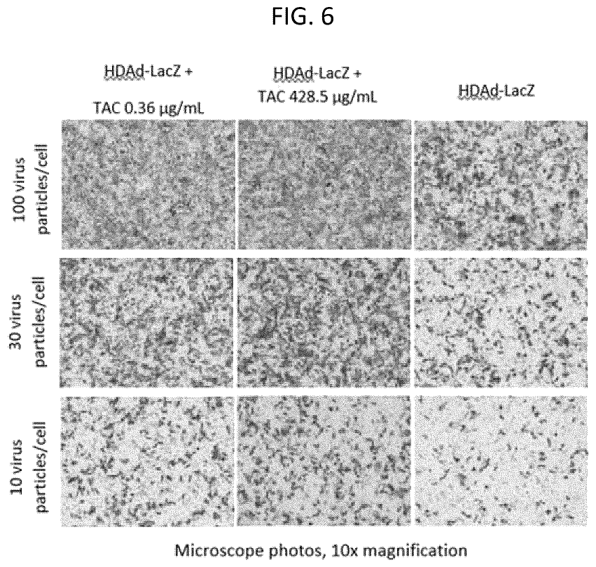

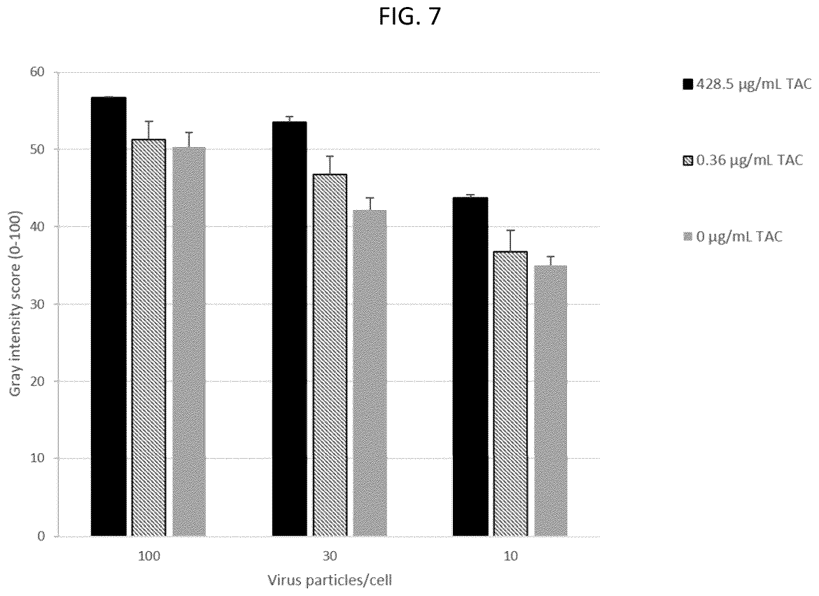

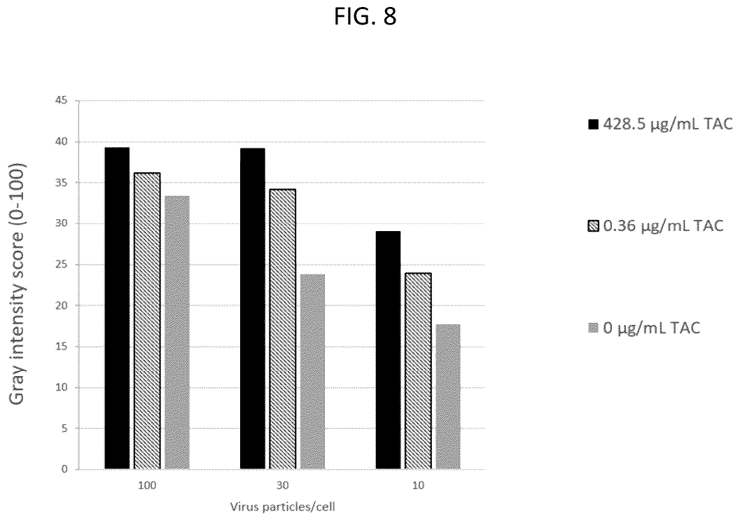

Results: HDAd-mediated LacZ expression was markedly increased by simultaneous exposure to TAC in 116 and HEK293 cells at 428.5 g/mL (resembling a dose used in humans for intraarticular treatment) and 0.36 μg/mL (a dose extrapolated from human to rat joints). FIG. 3 is microscopic photos (4× magnification) of 116 cells, and FIG. 4 represents the preliminary semi-quantitative measure of LacZ expression in the photos of FIG. 3. FIGS. 5 and 6 are macroscopic and microscopic (10× magnification) photos of HEK293 cells. FIGS. 7 and 8 represent the preliminary semi-quantitative measure of LacZ expression in the photos of FIGS. 5 and 6, respectively.

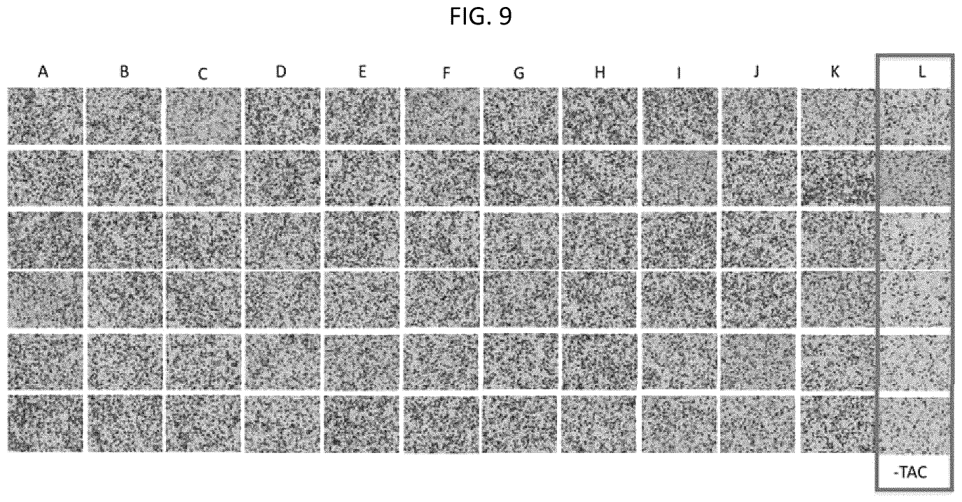

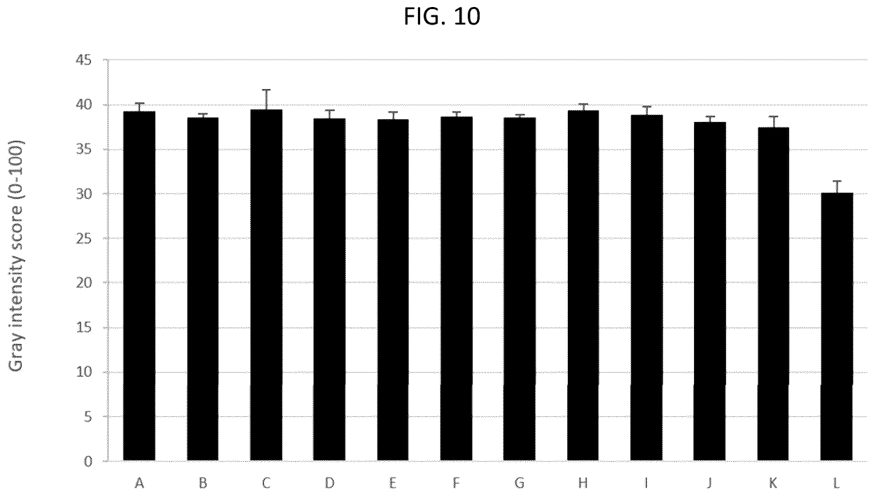

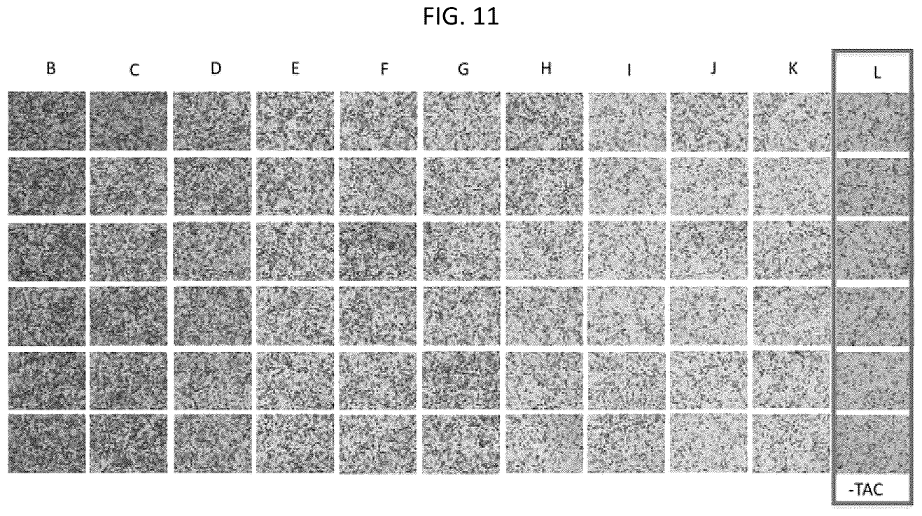

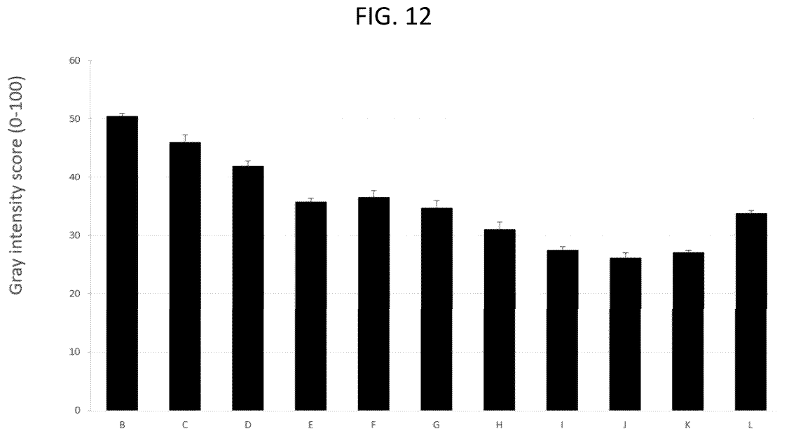

When evaluating a dose range of TAC in HEK293 cells, it appeared that enhancement of HDAd-mediated LacZ expression started at a concentration of 0.18 ng/mL. FIG. 9 is microscopic photos (10× magnification) of HEK293 cells at various concentrations of TAC (groups A-K: 2.88 μg/mL-0.0028125 μg/mL; group L: 0 ng/ml of TAC), and FIG. 10 represents the preliminary semi-quantitative measure of LacZ expression in the photos of FIG. 9. FIG. 11 is microscopic photos (10× magnification) of HEK293 cells at various concentrations of TAC ((groups B-K: 2.8125 ng/ml-0.005493164 ng/mL; group L: 0 ng/ml of TAC), and FIG. 12 represents the preliminary semi-quantitative measure of LacZ expression in the photos of FIG. 11.

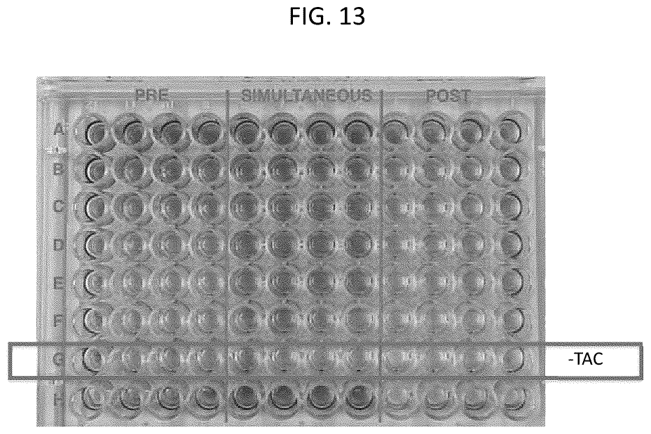

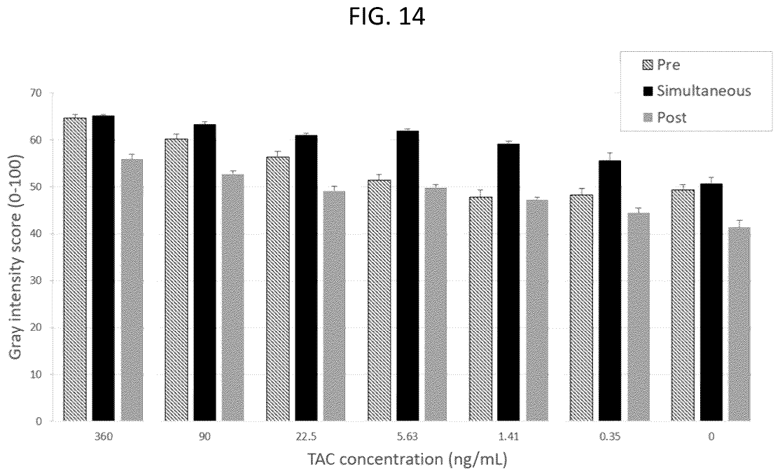

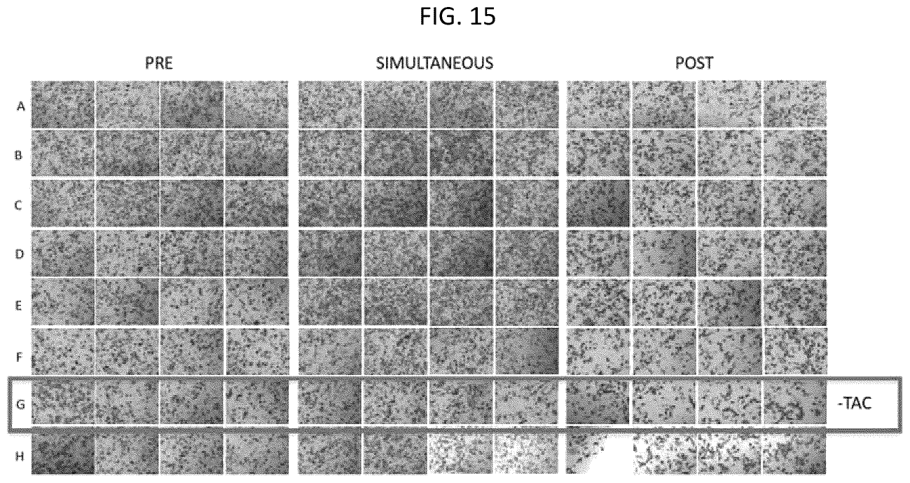

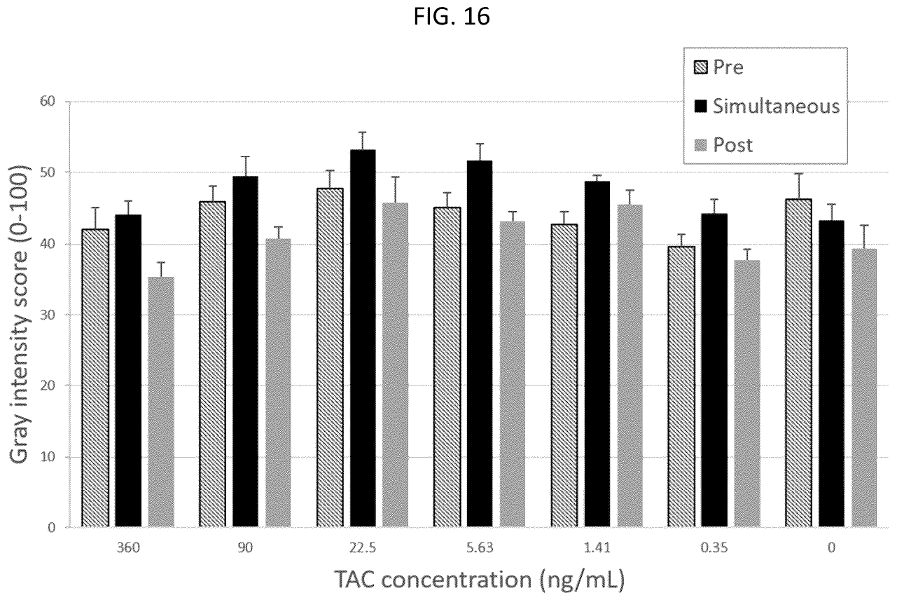

The gene expression enhancement induced by TAC was most pronounced when HEK293 cells were simultaneously treated with TAC and HDAd-LacZ compared with a 3 hour pre-treatment with TAC and treatment with TAC post HDAd-LacZ infection. FIG. 13 is a macroscopic photo, and FIG. 14 represents the preliminary semi-quantitative measure of LacZ expression from the photo of FIG. 13. FIG. 15 is microscopic (10× magnification) of the plate shown in FIG. 13, and FIG. 16 represents the preliminary semi-quantitative measure of LacZ expression from the microscopic photos of FIG. 15. The preliminary assessment indicates that simultaneous administration improves staining as compared to pretreatment 3 hours prior to infection, which in turn improves staining as compared to posttreatment 3 hours after infection.

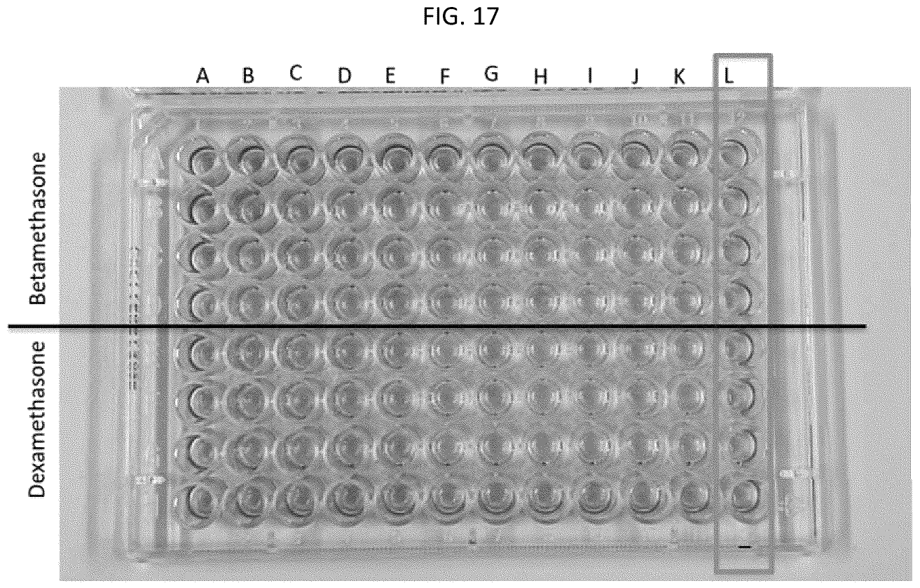

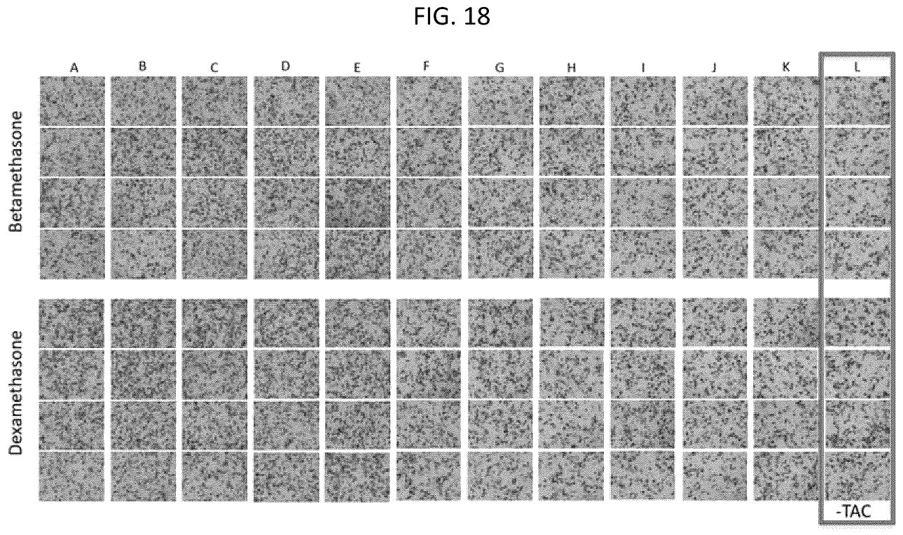

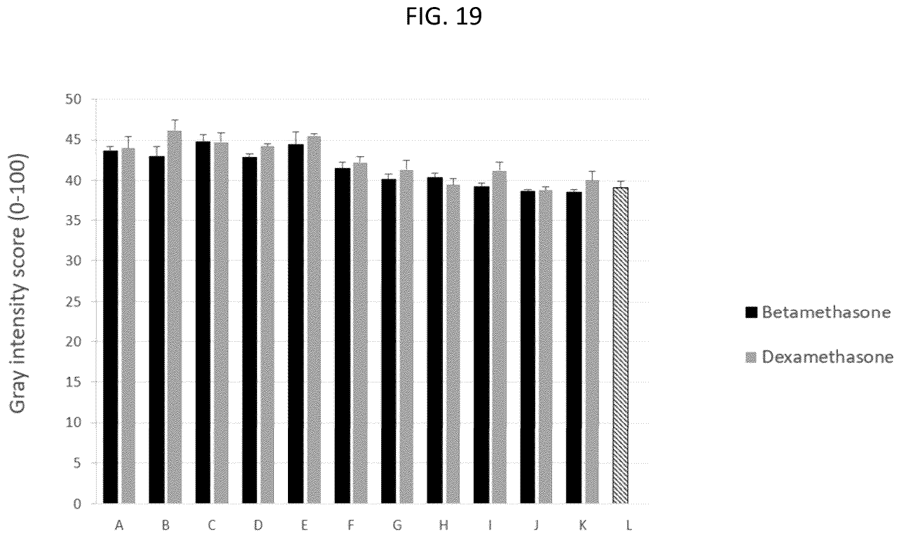

Two additional corticosteroids were tested. Betamethasone and dexamethasone both increased HDAd-mediated LacZ expression. FIGS. 17 and 18 are macroscopic and microscopic photos, respectively, and FIG. 19 represents the preliminary semi-quantitative measure of LacZ expression from the microscopic photos of FIG. 18). For both betamethasone and dexamethasone, the enhancement appeared to start at a concentration of 3.9 ng/mL.

### Example 2: HDAd-Mediated Gene Expression in Combination with TAC In Vivo in Rat Joints

In vivo studies were carried out at Bolder BioPATH (now Inotiv), Boulder, CO, USA. The study design and animal usage was approved by Bolder BioPATH's Institutional Animal Care and Use Committee (IACUC).

Rats obtained from Envigo (Indianapolis, IN) were housed in polycarbonate cages, 4 animals per cage. After 4 days acclimatization, on study day 0, animals (mean weight of 310 g) were randomized into groups by bodyweight, 6 animals per group. Following randomization, test article administration was performed by intraarticular injection of 50 μL/joint. In respective groups, animals were treated with: 5E7 VP HDAd-LacZ, 5E8 VP HDAd-LacZ, 5E9 VP HDAd-LacZ, 5E10 VP HDAd-LacZ, 5E7 VP HDAd-LacZ+0.06 mg TAC, 5E8 VP HDAd-LacZ+0.06 mg TAC, 5E9 VP HDAd-LacZ+0.06 mg TAC, 5E10 VP HDAd-LacZ+0.06 mg TAC or vehicle.

For the groups co-treated with HDAd-LacZ and TAC, TAC suspension was prepared by diluting the commercially available injectable suspension (Kenalog 10, Bristol-Myers Squibb) with isotonic saline solution and added to the HDAd-LacZ preparation (i.e. the injection volume of 50 μL of the test article+TAC mixture contained 0.06 mg TAC).

At necropsy on study day 4, the rats were anesthetized with isoflurane. Following knee lavage, knees were harvested, fixed in chilled 2% paraformaldehyde (PFA) for 1 hour, then washed 3 times with PBS. The joints were incubated at 37° C. for eight hours (or until blue color fully developed) in a 1.2 mg/mL X-gal solution (pH 7.4). The knee joints were photographed, dissected further as needed, and fixed for an additional 48 hours in 10% neutral buffered formalin (NBF). Preserved knee joints were decalcified in formic acid, embedded in paraffin, sectioned, and stained with Nuclear Fast Red (NFR) stain. Three sections were cut from each joint.

The tissues were evaluated by the investigator microscopically for inflammation and X-gal staining in a blinded manner. The approximate lengths of the medial and lateral synovia were measured using an ocular micrometer. The lengths of the X-gal-stained areas were measured, and the length of staining relative to the total length of the area (%) was determined. The width of X-gal labeling that spread beyond the synovial lining into the subsynovial periarticular tissue was estimated to approximate the total area of staining (%). If the staining was restricted to the synovial lining and immediate subsynovial area, it was given a width measure of 0.5 units. Any width measures greater than 0.5 indicated that, in addition to possible synoviocyte staining, the periarticular connective tissue cells (mainly fibroblasts) had staining (extending out from the lining).

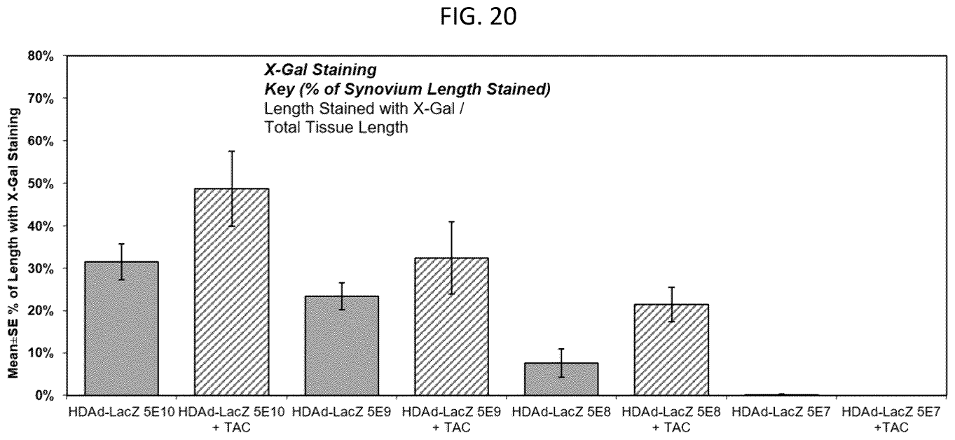

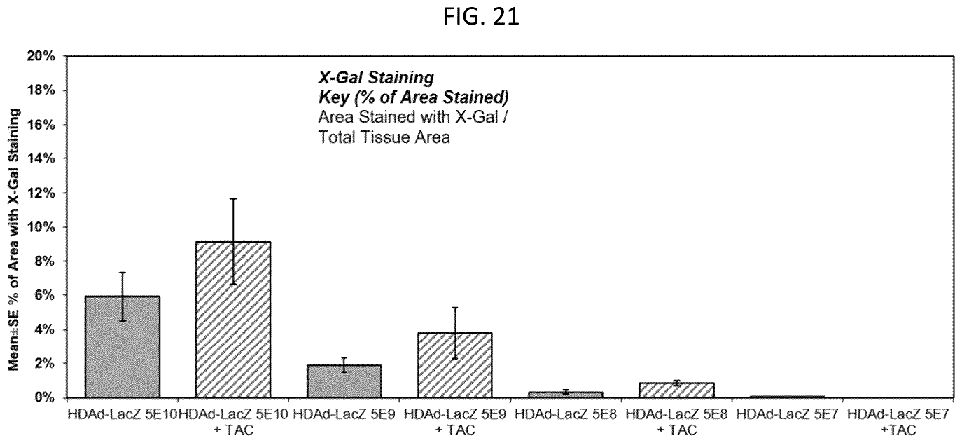

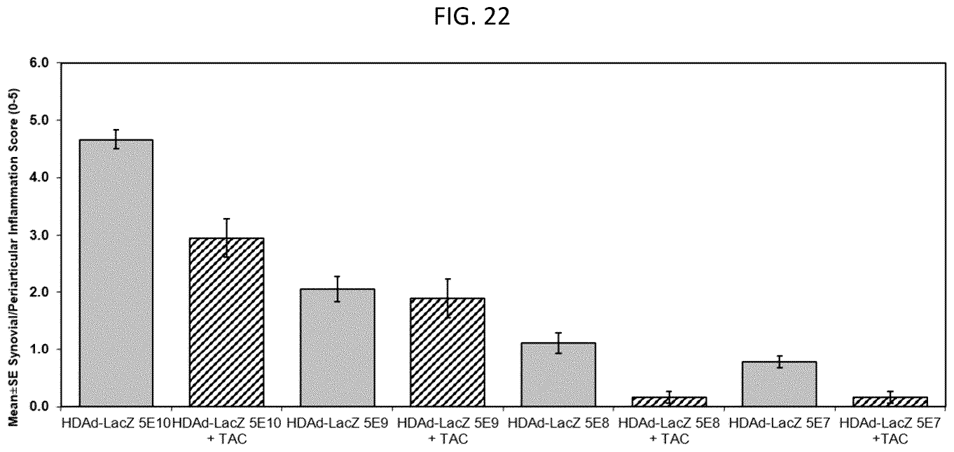

Results of the X-gal staining evaluation show that intraarticular co-administration of HDAd-LacZ and TAC leads to increased transduction and higher LacZ expression in synovium tissue, which is the primary target tissue for HDAd-mediated intraarticular gene therapy (FIGS. 20 and 21). FIG. 22 shows that coadministration of TAC reduced HDAd-LacZ mediated inflammation. Interestingly, results of the X-gal staining length (FIG. 20) indicates that co-injection with TAC may lead to increase of the transduction and expression to the levels similar to those with 10× higher dose levels (31.43% in 5E10 VP group vs. 32.40% in 5E9+TAC group; 23.43% in 5E9 VP group vs. 21.49% in 5E8+TAC group). This finding indicates that the dose for clinical use can be meaningfully reduced by co-administration of HDAd vectors with TAC or other corticosteroids. This can reduce the production needs, costs per dose, total virus burden to the patient and drug-related adverse events, if any.

### Example 3: Improved Efficacy and Safety of PCRX201 (Aka FX201) Combined with Corticosteroid Treatment for Knee Osteoarthritis

Methods: This Phase 1, open-label, proof-of-concept, single ascending dose study enrolled adults aged 30 to 80 years with moderate to severe knee osteoarthritis (OA) (NCT04119687). Additional inclusion criteria Western Ontario and McMaster Universities Osteoarthritis Index pain score (WOMAC-A)>4.0 and ≤9.0, Kellgren-Lawrence (K-L) grades 2 through 4, and prior failure of >2 other treatments for OA. The first half of patients received ultrasound-guided intra-articular injection of 5 ml of PCRX201 to the knee joint at ascending doses of 2.8E9 genome copies (GC)/mL (1.4E10 GC/knee) (low-dose), 2.8E10 GC/mL (1.4E11/knee) (mid-dose), and 2.8E11 GC/mL (1.4E12/knee) (high-dose). The second half of patients received a 1 ml intra-articular injection of methylprednisolone (40 mg/ml) just before PCRX201 administration at the above doses. The methylprednisolone was administered first, followed by the PCRX201 administered through the same needle without removing it from the subject's knee between administrations. The primary endpoint was safety, which included adverse event (AE) monitoring, repeated index knee assessments, laboratory evaluations, and biodistribution samples. Efficacy was assessed using the change from baseline in pain (WOMAC-A). The current data represent preliminary results based on data available, with no imputations of missing values, through up to 6 months after treatment administration to the last patient.

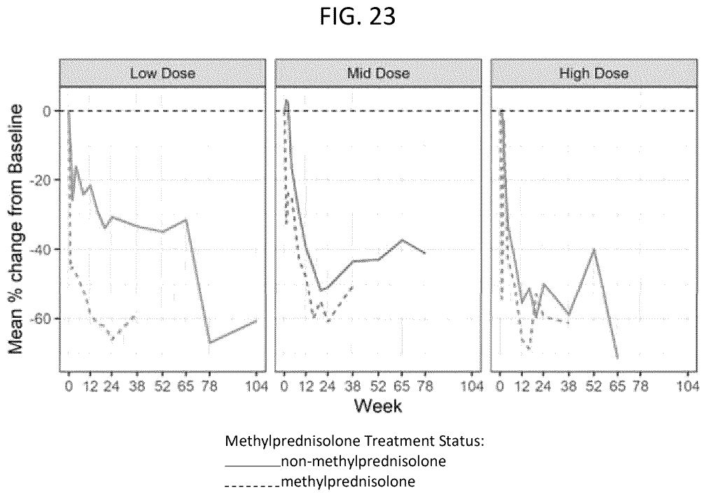

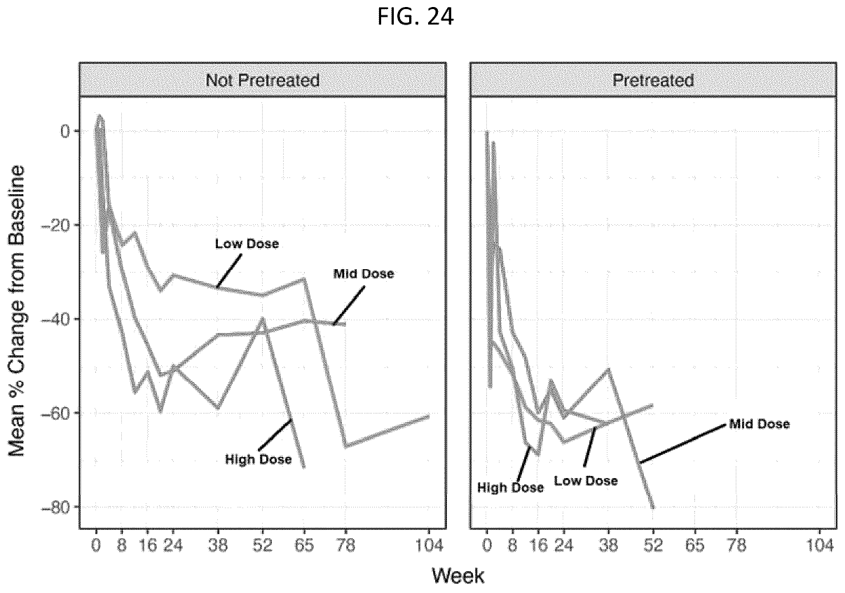

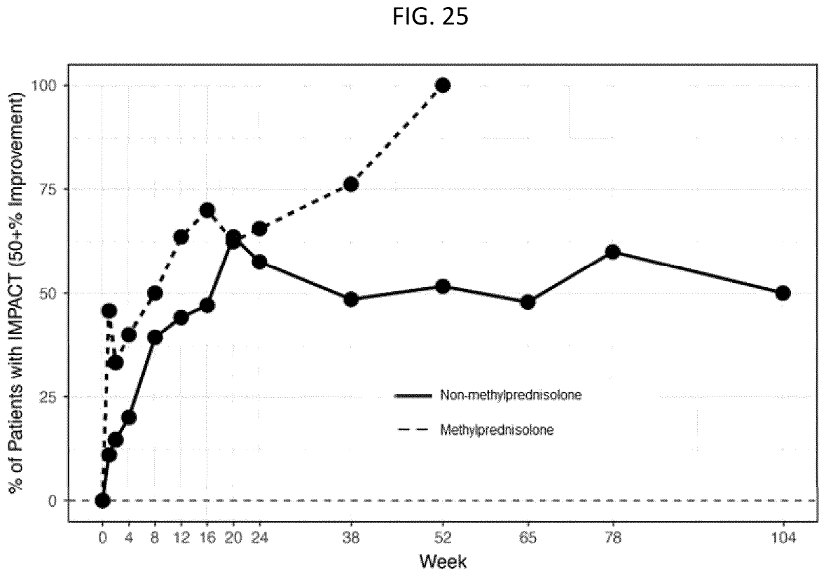

Results: Seventy-two patients were enrolled and treated, with 36 patients in each cohort (non-methylprednisolone and methylprednisolone). The first patient was dosed in March 2020 and the last patient was dosed in December 2021. As of May 2022, 33% of the patients have discontinued the study. Low-, mid-, and high-dose PCRX201 were given to 12, 16, and 8 patients in the non-methylprednisolone cohort, respectively; in the methylprednisolone cohort, low-, mid-, and high-dose PCRX201 were given to 13, 15, and 8 patients, respectively. Index knee effusions considered related to PCRX201 were dose dependent (50%, 56%, and 100% by ascending dose in the non-methylprednisolone cohort and 31%, 27%, 63% by ascending dose in the methylprednisolone cohort). There were 5 (14%) grade 3 knee events in the non-methylprednisolone cohort and 1 (7%) in the methylprednisolone cohort. Most of the index knee AEs were treated conservatively with rest, ice, acetaminophen, and aspiration of synovial fluid. In the high-dose groups, 4 patients experienced self-limited chills without fever (2 in each cohort), 2 had concurrent headache (1 in each cohort). One patient (high-dose) in the methylprednisolone cohort had flu-like symptoms and 1 patient (mid-dose) in the non-methylprednisolone cohort had self-limited fever at the onset of a knee AE. One patient (mid-dose) in the methylprednisolone cohort experienced severe pain during PCRX201 administration resulting in early termination of the injection. There were no other PCRX201-related AEs. Baseline adenovirus status was not predictive of index knee AEs and there was no clinical evidence of biodistribution. Improvement in pain as assessed by percent change from baseline in WOMAC-A scores was noted for each dose group in each cohort (FIGS. 23 and 24). FIG. 23 compares methylprednisolone to non-methylprednisolone treated patients for each of the three doses of PCRX201 tested. FIG. 24 includes additional data, but is grouped by methylprednisolone treatment status, pretreated referring to administration of methylprednisolone immediately prior to PCRX201 as discussed above. Of note is the overall greater response and speed of onset for the methylprednisolone cohort at at least the low and mid dose of PCRX201. FIG. 25 displays the incidence of WOMAC-A 50% responders over time for the methylprednisolone treated and non-treated groups. Of note is the 100% rate of 50% responders for the methylprednisolone cohort at week 52 compared to only 50% rate for the non-methylprednisolone cohort.

Conclusions: PCRX201 was generally well tolerated with decreased incidence and severity of index knee events in the methylprednisolone cohort compared with the non-methylprednisolone cohort. Preliminary efficacy results were promising and suggested substantial improvement in pain across all doses studied in this phase 1 open-label study, with faster onset and greater overall response rates observed for the methylprednisolone cohort.

1. A pharmaceutical composition comprising a corticosteroid and an adenoviral-based delivery and expression system comprising a nucleic acid encoding a protein for expression by the adenoviral-based delivery and expression system.

2. (canceled)

3. The pharmaceutical composition of claim 1, wherein the corticosteroid is selected from the group consisting of:
beclomethasone, betamethasone, budesonide, cortisone, deflazacort, dexamethasone, hydrocortisone, methylprednisolone, prednisolone, prednisone, triamcinolone, fludrocortisone, deoxycorticosterone, aldosterone, ciclesonide, fluticasone, flunisolide, mometasone, and mixtures thereof;
beclomethasone, betamethasone, budesonide, cortisone, deflazacort, dexamethasone, hydrocortisone, methylprednisolone, prednisolone, prednisone, triamcinolone, fludrocortisone, deoxycorticosterone, aldosterone, and mixtures thereof;
beclomethasone, betamethasone, budesonide, cortisone, dexamethasone, hydrocortisone, methylprednisolone, prednisolone, prednisone, triamcinolone, and mixtures thereof;
beclomethasone, betamethasone, budesonide, cortisone, dexamethasone, hydrocortisone, methylprednisolone, prednisolone, triamcinolone, and mixtures thereof;
betamethasone, dexamethasone, triamcinolone, methylprednisolone, and mixtures thereof;
beclomethasone, budesonide, ciclesonide, fluticasone, flunisolide, mometasone, and mixtures thereof; and/or
fludrocortisone, deoxycorticosterone, aldosterone, and mixtures thereof.

4-5. (canceled)

6. The pharmaceutical composition of claim 1, wherein the adenoviral-based delivery and expression system is a recombinant adenoviral vector or a helper-dependent adenoviral vector, optionally an Ad5 serotype.

7-33. (canceled)

34. The pharmaceutical composition of claim 1, wherein the concentration of the corticosteroid is sufficient to improve one or more of transduction, expression, efficacy and/or safety of the adenoviral-based delivery and expression system when administered to an animal as compared to administration of the same pharmaceutical composition without the corticosteroid.

35-43. (canceled)

44. The pharmaceutical composition of claim 1, wherein the corticosteroid is in a separate dosage form from the adenoviral-based biological delivery and expression system such that the corticosteroid can be administered before or after the adenoviral-based biological delivery and expression system.

45. (canceled)

46. The pharmaceutical composition of claim 1, wherein a single dosage form comprising the corticosteroid and the adenoviral-based biological delivery and expression system is prepared by combining a composition comprising the corticosteroid and a composition comprising the adenoviral-based biological delivery and expression system.

47. A kit comprising the pharmaceutical composition of claim 44, wherein the kit comprises a first container comprising the corticosteroid in a first dosage form, and a second container comprising the adenoviral-based biological delivery and expression system in a second dosage form.

48. A kit for use in preparing the pharmaceutical composition of claim 46, wherein the kit comprises the composition comprising the corticosteroid in a first container and the composition comprising the adenoviral-based biological delivery and expression system in a second container.

49. A method comprising administering to an animal the pharmaceutical composition of claim 1, wherein the administration of the pharmaceutical composition infects cells of the animal with the adenoviral-based delivery and expression system, resulting in expression of the protein encoded by the nucleic acid of adenoviral-based delivery and expression system.

50-51. (canceled)

52. The method of claim 49, wherein the concentration of the corticosteroid is sufficient to improve one or more of transduction, expression, efficacy and/or safety of the adenoviral-based delivery and expression system when administered to the animal as compared to administration of the same pharmaceutical composition without the corticosteroid.

53. The method of claim 52, wherein the improvement comprises an improvement in the quantity or duration of a measure of transduction or expression.

54. (canceled)

55. The method of claim 52, wherein the improvement in safety comprises a reduction in the number, severity or duration of an adverse event, optionally wherein the adverse event is selected from pain, swelling, effusion, inflammation, headache, fever, chills, and flu-like symptoms.

56. The method of claim 52, wherein the improvement in efficacy comprises an improvement in the onset, amount or duration of a therapeutic effect of the adenoviral-based delivery and expression system.

57. The method of claim 56, wherein the therapeutic effect comprises a reduction in a measure of a symptom or marker of a disease or disorder; optionally wherein the symptom is pain in a joint of the animal, optionally as measured using the Western Ontario and McMaster Universities Osteoarthritis Index pain score (WOMAC-A).

58-60. (canceled)

61. The method of claim 49, wherein the corticosteroid is administered to the animal a period of time that is, or is not more than 0.00, 0.01, 0.05, 0.1, 0.2, 0.3, 0.4, 0.5, 1, 1.5, 2, 2.5, 3, 4, or 5 hours prior to the administration of the adenoviral-based delivery and expression system.

62-67. (canceled)

68. The method of claim 49, wherein the method comprises administration of the corticosteroid and adenoviral-based delivery and expression system of the pharmaceutical composition locally to the same location of the animal.

69-71. (canceled)

72. The method of claim 68, wherein the injection is an intra-articular injection, optionally wherein the joint is the knee.

73. The method of claim 68, wherein the method comprises injection into an intervertebral disc; optionally wherein the injection is intradiscal injection, optionally into the nucleus pulposus (NP) region of the intervertebral disc.

74-76. (canceled)

77. The method of claim 49, wherein the method comprises treating a disease or condition, wherein the disease or condition is:
camptodactyly-arthropathy-coxa vara-pericarditis (CACP) syndrome;
a musculoskeletal disease or condition;
a joint disease or condition;
a lung disease or condition;
an eye disease or condition;
an ear disease or condition;
arthropathies, arthritis, or arthritis-related conditions;
osteoarthritis, rheumatoid arthritis, gout and pseudo-gout, septic arthritis, psoriatic arthritis, ankylosing spondylitis, juvenile idiopathic arthritis, Still's disease, Reiter's syndrome, or tendinopathies including tendonitis, tendinosis, and tenosynovitis;
synovial disorders including synovitis;
a bursa disorder, including bursitis; or
equine musculoskeletal disorders including bone spavin, navicular syndrome, and osselet.

78-88. (canceled)

89. The method of claim 49, wherein the adenoviral-based delivery and expression system is a recombinant adenoviral vector or a helper-dependent adenoviral vector, and the amount of vector administered to the animal is, is at least, or is not more than, 1.4×10⁵, 1.4×10⁶, 2.8×10⁶, 7.0×10⁶, 1.4×10⁷, 2.8×10⁷, 7.0×10⁷, 1.4×10⁸, 2.8×10⁸, 7.0×10⁸, 1.4×10⁹, 2.8×10⁹, 7.0×10⁹, 1.4×10¹⁰, 2.8×10¹⁰, 7.0×10¹⁰, 1.4×10¹¹, 2.8×10¹¹, 7.0×10¹¹, 1.4×10¹², 2.8×10¹², 7.0×10¹², 1.4×10¹³, 2.8×10¹³, 7.0×10¹³ or a range defined by any two of the preceding values, optionally 1.4×10⁵ to 7.0×10¹³, 1.4×10⁵ to 7.0×10¹², 1.4×10⁵ to 7.0×10¹¹, 1.4×10⁵ to 7.0×10¹⁰, 1.4×10⁵ to 7.0×10⁹, 1.4×10⁵ to 7.0×10⁸, 1.4×10⁹ to 7.0×10¹⁰, optionally 1.4×10⁵, 1.4×10⁷, 5.6×10⁹, 7.0×10⁹, 1.4×10¹⁰, 1.4×10¹¹, or 1.4×10¹², optionally 5.6×10⁹, 7.0×10⁹, 1.4×10¹⁰, 1.4×10¹¹, or 1.4×10¹², genome copies (GC) of recombinant adenoviral vector or a helper-dependent adenoviral vector.

90-93. (canceled)

94. The method of claim 49, wherein the amount of corticosteroid administered to the animal is, is at least, is not more than, 0.001, 0.01, 0.1, 1.0, 5.0, 10, 15, 20, 25, 30, 40, 50, 60, 70, 80, 90, 100, 250, 500, 750, 1000, 2500, or 5000, or a range defined by any two of the preceding values, optionally 0.001-5000, 0.001-0.1, 0.001-10, 0.1-100, 1.0-100, 1.0-50, 30-300, 25-60, 30-50, 100-1000, 500-5000 mg, optionally 40 mg.

95. The method of claim 49, wherein the method comprises:
identifying a human subject as suffering from osteoarthritis of the knee,
administering the pharmaceutical composition to the human subject by intra-articular injection into an osteoarthritis-affected knee joint,
wherein the corticosteroid is in a separate dosage form from the adenoviral-based biological delivery and expression system,
wherein administration of the corticosteroid comprises intra-articular injection of 1 ml of a dosage form comprising 40 mg/ml of the corticosteroid, wherein the corticosteroid is methylprednisolone,
wherein administration of the adenoviral-based biological delivery and expression system comprises intra-articular injection of 5 ml of a dosage form comprising per ml of the adenoviral-based biological delivery and expression system,
wherein the amount of adenoviral-based biological delivery and expression system delivered to the knee is 1.4×10¹⁰, 1.4×10¹¹, or 1.4×10¹², genome copies (GC) a helper-dependent adenoviral vector,
wherein the administration of the corticosteroid and the adenoviral-based delivery and expression system is through the same needle and the needle is maintained in the same site during administration of both the corticosteroid and the adenoviral-based delivery and expression system, optionally wherein the needle is flushed with a fluid between corticosteroid and the adenoviral-based delivery and expression system,
wherein the corticosteroid is administered not more than 0-15 minutes, optionally 0-5 minutes, before administration of the adenoviral-based delivery and expression system,
wherein the adenoviral-based delivery and expression system comprises the helper-dependent adenoviral vector, and
wherein the helper-dependent adenoviral vector comprises a nucleic acid sequence encoding human IL-1Ra protein, left and right inverted terminal repeats, an adenoviral packaging signal and stuffer nucleic acid sequences that are non-viral and non-coding, wherein the expression of the nucleic acid sequence encoding human IL-1Ra protein is regulated by a NF-κB inducible promoter, which is located upstream of the reading frame of the nucleic acid sequence encoding the human IL-1Ra protein.

96-109. (canceled)

110. The method of claim 49, wherein the corticosteroid is selected from the group consisting of:
beclomethasone, betamethasone, budesonide, cortisone, deflazacort, dexamethasone, hydrocortisone, methylprednisolone, prednisolone, prednisone, triamcinolone, fludrocortisone, deoxycorticosterone, aldosterone, ciclesonide, fluticasone, flunisolide, mometasone, and mixtures thereof;
beclomethasone, betamethasone, budesonide, cortisone, deflazacort, dexamethasone, hydrocortisone, methylprednisolone, prednisolone, prednisone, triamcinolone, fludrocortisone, deoxycorticosterone, aldosterone, and mixtures thereof;
beclomethasone, betamethasone, budesonide, cortisone, dexamethasone, hydrocortisone, methylprednisolone, prednisolone, prednisone, triamcinolone, and mixtures thereof;
beclomethasone, betamethasone, budesonide, cortisone, dexamethasone, hydrocortisone, methylprednisolone, prednisolone, triamcinolone, and mixtures thereof;
betamethasone, dexamethasone, triamcinolone, methylprednisolone, and mixtures thereof;
beclomethasone, budesonide, ciclesonide, fluticasone, flunisolide, mometasone, and mixtures thereof; and/or
fludrocortisone, deoxycorticosterone, aldosterone, and mixtures thereof.


SEQUENCE LISTING (19 sequences)

SEQ ID NO: 1 - DNA, 535 bp, synthetic construct
  misc_feature 1..535: note = murine Il-1Ra
  source 1..535: mol_type = other DNA
atggaaatctgctggggaccctacagtcacctaatctctctccttctcatccttctgtttcattcagaggcagcctgccgcccttctgggaaaagaccctgcaagatgcaagccttcagaatctgggatactaaccagaagaccttttacctgagaaacaaccagctcattgctgggtacttacaaggaccaaatatcaaactagaagaaaagatagacatggtgcctattgaccttcatagtgtgttcttgggcatccacgggggcaagctgtgcctgtcttgtgccaagtctggagatgatatcaagctccagctggaggaagttaacatcactgatctgagcaagaacaaagaagaagacaagcgctttaccttcatccgctctgagaaaggccccaccaccagctttgagtcagctgcctgtccaggatggttcctctgcacaacactagaggctgaccgtcctgtgagcctcaccaacacaccggaagagccccttatagtcacgaagttctacttccaggaagaccaat

SEQ ID NO: 2 - DNA, 29612 bp, synthetic construct
  misc_feature 1..29612: note = helper-dependent adenoviral vector-murine Il-1Ra gene
  source 1..29612: mol_type = other DNA
aaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaactggacatttgttctaatctttgaagagtatgaaaaattcctattttaaaggtaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcatttcaattgtgtgacacaggcagactgtggatcaaaagtggtttctggtgcgacttactctcttgagtatacctgcagtcccctttcttaagtgtgttaaaaaaaaagggggatttcttcaattcgccaatactctagctctccatgtgctttctaggaaacaagtgttaacccaccttatttgtcaaacctagctccaaaggacttttgactccccacaaaccgatgtagctcaagagagggtatctgtcaccagtatgtatagtgaaaaaagtatcccaagtcccaacagcaattcctaaaaggagtttatttaaaaaaccacacacacctgtaaaataagtatatatcctccaaggtgactagttttaaaaaaacagtattggctttgatgtaaagtactagtgaatatgttagaaaaatctcactgtaaccaagtgaaatgaaagcaagtatggtttgcagagattcaaagaaaatataagaaaacctactgttgccactaaaaagaatcatatattaaatatactcacacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtatcacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaattttctggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacacactaatattaaaagtgtccaataacatttaaaactatactcatacgttaaaatataaatgtatatatgtacttttgcatatagtatacatgcataggccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatgaggaataaacaccctttccaaatcctcagcataatgattaggtatgcaaaataaatcaaggtcataacctggttcatcatcactaatctgaaaaagaaatatagctgtttcaatgagagcattacaggatacaaacatttgattggattaagatgttaaaaaataaccttagtctatcagagaaatttaggtgtaagatgatattagtaactgttaactttgtaggtatgataatgaattatgtaagaaaacaacaggccgggcgggttggttcacacgtgtaatcccagcactttgggaggctgaggcaggcagactgcctgagctcaggagttcgagaccagcctgggcaacacggtgaaatcccgtctctactaaaaatacaaaaaaattagccgggtgtggtgacacatgcctgtagtcccagctacttgggaggctgaggcaggagaatcacttgaacctgggaggtgaaggttgcagtgagccaagatggcaccacttcactccagcctgggaaacagagcaagactctgtctctgagctgagatggcaccacttcactccagcctgggaaacagagcaagactctgtctcaaaaaaaacaaaacacacaaacaaaaaaacaggctgggcgcggtggctcacgcctgtaatcccagcactttgggaggccgaggcgggtggatcacctgaggtcaggagttccagaccagccttgtcaacatggtgaaacctccccccgccgtctctactaaaaatacaaaaattagccaggcgtggtggcaggagcctgtaatcccagctacttgggaggctgaggcaggagaatcgcttgtacccagaaggcagaggttgcactgagctgagatggcaccattgcactccagcctgggggacaagagcgagatttcgtctttaaaaaacaaaaacaaaacaaaaaaccatgtaactatatgtcttagtcatcttagtcaagaatgtagaagtaaagtgataagatatggaatttcctttaggtcacaaagagaaaaagaaaaattttaaagagctaagacaaacgcagcaaaatctttatatttaataatattctaaacatgggtgatgaacatacgggtattcattatactattctctccacttttgagtatgtttgaaaatttagtaaaacaagttttaacacactgtagtctaacaagataaaatatcacactgaacaggaaaaactggcatggtgtggtggctcacacttgtaatcccagtgctttgggaggctgagacaggagagttgcttgaggccaggagttcaagaccgacatggggaatgtagcaagaccccgtccctacaaaaaactttgtaaaaatttgccaggtatggtggtgcatacctgtagtcccagctactcgggaggcggaggcagaaggaatcacttgagcccaggagtttgaggctgcagtgagctacgatcataccacagcactccagcgtggacaacagagtaagaccctatctcaaaaacaaaacaaaacaaaacaaacaaaaaaaaccacaagaaaaactgctggctgatgcagcggctcatgcctgtaatcccagtattttgggaggcccaggtgggcgtatcacctgaggtcaggagttagagaccagcctggccaacatggtgaaaccccatctctactaaaaatacaaaattagccaggcatgtggcacgcgcctgtagtcccagttactgggaggctgaagcaggaggatcacctgagcccgggaggtggaggttgcagtgagccgagatcacaccactgcactccagcctgggtgacacagcaataccctacctcaaaataaaaaagaaaaagaaaagaaaagttgctgtccccgctaccccaatcccaaatccaaacagcctctctcatctcacagtaagggggaaaaatcacccaaaaaagctaagtgatcttttgaaaacccaaactcttagaagtctaagattattatagtcaactcatgaagtgtcatcataaaagatactctaatattatttaagtagaaccacatattggttgtcttggtatgtctagcccctggcatacaaaatatttaataacactgatatggtacctgtgatgtgaaaatgtactatgagtacagctttataaatactatatatgtacctatatacagaaaaaaatacaacaaaatcataaaagcacttatctttgaaagaggagttacagcaattttatttagttctttattgctttgctatatattctaaatttttttcaatgaatatatatcacttttaaaaaaattcaatggtctttcttataaattatctttggcagcatgcgtttttatatatacatataaaatgtatgggaaatttttaaaggatacattaaattaaagcaaaatatacaaacaaaaaatcagaatacaaaaagataaaaagattgggaagggagggagggagtaaggaggaagggtgggtgggtatagagaaatataccaaataatggtaagaagtggggtcttgacactttctacactttttttaaataaaaaaaatttttttctctctcttttttttttttagagacgaagtctcgctatgttgcccaggctggtcttgaactcctgggatcaagagatcctcctgcctcagcctcccaaggtgcttggattacaggtgtgagccaccacgcctggtcactttctacactttaatatatatattttttcattttcaatgtcatttttattagttaatttataatacccattcaccattatattcaaagtctatttgaagaaataaaccagaaagaatgaaatactctagctcacatgctattcaatactaaattacctttcaaatcacattcaagaagctgatgatttaagctttggcggtttccaataaatattggtcaaaccataattaaatctcaatatatcagttagtacctattgagcatctccttttacaacctaagcattgtattaggtgcttaaatacaagcagcttgacttttaatacatttaaaaatacatatttaagacttaaaatcttatttatggaattcagttatattttgaggtttccagtgctgagaaatttgaggtttgtgctgtctttcagtccccaaagctcagttctgagttctcagactttggtggaacttcatgtattgtcaggttggcccgtaatacctgtgggacaacttcagcccctgtgcacatggccaggaggctggttgcaaacattttcaggtaggtggaccaggacatgcccctggtcatggccaggtggaggcatagtgctatacagcaggcagaagtcaatattgatttgtttttaaagaaacatgtactactttcataagcagaaaaaatttctattcttgggggaaaagattatgccagatcctctaggattaaatgctgatgcatctgctaaaccttcacatatcagaacatatttactatagaaagaatgaaaatgggacatttgtgtgtcacctatgtgaacattccaaaaatattttacaacaactaagtattttataaattttatgaactgaaatttagttcaagttctaggaaaatacaaaccttgctagatattataaaaatgatacaatatatattcatttcaggctcatcagaatatatctgttatcacttgacaagaatgaaaatgcaccattttgtagtgctttaaaatcaggaagatccagagtactaaaaatgacttcttccttgaagcttactcaccaacttcctcccagttactcactgcttctgccacaagcataaactaggacccagccagaactcccttgaaatatacacttgcaacgattactgcatctatcaaaatggttcagtgcctggctacaggttctgcagatcgactaagaatttgaaaagtcttgtttatttcaaaggaagcccatgtgaattctgcccagagttcatcccagatatgcagtctaagaatacagacagatcagcagagatgtattctaaaacaggaattctggcaatataacaaattgatttccaatcaaaacagatttacataccatacttatgtcaagaagttgttttgttttattgcatcctagattttatttttttgatttatggtttactttaagcataaaaaatttgtcaatacaactcttcccaaaaggcataaacaaaaattcataaaacttgcatcacttgagatacttcaggtatgaattcacaactttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtcgtcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcatttaaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactcctgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagctatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaagcttaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaaggtacctgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgagagctctggaggggcctagaagtggtaaatgaatgaaaacgtggttactctccagatctgcctttcccaaatatggccattcttggctgaatcagaaatcaaaggacaggttattaattactagctctaagttacttaccatttgctgagacagttcagaaatctgactgcatctcctcagagatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctataatcgattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttaaaaaggaaaaaaaaaaaaaagacatgttggccagatgcggtggctcacacctgtaatcccagcactttgggaggctgaggtgggcggatcacccctggcctgcccaacatggtgaaaccccgtctctactaaaaacacaaaaattagctgggcgtggtggcgggcgcctgtaattccagctactcgggaggctgaggcaggagaattgctagaacccaggaggcagagattgcagtgagctgagattgcaccattgcactacagtctgggcaacaaaagtgaaactccatcttaaaaaaaaaaagacaatgttcgtgggtccaaacaagacttaatggaagtgagtctaaaaatgagctatgtgggccaggcgtagtggctcccacctgtaatcccagcactttgggaggccgaagcaggcagatcatgaggtcaggagatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagatttgcatcactgcactcctgcctggtgacagagcaagactccatctcaaaaaaaacaaacaaaaataaaagataaaaatgagctatgtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatgatccgccatctccttggataatttgtgttaactcaggataacagaaaaccaaaattatgagttactgatgcaatattcctaaatgtaaaaataattaaagctaatagtagattcatcttccaatttcatatcagtcttacaaataaactacatatataacttgcttgccttcccttctgagggataaagctgttagaagaattaaaatcagcattcttgactattcaaccaagggagggataaattattactcattctagggacatgggctcataactactacatgtgtaaggacatgaatttacccaatattacaatttttccttttattagtgtgtacagtggaagaatagacatgttcactctggacaaaaaaaaaattatacttatcagttatcagaagcacaatgctgaagacagtagttccataacaatttgaagtatgtgatcgaactagtagattatcttagtagtagtgaattattgtaaatgttagtaatttggcagccactgggcagaaaaataagaattgaggctcaatattgatattaatggtggtgattgacacataaattttatcaagtctacacaatataaaattacagaaaggtagaagagtataccagtacaacttcaacatatcttcactacaagggagtaaaatgacatggcctagttactatctaatgaactgcagaaaactaaaagaaaactccaaggcaactcttctctgctgatctggttggtccttttcctaccttttgcaatacccagatacaaacaatggatagaaaacaaagtagacttgtagtatgcaggtcacagtgctaaattcacagaaagaaacccctgaactgaactgctctatttcctggtggtcacaaagagtaattctggtttacacctacagattgatgtcaatctacaccctgttgataacagtgtggccaaggacaaaaaaaaggtgctccgttttaccaattctgtaaaaaattattggcagggtaagctcggctagggcaggattacatttctaggactaccatccccgaaatttagaagatattatatccacataaagcatatctttcacattaatttgcaaaaatctaaaagctttttcttagctcaagtgtgtccaagtttaccctggcagtttaaaacgatagttacaagcagcatgggttgtatcagacacatttgagggccaatttcatgtaagtgatattgggcaagttacttcaactatctgtgcctccaaggtcatactagtgtttatttacctaaagggtacctgttatgtaactttagggtgtttacattagataatgcctgcaaaatatttacttcaacgcctaaaacatagttaagtattcaataaatacctactattgtcactactaacttaaaagtttagagattaagagcagaatctggggtgagacaaacttaggttcaaatcctagtattgttgggtaatcttgggcaagttacttaacctctctgatttgtgtaatttaaaaaattagttaatatacataacagggcttagaagagtatctagcacatagcaccatttaagcatttgttattgctaacatgcaaacaatttaagggaaagaaattttttaaaaaggaagagggatttgcaaactaaaaacaatgagtatcttatgttcaaagaaaactaacaaacagccagctctagcaataattaaattcactatatactggggcaggcatcacaccccaaagctaaaagcgtctacctaggccaggcacggtggctcatgcctgtaatcccagcactttgggaagcagaggcgggcagatcgcttgagctcaggagttcaagaccagcctggacaacatggcaaaacaccatctctacaaaaaatacaaatattaggccgggcgcagtggctcacgcctgtaatcccagcactttgggaggccaaggcgggtggatcacctgagatcaggagttcgagagtagcctggccaacatggtgaaacctcgtctctattaaaaatacaaaaaattagccaggcatggtggcaggcgcctgtaatcccagctactcagggggatgaggtaggagaatcgcttgaacccgggaggcagaggttgcactgagccgagatcatgccactgtactccagcccgggcaacaagagcgaaactccatctcaaaaaataaataaataaataaataaaataaagtacaaatattagccagggatggtggtgcgcacctgtagtcccagctacttgggaggctgaagtgggagaatcccctgagcctggggagaatcacccgagcccgggaagtcgaggctgcagtgagcagtgattgtgccactgcactccatcctaggtgacagagtgagaccctgtctcaaaaaaaagaaattggcagaattaagtaagttgatgtttagagatgaaaaatcaacattttttcctcagcaactgaataaaaacaacagccactaccatttttttgagtacctatttgtagcctattttttaactggtattactcgagagagagagagctaggttcgagacagagctccttctcttaataactgtatgacctagggtatgtctgttagcctctctgaggcttcaaaggttcctcatctgtaaaatggtaataatcataccattgctacagggctgttttgaagactaattaggactatgtaagtaaacatgatgatggctattattactgttccccgccaggggccatgcaagggttgctgattcacatagactgtcttataatcctctcaataactccaagaggtagccagcacctcagatatacataaaatgacttaagcccagagaggtgaagtaagttgcccacagccacacaactagtaaatagcccaaacaagctggattcccagttagactccgttaatagcactgctctttaccttaagtcattacaatgcctaatatgaaatagaatcgcttctttcttagggttcaagtggttaattatttaatgtattcattcaacaaaccatcatcgaggacctcttacaagccaagtactgtgctaagtgctagagttacggcggtgattcctgcccttaaaaagttttagtgggagaaacaacaggtaaccaggtcattgccaaaacaacaaaaataatcataataaagcaggctaaagcatatttaactggccggggttttgactattttagcaagcatgatcagaacggttgaggagggaggccagcagcttggccggttcaacaaacaagaaaaaaccagtgagggtggagctaagataccagaggctgattacggttaagaatgttcttgaaggtaaggaccagattctcattttctatatcctggggcatcggtcagcatggaatctggattctagcacatgtgaatttcggcttgaaatgacctaatgccttttccctagttccttcgtgtgtcaaatacgcatggttaccgctaccagagctgtagtggggcttcaatgaggccatgagcatctccataaagatgaactacagtgtgtgcaaaactaaaggcaaaacctggtccccacacgccctcccaggtggtcgctttccgtgccgaggcccctccagaggtgccccgagaacctcaccatcgcaccccaaacttccagggaagggcctctcccgagaaagcccccacgcccccaccccgcgccatcattcccgaatctgccctcggcccctccccgcagcacgctcgcaggcggcacatgtcaaccaaaacgccatttccaccttctcttcccacacgcagtcctcttttcccagggctcccccgaggagggacccaccccaaaccccgccattccgtcctccctgccgccctcgcgtgacgtaaagccgaacccgggaaactggccgcccccgcctgcggggttccctgggcccggccgctctagaactagtggatcccaattgaaggcctggtctaaatgactccaaaatcaccacttaattcaagagactgatttccctgagtcaggccccttaaagcagctatttcaatgggacagggaaacaaccctaggatctggattagaatcacttgggggctgccacacccccagggctctgatcctgcccttctcccacacgcacattcacatactgctgcagtgaccttccatttctaatgggttcctgggccatctgtcaggtatagggaatggaaaaggggttggggaggctctgcttcagaaagtttgtgtcaggggctcccagagcctccacagatagatagcaggggtccccaccctaccatggcagctataaatgtgatcaacatttattggcctaggatacagcagttagcaaaatgcctgatgtagttcccactccgtggaggttgcaggctagccaagaagtcatgagttcagcaacccttacgcaccagtgggatgagattggaccaggccgagggtagtcttgggaacactcagcatttgtctgagggccagaagaggctgcttgccctcagacaggaggtcagcatctttattgtagcccatgacacctctacaccattgctcttctggtcttatggaagacatctttgggcctgataacagcggagtctgtgtcccacttgtccaggctggagtgccacatcaggcacactccagttgcagggacagcacagacaagtttcaggaaggctggtggcctccaggaggttaaccttataaggccagattgtaacctagttgaaaaacatacacatgccatgataataaaagaacctaggcaccattacaagagaaaaaatcatttttgtagatacgagcatggattcttgggtgggtcagacacactgggcttgtgctctgactgcactgtctcccctacctgaccttgggtaaaccataagactgctgcatgactcagtgtccaccccaaaaaagtaccggtagatattggccacagtagatatcagctagagtggactctcatgacaatgaggggagatgtattccccatcttaggcacctgggactctaccttccatcttctgctccgtgtctctccatccccaggctcttcagaactcagggagtccagaatgtcagctcccagatttcagccttcagaaaggaaacccattaccgttcagttgaacaaatgttgtctgagccccagatctgggctcagaggccatctaggctatgagacaagaggggaacaaagcaccgtctgcactcactcaccacactcacttgctgtcccaggtcacatccatcgggtagagaatctaagaggctgagctagctcccgccaccagcccagcccaccccacctggccccttccttccttctacaaaatatgcaccacctgtcaaagggtgggcagtgccaggcctgcatacagagcactgagtgtaaaagcagacatggaccctgacctccaggagcttccaattttcttgaagagacaaatcagctggcatttcagtccagtgtgatctgctcttggtgagcacagacctagggagttggggcagcttcccagaagaactgcagtccaggctgagggcagagaaatgaggggaatggcgaggaattggggagcaggggggagctcagtagagagccaagggcgggaggtgagaagtccgtgttgggccaggagctaccctccggtggccacagccgaagtcgaggatgcctttggaactcatccccacttctctctttctgtatgtagccgtccaagaacaagtcacctccaagtgtagccggatcaaggcaagccccccatctagcaagcacttgatgccacccagaactgggcttcttcagaacaatctgagtccaggaatgatcccactcaccaggcaccagagctgcgagggcatgggagtgatctcaccaactctggggaagcggcaaggaattttcacctccagcccccagtgtcccatcctctcacactcaggccagactcccctgggcagacttgactctgtctgccagcatatgcagagccccaaggccaccccaccagaagtgcccctgcctgggttctgtcccagctccctgggcacccagtccttgagtccccaccagctcagacggcctagtgtgccaagaatgcccactgcgttcaacaatgctgcatgggtcacagcggcagcagctgtgaccacagcagtttcggggaaaacacccctcagccaagtggataatagcgttcagcagcactcaccttctggccaggcctgccttcagaggccatctgattgggaggcacaagtgcccgctgcgatgggaacacaagtgcccctggccaacaaccccagcttcagcctgctgggcagccagagcctcaggcagagcccggtacagggcccggtgcctgtagcaaacaccaccaagttcctccagcagggtatggccagctttagtcccctgagccccatacagggcatcgagccaccaagctatgtggctgctgctgccaccgctgctgctgcttctgccgttgctgccagccagttcccaggtccgttcgacagaacggatattccccctgagctgccacctgccgactttttgcgccagccccaacccccactaaatgatctgatttcgtcacctgactgcaatgaggtagatttcattgaagctctcttgaaaggctcctgtgtgagcccagatgaagactgggtgtgcaacttgaggctgatcgacgacattttggaacagcatgctgctgctcaaaatgccacagcccagaattctgggcaagtcacccaggatgctggggcactttaaatctgagcaggatgcccatagaaacccccatggtgacatcactctaggaagtggtgtcgatccatacccgcagttgtctcccgttacaatttgagtggtgttgtcagcccatgcttatccctctctctacctgtgacaaaatggaaagctggtgatttttcaagctacgtgtacatatttgaaaattttgtaaatggttttcctaaacattaatgacagaagtatttatacttcattttgtgactttgtaaataaagcgacggcttttgtttcagtagagttgtgtttactatgcattgttttgtgtttattatacaatgttacaaatatgcagaccgtgttgtttgctccagtgataccttgttaagctaggtggctgagtcgcttatggttttaatgcaatgagcaatgtggatatgaccaagagttgttgtgcaagttgacaaatgccaaatagaaaaccacttggccatttatttctatgttcactaaaaatcctattgccttgtgtgattcttaatctcttttgcgaacctttcagtctccgctagctctttcctaatgagctttacagcagaagctgttttatcgttaagtgccccacagagacactttaccaggaggctgggagagttctccagatttgggagaggcgcagagacagtgtgtgagccgagccctgtctcagcaatccacctggaggagctagagtatcctcctccctttaccattcagaccgagagaaaaagcccagcttgtgtgcaccctcgtggggttaaggcgagctgttcctggtttaaagcctttcagtatttgttttgatgtaaggctctgtggtttgggggggaacatctgtaaacattattagttgatttggggtttgtctttgatggtttctatctgcaattatcgtcatgtatatttaagtgtctgttatagaaaacccacacccactgtcctgtaaacttttctcagtgtccagactttctgtaatcacattttaattgccacctcgtatttcacctctacatttgaaatctggcgtctgtttcaagccagtgtgttttttcttcgttctgtaataaacagccaggagaaaagtgcctctatgtttttatttttcaagggagtattcagtacctacaaacccaagtcaggaagcctgctagtggctttggttctttcagaggctgctcgatgccttgtgtgtcagaaagaaagattcagcagttttgcatcatggcaaagaagcctgttattttggggctcagcccctcattttatagaggatgaaacagagggggatgggaggtcacaaagacaactgccccgggagcaggtgtgggggagacttgccctgagggtctagacgctctgcaccaccgtcctgtctcccttgctgaagaccacacatgcccttctttgaccagaccctgccacctgataggccaggacctggtaggcgggtacccaggtttcatggatggaaccacatctccccaaaagtggggaggtagctactgggatgcacgcctcccgccatgtgctataggagagcagctgaagcaacagttgggatcagatgtagtcacaattgaatgcatcatcacatttatccctctaagtggctgggagagttgatatcctcatccctaaggtacaaaatgttccaatttgatcagtggctttcaggagctgagaaaggcatgtgctctgaggcagagctgttatgtcccgcagagcctaaaaatgctctaagaacatgctccctgccaaaattctcaatggctgtgacaagggacaacgatcgaccaatgggggtggaagcagacctccgcagtccaggggccagagctaggacagaggggtcggagaaagagtcattttcccaacactccagctcttggccagtcctcacacagtcccctcctgcttcctgctgagagagatatcctcataggtctgggtaaagtccttcagtcagctttcattccctgtcaccaactttgtctctgttctccctgcccgtctcaggcagcactcctcaggaaacctctccaagagccagcctcactgcagcgcccactattgtccctctgcctcaagtgtcccatccatgccaggccccaggcaggctgcagctttccctcagggccacaccaaagcacttgggctcagctgtgctgtccccctccatcactgagctcaggggcagcaggggtggggtgccaggaggcccattcacccttctctggctctgtgttggacccacctgcccagccactgctgcttagaacctacccgctgggaaaatgaagccctcccggaggggccacctcaacctgagagcctcacggatcacagttgtccccactcagctctgccagccctcagagacccatagataaaagctgagcttggctcgcagagctggttccatcttccattcccagagggttcaacttcctaccccaaccacacagggaacctcaaggctgagccagtgtgggctgcagtgcagaccagcttcctggacacgtcctgccacctgaccccaggctggcctcactgcccctggcactcctgaccctatcctcattcctcctggcagtgcgtgttctgccattccgctttcccttagctgtcctctcactgtactgtcagcttctccttttccaggtgccccccaggggctttccacatgaccctgtcaccccacagcccatccagcaccaattccagctctctgccacccttcaaaggagtgacagtgccctgcttcacctcccactcacccctcaacccagagcaatctggctccagtcttgcctccttccccctaagtactctagtcacagttccaaattcctcctggtcataaagccaaatgaagcttcctggtcctcagcggacttgccacttcagcagtactggactctctcctcccagaaacctgtttccccttggctcctggagcccacactctgctggaatccttctgcctctctggcctgtagcctggccctctctcccaacctgaggtccattctctcctgctcctccacaagatgttgctccttccattacttcctccctctcaaccaaagctccttcattagctctttatcttctggtttcttcccctgggcagacgaatggattcaagagcctgtggcccagcagcccagcactccaggatctcagcacttcagcatcccagtaccctagcatctcaataccccagcaccccagcaccatagtattccagcaccccattgtccaagcatctcagcactccagcatcccagcaccccaacactccagcagcccagaatctcagcaccctagcactgcagcatctcaggaccccagcacttcagcatcccagcacactagtactccagcatctcggcaccccagcacctaggcatcccaacacccagcaccccagcacttaagcatcccaccactacagtatctcaacactccagcaccccagcaccatagtgttccagcaccccagcatcccaacaccccagcacttaagcatcccaacacctcggcatcccaacaccccagcactgcagcatctcagcaccttagcatcccagtgccctagcatctcaatgctccagcacaccagtactacagtattccagcaccccagcactccagcatctcagcactgcagcactgcagcactccagcatcccaaaatcccagcatcccaacaccccagcagaccagcagaccagcatctcagcaccgcagcatccaaggactatcccagcatcccagcaacccagcacctcagcatcccaacaccccagcatttcagcatggcaacaccccagtaccccagcacttcagcaccccagtatcccagcatctcagcgacccagtatcacaaaacctcagcatcctagcaccccagcaccccagcaccttagcaccttagcatcccagcatctcagcgcctcagcatcttgatattctggctgaggtcagcgtggtgtatctagtcagggtcctaactttcacttcgcagggaaatgctgctggactgggtctcatgttgggctgaagctctctagaccccttgaagacagcataaaagagcttggagacgctgggtgtcccccatggaagagttcactctcatcctgctttgacaacagccttctctggggtccctcacgggcccctctttcttactgcaagtttgtctctgagaagactgtgatgcagaagtcactcagctgcctgtggctcctgaagagctgaaggtggaggcctgtaggcctccctatgagaggcgcagaaaaaaccatgattgctagtggggaggtgctccctctacaacccactccataatctgcccccgcccagctctgaggccagccccaggggaaaatgccagatccccagggaggtgtgtgagacctcaggggctccctcctcccttacagcaggctcaggcccctgggggcctcagggccaaggtctgtgggtaagctactatctctcacttgtcctctagccacaaaagccagggagatctggcaatggacatgaggttctgaagaagcacatatgactggcttcctaatgcgtggttgttcagtgattcaataaacacgcatgggccaggcatggggaaatagacaaacatgatccccaacctctcccagagtgaactgggagggaggagtgttcatccctcaggattacaccagagaaacaaaccagcaggagatatatatggttttggggggtcaagaaagaggaaaaacctggcaaggcaagtccaaaatcataggacaggctgtcaggaagggcagcctggaacctctcaagcaggagctgatgctgcagtccacaggcagaatttcttcttcctcggggaaatctcagctttgttcttaaggcctttcaactgattggctgaggtctgccccttcccccacattctccaggataatcttccttacttaaagtcaactattaatcacagctacaaaatcccttcacagctacacatagatcagtgtttgattgacgaacagcccctacagcctagccaagttgacacataaaactaaccatcacagggggacaaatgatgtaaacacatcaacaaataaaacagtaacaagttaaggtctatggaaaaaacacagaaggggcagagagaaagaaagcaagaaggagagtcccagtttgctagggcttgtgggaagtggggagcagttctctttagctaggatatttgggaaaggcatatctgaaggagtgatatttgagcttagattaaaagatgggaaggagcaagccatgcaaagagctaggatgttccaagcagagacggaacagcaagtgcaaatgtcaggaggaatagaaggaggctggtgggtggggtccagtgagcaagaggagggcaggcaggagaggggatggggaggtgggcaggcccagaccacccagggccctggagactatcctgatccaacaagggaagccttgagtcacttcagtgtccatgtggagaatggacctcagactgaatgagggaggcagtaaggagggcctctacctccagggcttcgccctgtggactgcgcatagacatctccaactcagaaagtctgaaccaaactttccatagttcccccaagtctgggcatcctcctactcagtgaaaggcagccatcacacctccctgccctgctcccggatgccccaaatcctcttggtctccaagtccagaacctgagacttgtccttgatgtttgtctttccctcaccctttctgtattctgggaagatgggtttttttcccccagatgaatctgtaaaacttctgtgatcacaataaaaattctggcagtattattttctggaacatgacaaagtgattcaaaattatttatctggaagactacaaaacaagaatagccaggaaatttctaaaaagaaagaagaaggaggaggagaaagaaggaggaggaaaaggaggagaagaagaaaagaaaaagaaccaagaaagggttctagctctaccaaatattaaaacatatcatgaagctatttaaaacaatatggttgtggatactgaaaaagatgtgaataaagtggaaggaaaataaatagaaatgcacatggggattgagactgtgaaaaaggcagcatctcacatcagtgagggatgttcaacacctggtgttgggaaaactggctagtcatttaaaccaaacaactgggtcctctacctcactcctgacattaagatacatttagatgattcaaagagtaagacagaaaaaataacacgtgaaaacactatcagaaaacaacgtgggccaggtgtggtgggtcacgcctgtaatcccagcactttgggaggccgaggcagacagatcacctgaggtggggagttcaagaccagcctgaccaacatggtgaaatcctgtctctactaaaaatacaaaattagctgagcgtggtggcgcatgcctgtaatcccagctactcaggaggccgaggcaggagaatcacttgaacctgggaggcagaggttgtggtgagccgagatcacgccattgcactccagcctgggcaacaagagtgaaaatccatctaaaaaaaaaaaaaaaagccaaggtggatatttttatagtatcagggtagatcaagcttctccaatcatgacatgaaacccagaaaccataaaagaaaagaatgataaaattgcccacgtaaagtaaaaagcttgcacacagaaaaacaccatacaggttacaagatgagcagcaaaatcagagaaaaaacattgcaattcaggacacacagaggctattgttcctaatatttaaaaataaaagtagtggattgtctacaaaaagatgaagacaagaatttcagaaaaccaaatactgcatgttttcacttacaagtggaagctaaacactgagtacacgtgtacacaaagaatggaaccataggccaggcaccgtggctcacgcctgtaatcccagtactttgcgaggccgaagcgggcggatcacctgaggtgaggagttcgagaccatcctggccaacatggtgaaacccagtctctactaaaaatacaaaaattagccgggcgtggtggtgggtgcctgtaatcccagctactcgggaggctgcggcagtagaatcgcttgaaccctggaggtggaccttgcagtgagccgagatcgcaccactgcactccagcctgggcaacagagtgagactccatctcaaaaaaaaaaaaaaggaatagaacaatagacactggggcctacttgagggaggagggtgaggatcaaaaacctgcctatcaggtactatgcttattacctgggtggtgaaataatctgtacaccaaaccccagtgacatgcaatttaccgatgtaacaaacctgcccatgtacccgctgaacctaaaataaaagttggaaaaaaatatagaaattttctttgtaatagccaaaaactgcaaacagcccaggtgtctattagtagaatgcataaacaaactcgggcatgttcatacaatgtaaaactactcatcaataaaaagtgatacttctcagcaatgaaaagaaactagctactgataccagctacaacatggatggatttcaagtgctttatgatgagagcaagaagccagacacaaaagtgtctatatatatatacagtatatatacgtatatatacacatatatacagtatatatatacatatacatgtatatatatactgtatatatactgtatatatatacacagtatatatatacatatatacagtgtatatatactgtgtatatatacatgtatatatactgtgtatatatacatgtatatatactgtgtatatatacatgtatatatactgtgtatatatacatgtatatatatgtatactgtatatatactgtatatatatatacacatatatacagtatatatatacagtatatactgtatatatacagtatatacgtgtatatatacatatatacagtatatatgtaaatatacatatatacagtatatatgtaaatatacatatatacatgtatatatatacactatatatatacatatatagtgtatatatacatatatacatgtatatatttactatatgattccatttatataaagtgccaaaacagtcaaaaataatctatgtggaaaaaatcaacaaagggatcccccgggctgcaggaattcgatggcgcgccgacgtcgcatgctcctctagactcgaggaattcggtaccccgggttcgaaatcgataagcttggatcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaggatctgcgatcgctgaattctggggactttccactggggactttccactggggactttccactggggactttccactggggactttccactcctgcagcagtggatattcccagaaaactttttggatgcagttggggatttcctctttactggatgtggacaatatcctcctattattcacaggaagcaatccctcctataaaagggcctcagaggaagtagtgttcagctgttcttggctgacttcacatcaaagctcctatactgacctgagacagagccatgaattccgtttttttttttttttttggaaatatgagggtttttccgcttctgacagtggaacggaatgacagcagcacaggctggtgaatgactactttctttataagcaaccaccttgagcctgaaatggcagtcgctagtctctattgccttgctgtggcctcgggatggaaatctgctggggaccctacagtcacctaatctctctccttctcatccttctgtttcattcagaggcagcctgccgcccttctgggaaaagaccctgcaagatgcaagccttcagaatctgggatactaaccagaagaccttttacctgagaaacaaccagctcattgctgggtacttacaaggaccaaatatcaaactagaagaaaagatagacatggtgcctattgaccttcatagtgtgttcttgggcatccacgggggcaagctgtgcctgtcttgtgccaagtctggagatgatatcaagctccagctggaggaagttaacatcactgatctgagcaagaacaaagaagaagacaagcgctttaccttcatccgctctgagaaaggccccaccaccagctttgagtcagctgcctgtccaggatggttcctctgcacaacactagaggctgaccgtcctgtgagcctcaccaacacaccggaagagccccttatagtcacgaagttctacttccaggaagaccaatagtctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatccatttaaatgttagatccggagagctcccaacgcgttgggtcgacggcgcgcctaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttt

SEQ ID NO: 3 - DNA, 29273 bp, synthetic construct
  misc_feature 1..29273: note = helper-dependent adenoviral vector-equine Il-1Ragene
  source 1..29273: mol_type = other DNA
aaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaactggacatttgttctaatctttgaagagtatgaaaaattcctattttaaaggtaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcatttcaattgtgtgacacaggcagactgtggatcaaaagtggtttctggtgcgacttactctcttgagtatacctgcagtcccctttcttaagtgtgttaaaaaaaaagggggatttcttcaattcgccaatactctagctctccatgtgctttctaggaaacaagtgttaacccaccttatttgtcaaacctagctccaaaggacttttgactccccacaaaccgatgtagctcaagagagggtatctgtcaccagtatgtatagtgaaaaaagtatcccaagtcccaacagcaattcctaaaaggagtttatttaaaaaaccacacacacctgtaaaataagtatatatcctccaaggtgactagttttaaaaaaacagtattggctttgatgtaaagtactagtgaatatgttagaaaaatctcactgtaaccaagtgaaatgaaagcaagtatggtttgcagagattcaaagaaaatataagaaaacctactgttgccactaaaaagaatcatatattaaatatactcacacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtatcacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaattttctggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacacactaatattaaaagtgtccaataacatttaaaactatactcatacgttaaaatataaatgtatatatgtacttttgcatatagtatacatgcataggccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatgaggaataaacaccctttccaaatcctcagcataatgattaggtatgcaaaataaatcaaggtcataacctggttcatcatcactaatctgaaaaagaaatatagctgtttcaatgagagcattacaggatacaaacatttgattggattaagatgttaaaaaataaccttagtctatcagagaaatttaggtgtaagatgatattagtaactgttaactttgtaggtatgataatgaattatgtaagaaaacaacaggccgggcgggttggttcacacgtgtaatcccagcactttgggaggctgaggcaggcagactgcctgagctcaggagttcgagaccagcctgggcaacacggtgaaatcccgtctctactaaaaatacaaaaaaattagccgggtgtggtgacacatgcctgtagtcccagctacttgggaggctgaggcaggagaatcacttgaacctgggaggtgaaggttgcagtgagccaagatggcaccacttcactccagcctgggaaacagagcaagactctgtctctgagctgagatggcaccacttcactccagcctgggaaacagagcaagactctgtctcaaaaaaaacaaaacacacaaacaaaaaaacaggctgggcgcggtggctcacgcctgtaatcccagcactttgggaggccgaggcgggtggatcacctgaggtcaggagttccagaccagccttgtcaacatggtgaaacctccccccgccgtctctactaaaaatacaaaaattagccaggcgtggtggcaggagcctgtaatcccagctacttgggaggctgaggcaggagaatcgcttgtacccagaaggcagaggttgcactgagctgagatggcaccattgcactccagcctgggggacaagagcgagatttcgtctttaaaaaacaaaaacaaaacaaaaaaccatgtaactatatgtcttagtcatcttagtcaagaatgtagaagtaaagtgataagatatggaatttcctttaggtcacaaagagaaaaagaaaaattttaaagagctaagacaaacgcagcaaaatctttatatttaataatattctaaacatgggtgatgaacatacgggtattcattatactattctctccacttttgagtatgtttgaaaatttagtaaaacaagttttaacacactgtagtctaacaagataaaatatcacactgaacaggaaaaactggcatggtgtggtggctcacacttgtaatcccagtgctttgggaggctgagacaggagagttgcttgaggccaggagttcaagaccgacatggggaatgtagcaagaccccgtccctacaaaaaactttgtaaaaatttgccaggtatggtggtgcatacctgtagtcccagctactcgggaggcggaggcagaaggaatcacttgagcccaggagtttgaggctgcagtgagctacgatcataccacagcactccagcgtggacaacagagtaagaccctatctcaaaaacaaaacaaaacaaaacaaacaaaaaaaaccacaagaaaaactgctggctgatgcagcggctcatgcctgtaatcccagtattttgggaggcccaggtgggcgtatcacctgaggtcaggagttagagaccagcctggccaacatggtgaaaccccatctctactaaaaatacaaaattagccaggcatgtggcacgcgcctgtagtcccagttactgggaggctgaagcaggaggatcacctgagcccgggaggtggaggttgcagtgagccgagatcacaccactgcactccagcctgggtgacacagcaataccctacctcaaaataaaaaagaaaaagaaaagaaaagttgctgtccccgctaccccaatcccaaatccaaacagcctctctcatctcacagtaagggggaaaaatcacccaaaaaagctaagtgatcttttgaaaacccaaactcttagaagtctaagattattatagtcaactcatgaagtgtcatcataaaagatactctaatattatttaagtagaaccacatattggttgtcttggtatgtctagcccctggcatacaaaatatttaataacactgatatggtacctgtgatgtgaaaatgtactatgagtacagctttataaatactatatatgtacctatatacagaaaaaaatacaacaaaatcataaaagcacttatctttgaaagaggagttacagcaattttatttagttctttattgctttgctatatattctaaatttttttcaatgaatatatatcacttttaaaaaaattcaatggtctttcttataaattatctttggcagcatgcgtttttatatatacatataaaatgtatgggaaatttttaaaggatacattaaattaaagcaaaatatacaaacaaaaaatcagaatacaaaaagataaaaagattgggaagggagggagggagtaaggaggaagggtgggtgggtatagagaaatataccaaataatggtaagaagtggggtcttgacactttctacactttttttaaataaaaaaaatttttttctctctcttttttttttttagagacgaagtctcgctatgttgcccaggctggtcttgaactcctgggatcaagagatcctcctgcctcagcctcccaaggtgcttggattacaggtgtgagccaccacgcctggtcactttctacactttaatatatatattttttcattttcaatgtcatttttattagttaatttataatacccattcaccattatattcaaagtctatttgaagaaataaaccagaaagaatgaaatactctagctcacatgctattcaatactaaattacctttcaaatcacattcaagaagctgatgatttaagctttggcggtttccaataaatattggtcaaaccataattaaatctcaatatatcagttagtacctattgagcatctccttttacaacctaagcattgtattaggtgcttaaatacaagcagcttgacttttaatacatttaaaaatacatatttaagacttaaaatcttatttatggaattcagttatattttgaggtttccagtgctgagaaatttgaggtttgtgctgtctttcagtccccaaagctcagttctgagttctcagactttggtggaacttcatgtattgtcaggttggcccgtaatacctgtgggacaacttcagcccctgtgcacatggccaggaggctggttgcaaacattttcaggtaggtggaccaggacatgcccctggtcatggccaggtggaggcatagtgctatacagcaggcagaagtcaatattgatttgtttttaaagaaacatgtactactttcataagcagaaaaaatttctattcttgggggaaaagattatgccagatcctctaggattaaatgctgatgcatctgctaaaccttcacatatcagaacatatttactatagaaagaatgaaaatgggacatttgtgtgtcacctatgtgaacattccaaaaatattttacaacaactaagtattttataaattttatgaactgaaatttagttcaagttctaggaaaatacaaaccttgctagatattataaaaatgatacaatatatattcatttcaggctcatcagaatatatctgttatcacttgacaagaatgaaaatgcaccattttgtagtgctttaaaatcaggaagatccagagtactaaaaatgacttcttccttgaagcttactcaccaacttcctcccagttactcactgcttctgccacaagcataaactaggacccagccagaactcccttgaaatatacacttgcaacgattactgcatctatcaaaatggttcagtgcctggctacaggttctgcagatcgactaagaatttgaaaagtcttgtttatttcaaaggaagcccatgtgaattctgcccagagttcatcccagatatgcagtctaagaatacagacagatcagcagagatgtattctaaaacaggaattctggcaatataacaaattgatttccaatcaaaacagatttacataccatacttatgtcaagaagttgttttgttttattgcatcctagattttatttttttgatttatggtttactttaagcataaaaaatttgtcaatacaactcttcccaaaaggcataaacaaaaattcataaaacttgcatcacttgagatacttcaggtatgaattcacaactttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtcgtcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcatttaaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactcctgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagctatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaagcttaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaaggtacctgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgagagctctggaggggcctagaagtggtaaatgaatgaaaacgtggttactctccagatctgcctttcccaaatatggccattcttggctgaatcagaaatcaaaggacaggttattaattactagctctaagttacttaccatttgctgagacagttcagaaatctgactgcatctcctcagagatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctataatcgattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttaaaaaggaaaaaaaaaaaaaagacatgttggccagatgcggtggctcacacctgtaatcccagcactttgggaggctgaggtgggcggatcacccctggcctgcccaacatggtgaaaccccgtctctactaaaaacacaaaaattagctgggcgtggtggcgggcgcctgtaattccagctactcgggaggctgaggcaggagaattgctagaacccaggaggcagagattgcagtgagctgagattgcaccattgcactacagtctgggcaacaaaagtgaaactccatcttaaaaaaaaaaagacaatgttcgtgggtccaaacaagacttaatggaagtgagtctaaaaatgagctatgtgggccaggcgtagtggctcccacctgtaatcccagcactttgggaggccgaagcaggcagatcatgaggtcaggagatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagatttgcatcactgcactcctgcctggtgacagagcaagactccatctcaaaaaaaacaaacaaaaataaaagataaaaatgagctatgtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatgatccgccatctccttggataatttgtgttaactcaggataacagaaaaccaaaattatgagttactgatgcaatattcctaaatgtaaaaataattaaagctaatagtagattcatcttccaatttcatatcagtcttacaaataaactacatatataacttgcttgccttcccttctgagggataaagctgttagaagaattaaaatcagcattcttgactattcaaccaagggagggataaattattactcattctagggacatgggctcataactactacatgtgtaaggacatgaatttacccaatattacaatttttccttttattagtgtgtacagtggaagaatagacatgttcactctggacaaaaaaaaaattatacttatcagttatcagaagcacaatgctgaagacagtagttccataacaatttgaagtatgtgatcgaactagtagattatcttagtagtagtgaattattgtaaatgttagtaatttggcagccactgggcagaaaaataagaattgaggctcaatattgatattaatggtggtgattgacacataaattttatcaagtctacacaatataaaattacagaaaggtagaagagtataccagtacaacttcaacatatcttcactacaagggagtaaaatgacatggcctagttactatctaatgaactgcagaaaactaaaagaaaactccaaggcaactcttctctgctgatctggttggtccttttcctaccttttgcaatacccagatacaaacaatggatagaaaacaaagtagacttgtagtatgcaggtcacagtgctaaattcacagaaagaaacccctgaactgaactgctctatttcctggtggtcacaaagagtaattctggtttacacctacagattgatgtcaatctacaccctgttgataacagtgtggccaaggacaaaaaaaaggtgctccgttttaccaattctgtaaaaaattattggcagggtaagctcggctagggcaggattacatttctaggactaccatccccgaaatttagaagatattatatccacataaagcatatctttcacattaatttgcaaaaatctaaaagctttttcttagctcaagtgtgtccaagtttaccctggcagtttaaaacgatagttacaagcagcatgggttgtatcagacacatttgagggccaatttcatgtaagtgatattgggcaagttacttcaactatctgtgcctccaaggtcatactagtgtttatttacctaaagggtacctgttatgtaactttagggtgtttacattagataatgcctgcaaaatatttacttcaacgcctaaaacatagttaagtattcaataaatacctactattgtcactactaacttaaaagtttagagattaagagcagaatctggggtgagacaaacttaggttcaaatcctagtattgttgggtaatcttgggcaagttacttaacctctctgatttgtgtaatttaaaaaattagttaatatacataacagggcttagaagagtatctagcacatagcaccatttaagcatttgttattgctaacatgcaaacaatttaagggaaagaaattttttaaaaaggaagagggatttgcaaactaaaaacaatgagtatcttatgttcaaagaaaactaacaaacagccagctctagcaataattaaattcactatatactggggcaggcatcacaccccaaagctaaaagcgtctacctaggccaggcacggtggctcatgcctgtaatcccagcactttgggaagcagaggcgggcagatcgcttgagctcaggagttcaagaccagcctggacaacatggcaaaacaccatctctacaaaaaatacaaatattaggccgggcgcagtggctcacgcctgtaatcccagcactttgggaggccaaggcgggtggatcacctgagatcaggagttcgagagtagcctggccaacatggtgaaacctcgtctctattaaaaatacaaaaaattagccaggcatggtggcaggcgcctgtaatcccagctactcagggggatgaggtaggagaatcgcttgaacccgggaggcagaggttgcactgagccgagatcatgccactgtactccagcccgggcaacaagagcgaaactccatctcaaaaaataaataaataaataaataaaataaagtacaaatattagccagggatggtggtgcgcacctgtagtcccagctacttgggaggctgaagtgggagaatcccctgagcctggggagaatcacccgagcccgggaagtcgaggctgcagtgagcagtgattgtgccactgcactccatcctaggtgacagagtgagaccctgtctcaaaaaaaagaaattggcagaattaagtaagttgatgtttagagatgaaaaatcaacattttttcctcagcaactgaataaaaacaacagccactaccatttttttgagtacctatttgtagcctattttttaactggtattactcgagagagagagagctaggttcgagacagagctccttctcttaataactgtatgacctagggtatgtctgttagcctctctgaggcttcaaaggttcctcatctgtaaaatggtaataatcataccattgctacagggctgttttgaagactaattaggactatgtaagtaaacatgatgatggctattattactgttccccgccaggggccatgcaagggttgctgattcacatagactgtcttataatcctctcaataactccaagaggtagccagcacctcagatatacataaaatgacttaagcccagagaggtgaagtaagttgcccacagccacacaactagtaaatagcccaaacaagctggattcccagttagactccgttaatagcactgctctttaccttaagtcattacaatgcctaatatgaaatagaatcgcttctttcttagggttcaagtggttaattatttaatgtattcattcaacaaaccatcatcgaggacctcttacaagccaagtactgtgctaagtgctagagttacggcggtgattcctgcccttaaaaagttttagtgggagaaacaacaggtaaccaggtcattgccaaaacaacaaaaataatcataataaagcaggctaaagcatatttaactggccggggttttgactattttagcaagcatgatcagaacggttgaggagggaggccagcagcttggccggttcaacaaacaagaaaaaaccagtgagggtggagctaagataccagaggctgattacggttaagaatgttcttgaaggtaaggaccagattctcattttctatatcctggggcatcggtcagcatggaatctggattctagcacatgtgaatttcggcttgaaatgacctaatgccttttccctagttccttcgtgtgtcaaatacgcatggttaccgctaccagagctgtagtggggcttcaatgaggccatgagcatctccataaagatgaactacagtgtgtgcaaaactaaaggcaaaacctggtccccacacgccctcccaggtggtcgctttccgtgccgaggcccctccagaggtgccccgagaacctcaccatcgcaccccaaacttccagggaagggcctctcccgagaaagcccccacgcccccaccccgcgccatcattcccgaatctgccctcggcccctccccgcagcacgctcgcaggcggcacatgtcaaccaaaacgccatttccaccttctcttcccacacgcagtcctcttttcccagggctcccccgaggagggacccaccccaaaccccgccattccgtcctccctgccgccctcgcgtgacgtaaagccgaacccgggaaactggccgcccccgcctgcggggttccctgggcccggccgctctagaactagtggatcccaattgaaggcctggtctaaatgactccaaaatcaccacttaattcaagagactgatttccctgagtcaggccccttaaagcagctatttcaatgggacagggaaacaaccctaggatctggattagaatcacttgggggctgccacacccccagggctctgatcctgcccttctcccacacgcacattcacatactgctgcagtgaccttccatttctaatgggttcctgggccatctgtcaggtatagggaatggaaaaggggttggggaggctctgcttcagaaagtttgtgtcaggggctcccagagcctccacagatagatagcaggggtccccaccctaccatggcagctataaatgtgatcaacatttattggcctaggatacagcagttagcaaaatgcctgatgtagttcccactccgtggaggttgcaggctagccaagaagtcatgagttcagcaacccttacgcaccagtgggatgagattggaccaggccgagggtagtcttgggaacactcagcatttgtctgagggccagaagaggctgcttgccctcagacaggaggtcagcatctttattgtagcccatgacacctctacaccattgctcttctggtcttatggaagacatctttgggcctgataacagcggagtctgtgtcccacttgtccaggctggagtgccacatcaggcacactccagttgcagggacagcacagacaagtttcaggaaggctggtggcctccaggaggttaaccttataaggccagattgtaacctagttgaaaaacatacacatgccatgataataaaagaacctaggcaccattacaagagaaaaaatcatttttgtagatacgagcatggattcttgggtgggtcagacacactgggcttgtgctctgactgcactgtctcccctacctgaccttgggtaaaccataagactgctgcatgactcagtgtccaccccaaaaaagtaccggtagatattggccacagtagatatcagctagagtggactctcatgacaatgaggggagatgtattccccatcttaggcacctgggactctaccttccatcttctgctccgtgtctctccatccccaggctcttcagaactcagggagtccagaatgtcagctcccagatttcagccttcagaaaggaaacccattaccgttcagttgaacaaatgttgtctgagccccagatctgggctcagaggccatctaggctatgagacaagaggggaacaaagcaccgtctgcactcactcaccacactcacttgctgtcccaggtcacatccatcgggtagagaatctaagaggctgagctagctcccgccaccagcccagcccaccccacctggccccttccttccttctacaaaatatgcaccacctgtcaaagggtgggcagtgccaggcctgcatacagagcactgagtgtaaaagcagacatggaccctgacctccaggagcttccaattttcttgaagagacaaatcagctggcatttcagtccagtgtgatctgctcttggtgagcacagacctagggagttggggcagcttcccagaagaactgcagtccaggctgagggcagagaaatgaggggaatggcgaggaattggggagcaggggggagctcagtagagagccaagggcgggaggtgagaagtccgtgttgggccaggagctaccctccggtggccacagccgaagtcgaggatgcctttggaactcatccccacttctctctttctgtatgtagccgtccaagaacaagtcacctccaagtgtagccggatcaaggcaagccccccatctagcaagcacttgatgccacccagaactgggcttcttcagaacaatctgagtccaggaatgatcccactcaccaggcaccagagctgcgagggcatgggagtgatctcaccaactctggggaagcggcaaggaattttcacctccagcccccagtgtcccatcctctcacactcaggccagactcccctgggcagacttgactctgtctgccagcatatgcagagccccaaggccaccccaccagaagtgcccctgcctgggttctgtcccagctccctgggcacccagtccttgagtccccaccagctcagacggcctagtgtgccaagaatgcccactgcgttcaacaatgctgcatgggtcacagcggcagcagctgtgaccacagcagtttcggggaaaacacccctcagccaagtggataatagcgttcagcagcactcaccttctggccaggcctgccttcagaggccatctgattgggaggcacaagtgcccgctgcgatgggaacacaagtgcccctggccaacaaccccagcttcagcctgctgggcagccagagcctcaggcagagcccggtacagggcccggtgcctgtagcaaacaccaccaagttcctccagcagggtatggccagctttagtcccctgagccccatacagggcatcgagccaccaagctatgtggctgctgctgccaccgctgctgctgcttctgccgttgctgccagccagttcccaggtccgttcgacagaacggatattccccctgagctgccacctgccgactttttgcgccagccccaacccccactaaatgatctgatttcgtcacctgactgcaatgaggtagatttcattgaagctctcttgaaaggctcctgtgtgagcccagatgaagactgggtgtgcaacttgaggctgatcgacgacattttggaacagcatgctgctgctcaaaatgccacagcccagaattctgggcaagtcacccaggatgctggggcactttaaatctgagcaggatgcccatagaaacccccatggtgacatcactctaggaagtggtgtcgatccatacccgcagttgtctcccgttacaatttgagtggtgttgtcagcccatgcttatccctctctctacctgtgacaaaatggaaagctggtgatttttcaagctacgtgtacatatttgaaaattttgtaaatggttttcctaaacattaatgacagaagtatttatacttcattttgtgactttgtaaataaagcgacggcttttgtttcagtagagttgtgtttactatgcattgttttgtgtttattatacaatgttacaaatatgcagaccgtgttgtttgctccagtgataccttgttaagctaggtggctgagtcgcttatggttttaatgcaatgagcaatgtggatatgaccaagagttgttgtgcaagttgacaaatgccaaatagaaaaccacttggccatttatttctatgttcactaaaaatcctattgccttgtgtgattcttaatctcttttgcgaacctttcagtctccgctagctctttcctaatgagctttacagcagaagctgttttatcgttaagtgccccacagagacactttaccaggaggctgggagagttctccagatttgggagaggcgcagagacagtgtgtgagccgagccctgtctcagcaatccacctggaggagctagagtatcctcctccctttaccattcagaccgagagaaaaagcccagcttgtgtgcaccctcgtggggttaaggcgagctgttcctggtttaaagcctttcagtatttgttttgatgtaaggctctgtggtttgggggggaacatctgtaaacattattagttgatttggggtttgtctttgatggtttctatctgcaattatcgtcatgtatatttaagtgtctgttatagaaaacccacacccactgtcctgtaaacttttctcagtgtccagactttctgtaatcacattttaattgccacctcgtatttcacctctacatttgaaatctggcgtctgtttcaagccagtgtgttttttcttcgttctgtaataaacagccaggagaaaagtgcctctatgtttttatttttcaagggagtattcagtacctacaaacccaagtcaggaagcctgctagtggctttggttctttcagaggctgctcgatgccttgtgtgtcagaaagaaagattcagcagttttgcatcatggcaaagaagcctgttattttggggctcagcccctcattttatagaggatgaaacagagggggatgggaggtcacaaagacaactgccccgggagcaggtgtgggggagacttgccctgagggtctagacgctctgcaccaccgtcctgtctcccttgctgaagaccacacatgcccttctttgaccagaccctgccacctgataggccaggacctggtaggcgggtacccaggtttcatggatggaaccacatctccccaaaagtggggaggtagctactgggatgcacgcctcccgccatgtgctataggagagcagctgaagcaacagttgggatcagatgtagtcacaattgaatgcatcatcacatttatccctctaagtggctgggagagttgatatcctcatccctaaggtacaaaatgttccaatttgatcagtggctttcaggagctgagaaaggcatgtgctctgaggcagagctgttatgtcccgcagagcctaaaaatgctctaagaacatgctccctgccaaaattctcaatggctgtgacaagggacaacgatcgaccaatgggggtggaagcagacctccgcagtccaggggccagagctaggacagaggggtcggagaaagagtcattttcccaacactccagctcttggccagtcctcacacagtcccctcctgcttcctgctgagagagatatcctcataggtctgggtaaagtccttcagtcagctttcattccctgtcaccaactttgtctctgttctccctgcccgtctcaggcagcactcctcaggaaacctctccaagagccagcctcactgcagcgcccactattgtccctctgcctcaagtgtcccatccatgccaggccccaggcaggctgcagctttccctcagggccacaccaaagcacttgggctcagctgtgctgtccccctccatcactgagctcaggggcagcaggggtggggtgccaggaggcccattcacccttctctggctctgtgttggacccacctgcccagccactgctgcttagaacctacccgctgggaaaatgaagccctcccggaggggccacctcaacctgagagcctcacggatcacagttgtccccactcagctctgccagccctcagagacccatagataaaagctgagcttggctcgcagagctggttccatcttccattcccagagggttcaacttcctaccccaaccacacagggaacctcaaggctgagccagtgtgggctgcagtgcagaccagcttcctggacacgtcctgccacctgaccccaggctggcctcactgcccctggcactcctgaccctatcctcattcctcctggcagtgcgtgttctgccattccgctttcccttagctgtcctctcactgtactgtcagcttctccttttccaggtgccccccaggggctttccacatgaccctgtcaccccacagcccatccagcaccaattccagctctctgccacccttcaaaggagtgacagtgccctgcttcacctcccactcacccctcaacccagagcaatctggctccagtcttgcctccttccccctaagtactctagtcacagttccaaattcctcctggtcataaagccaaatgaagcttcctggtcctcagcggacttgccacttcagcagtactggactctctcctcccagaaacctgtttccccttggctcctggagcccacactctgctggaatccttctgcctctctggcctgtagcctggccctctctcccaacctgaggtccattctctcctgctcctccacaagatgttgctccttccattacttcctccctctcaaccaaagctccttcattagctctttatcttctggtttcttcccctgggcagacgaatggattcaagagcctgtggcccagcagcccagcactccaggatctcagcacttcagcatcccagtaccctagcatctcaataccccagcaccccagcaccatagtattccagcaccccattgtccaagcatctcagcactccagcatcccagcaccccaacactccagcagcccagaatctcagcaccctagcactgcagcatctcaggaccccagcacttcagcatcccagcacactagtactccagcatctcggcaccccagcacctaggcatcccaacacccagcaccccagcacttaagcatcccaccactacagtatctcaacactccagcaccccagcaccatagtgttccagcaccccagcatcccaacaccccagcacttaagcatcccaacacctcggcatcccaacaccccagcactgcagcatctcagcaccttagcatcccagtgccctagcatctcaatgctccagcacaccagtactacagtattccagcaccccagcactccagcatctcagcactgcagcactgcagcactccagcatcccaaaatcccagcatcccaacaccccagcagaccagcagaccagcatctcagcaccgcagcatccaaggactatcccagcatcccagcaacccagcacctcagcatcccaacaccccagcatttcagcatggcaacaccccagtaccccagcacttcagcaccccagtatcccagcatctcagcgacccagtatcacaaaacctcagcatcctagcaccccagcaccccagcaccttagcaccttagcatcccagcatctcagcgcctcagcatcttgatattctggctgaggtcagcgtggtgtatctagtcagggtcctaactttcacttcgcagggaaatgctgctggactgggtctcatgttgggctgaagctctctagaccccttgaagacagcataaaagagcttggagacgctgggtgtcccccatggaagagttcactctcatcctgctttgacaacagccttctctggggtccctcacgggcccctctttcttactgcaagtttgtctctgagaagactgtgatgcagaagtcactcagctgcctgtggctcctgaagagctgaaggtggaggcctgtaggcctccctatgagaggcgcagaaaaaaccatgattgctagtggggaggtgctccctctacaacccactccataatctgcccccgcccagctctgaggccagccccaggggaaaatgccagatccccagggaggtgtgtgagacctcaggggctccctcctcccttacagcaggctcaggcccctgggggcctcagggccaaggtctgtgggtaagctactatctctcacttgtcctctagccacaaaagccagggagatctggcaatggacatgaggttctgaagaagcacatatgactggcttcctaatgcgtggttgttcagtgattcaataaacacgcatgggccaggcatggggaaatagacaaacatgatccccaacctctcccagagtgaactgggagggaggagtgttcatccctcaggattacaccagagaaacaaaccagcaggagatatatatggttttggggggtcaagaaagaggaaaaacctggcaaggcaagtccaaaatcataggacaggctgtcaggaagggcagcctggaacctctcaagcaggagctgatgctgcagtccacaggcagaatttcttcttcctcggggaaatctcagctttgttcttaaggcctttcaactgattggctgaggtctgccccttcccccacattctccaggataatcttccttacttaaagtcaactattaatcacagctacaaaatcccttcacagctacacatagatcagtgtttgattgacgaacagcccctacagcctagccaagttgacacataaaactaaccatcacagggggacaaatgatgtaaacacatcaacaaataaaacagtaacaagttaaggtctatggaaaaaacacagaaggggcagagagaaagaaagcaagaaggagagtcccagtttgctagggcttgtgggaagtggggagcagttctctttagctaggatatttgggaaaggcatatctgaaggagtgatatttgagcttagattaaaagatgggaaggagcaagccatgcaaagagctaggatgttccaagcagagacggaacagcaagtgcaaatgtcaggaggaatagaaggaggctggtgggtggggtccagtgagcaagaggagggcaggcaggagaggggatggggaggtgggcaggcccagaccacccagggccctggagactatcctgatccaacaagggaagccttgagtcacttcagtgtccatgtggagaatggacctcagactgaatgagggaggcagtaaggagggcctctacctccagggcttcgccctgtggactgcgcatagacatctccaactcagaaagtctgaaccaaactttccatagttcccccaagtctgggcatcctcctactcagtgaaaggcagccatcacacctccctgccctgctcccggatgccccaaatcctcttggtctccaagtccagaacctgagacttgtccttgatgtttgtctttccctcaccctttctgtattctgggaagatgggtttttttcccccagatgaatctgtaaaacttctgtgatcacaataaaaattctggcagtattattttctggaacatgacaaagtgattcaaaattatttatctggaagactacaaaacaagaatagccaggaaatttctaaaaagaaagaagaaggaggaggagaaagaaggaggaggaaaaggaggagaagaagaaaagaaaaagaaccaagaaagggttctagctctaccaaatattaaaacatatcatgaagctatttaaaacaatatggttgtggatactgaaaaagatgtgaataaagtggaaggaaaataaatagaaatgcacatggggattgagactgtgaaaaaggcagcatctcacatcagtgagggatgttcaacacctggtgttgggaaaactggctagtcatttaaaccaaacaactgggtcctctacctcactcctgacattaagatacatttagatgattcaaagagtaagacagaaaaaataacacgtgaaaacactatcagaaaacaacgtgggccaggtgtggtgggtcacgcctgtaatcccagcactttgggaggccgaggcagacagatcacctgaggtggggagttcaagaccagcctgaccaacatggtgaaatcctgtctctactaaaaatacaaaattagctgagcgtggtggcgcatgcctgtaatcccagctactcaggaggccgaggcaggagaatcacttgaacctgggaggcagaggttgtggtgagccgagatcacgccattgcactccagcctgggcaacaagagtgaaaatccatctaaaaaaaaaaaaaaaagccaaggtggatatttttatagtatcagggtagatcaagcttctccaatcatgacatgaaacccagaaaccataaaagaaaagaatgataaaattgcccacgtaaagtaaaaagcttgcacacagaaaaacaccatacaggttacaagatgagcagcaaaatcagagaaaaaacattgcaattcaggacacacagaggctattgttcctaatatttaaaaataaaagtagtggattgtctacaaaaagatgaagacaagaatttcagaaaaccaaatactgcatgttttcacttacaagtggaagctaaacactgagtacacgtgtacacaaagaatggaaccataggccaggcaccgtggctcacgcctgtaatcccagtactttgcgaggccgaagcgggcggatcacctgaggtgaggagttcgagaccatcctggccaacatggtgaaacccagtctctactaaaaatacaaaaattagccgggcgtggtggtgggtgcctgtaatcccagctactcgggaggctgcggcagtagaatcgcttgaaccctggaggtggaccttgcagtgagccgagatcgcaccactgcactccagcctgggcaacagagtgagactccatctcaaaaaaaaaaaaaaggaatagaacaatagacactggggcctacttgagggaggagggtgaggatcaaaaacctgcctatcaggtactatgcttattacctgggtggtgaaataatctgtacaccaaaccccagtgacatgcaatttaccgatgtaacaaacctgcccatgtacccgctgaacctaaaataaaagttggaaaaaaatatagaaattttctttgtaatagccaaaaactgcaaacagcccaggtgtctattagtagaatgcataaacaaactcgggcatgttcatacaatgtaaaactactcatcaataaaaagtgatacttctcagcaatgaaaagaaactagctactgataccagctacaacatggatggatttcaagtgctttatgatgagagcaagaagccagacacaaaagtgtctatatatatatacagtatatatacgtatatatacacatatatacagtatatatatacatatacatgtatatatatactgtatatatactgtatatatatacacagtatatatatacatatatacagtgtatatatactgtgtatatatacatgtatatatactgtgtatatatacatgtatatatactgtgtatatatacatgtatatatactgtgtatatatacatgtatatatatgtatactgtatatatactgtatatatatatacacatatatacagtatatatatacagtatatactgtatatatacagtatatacgtgtatatatacatatatacagtatatatgtaaatatacatatatacagtatatatgtaaatatacatatatacatgtatatatatacactatatatatacatatatagtgtatatatacatatatacatgtatatatttactatatgattccatttatataaagtgccaaaacagtcaaaaataatctatgtggaaaaaatcaacaaagggatcccccgggctgcaggaattcgatggcgcgccgacgtcgcatgctcctctagactcgaggaattcggtaccccgggttcgaaatcgataagcttggatccggagagctcgataacatttaaatggatctaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcaatagcatcacaaatttcacaaataaagcatttttttcactgcattctagttgtggtttgtccaaactcatcaatgtatcttatcatgtcgagctaggatcccgcggccgcctactggtcctcctggaggtagaacttggtgaccatgaaggactctttgggcttgttggtgaggctgacgggccggtctgcctcctgcgccgtgcagaggaaccagccagggcaggcggcagactcgaagctggtggtggggccactgtttgagcggatgaaggtgaagcgcttgttctcctccttgttcttgctcaggtcagtgatgttaactgcctccaattggaacctaatctcatcaccagacttgacacaggccaggcacagcttcctcccatggagtcccaggaatagagcatcaggctcaatgggcaccacatctatcttctcttgtaatttagtatttgattcttgcaagtatccagcaactagttggttattcctcatgtagaaggtcttctggttaacatcccagattctgaaggcttgcatcttgcagggtctcttccccaaagggtggcaggctgtctctgagtagagcaagaaaaggaggagagagattaggtgtctgacagaacgcctgcggatttccataccggtggatccatggctctgtctcaggtcagtataggagctttgatgtgaagtcagccaagaacagctgaacactacttcctctgaggcccttttataggagggattgcttcctgtgaataataggaggatattgtccacatccagtaaagaggaaatccccaactgcatccaaaaagttttctgggaatatccactgctgcaggagtggaaagtccccagtggaaagtccccagtggaaagtccccagtggaaagtccccagtggaaagtccccagaatttcgacggcgcgcctaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttt

SEQ ID NO: 4 - DNA, 534 bp, synthetic construct
  misc_feature 1..534: note = codon optimized human IL-1Ra
  source 1..534: mol_type = other DNA
atggaaatctgcagaggcctgcggagccacctgattaccctgctgctgttcctgttccacagcgagacaatctgccggcccagcggcagaaagtccagcaagatgcaggccttccggatctgggacgtgaaccagaaaaccttctacctgcggaacaatcagctggtggccggctacctgcagggccccaatgtgaacctggaagagaagatcgacgtggtgcccatcgagccccacgccctgtttctgggaatccacggcggcaagatgtgcctgagctgcgtgaagtccggcgacgagacacggctgcagctggaagccgtgaacatcaccgacctgagcgagaaccggaagcaggacaagagattcgccttcatcagaagcgacagcggccccaccaccagctttgagtctgccgcttgccctggctggttcctgtgcactgccatggaagccgaccagcccgtgtccctgaccaacatgcctgacgagggcgtgatggtcaccaagttctattttcaagaggacgagtga

SEQ ID NO: 5 - DNA, 534 bp, synthetic construct
  misc_feature 1..534: note = original human IL-1Ra
  source 1..534: mol_type = other DNA
atggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtag

SEQ ID NO: 6 - protein, 177 aa, Homo sapiens
  REGION 1..177: note = human IL-1Ra protein
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 7 - DNA, 29260 bp, synthetic construct
  misc_feature 1..29260: note = helper-dependent adenoviral vector-human Il-1Ragene
  source 1..29260: mol_type = other DNA
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaactggacatttgttctaatctttgaagagtatgaaaaattcctattttaaagtaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcatttcaattgtgtgacacaggcagactgtggatcaaaagtggtttctggtgcgacttactctcttgagtatacctgcagtcccctttcttaagtgtgttaaaaaaaaagggggatttcttcaattcgccaatactctagctctccatgtgctttctaggaaacaagtgttaacccaccttatttgtcaaacctagctccaaaggacttttgactccccacaaaccgatgtagctcaagagagggtatctgtcaccagtatgtatagtgaaaaaagtatcccaagtcccaacagcaattcctaaaaggagtttatttaaaaaaccacacacacctgtaaaataagtatatatcctccaaggtgactagttttaaaaaaacagtattggctttgatgtaaagtactagtgaatatgttagaaaaatctcactgtaaccaagtgaaatgaaagcaagtatggtttgcagagattcaaagaaaatataagaaaacctactgttgccactaaaaagaatcatatattaaatatactcacacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtaacacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaatttcttggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacacactaatattaaaagtgtccaataacatttaaaactatactcatacgttaaaatataaatgtatatatgtacttttgcatatagtatacatgcatagccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatgaggaataaacaccctttccaaatcctcagcataatgattaggtatgcaaaataaatcaaggtcataacctggttcatcatcactaatctgaaaaagaaatatagctgtttcaatgagagcattacaggatacaaacatttgattggattaagatgttaaaaaataaccttagtctatcagagaaatttaggtgtaagatgatattagtaactgttaactttgtaggtatgataatgaattatgtaagaaaacaacaggccgggcgggttggttcacacgtgtaatcccagcactttgggaggctgaggcaggcagactgcctgagctcaggagttcgagaccagcctgggcaacacggtgaaatcccgtctctactaaaaatacaaaaaaattagccgggtgtggtgacacatgcctgtagtcccagctacttgggaggctgaggcaggagaatcacttgaacctgggaggtgaaggttgcagtgagccaagaatgcgccacttcactccagcctgggaaacagagcaagactctgtctctgagctgagatsgcrccacttcactccagcctgggaaacagagcaagactctgtctcaaaaaaaacaaaacaaacaaacaaaaaaacaggctgggcgcggtggctcacgcctgtaatcccagcactttgggaggccgaggcgggtggatcacctgaggtcaggagttccagaccagccttgtcaacatggtgaaacctccccccgccgtctctactaaaaatacaaaaattagccaggcgtggtggcaggagcctgtaatcccagctacttgggaggctgaggcaggagaatcgcttgtacccagaaggcagaggttgcactgagctgagatggcaccattgcactccagcctgggggacaagagcgagatttcgtctttaaaaaacaaaaaacaaaacaaaaaaccatgtaactatatgtcttagtcatcttagtcaagaatgtagaagtaaagtgataagatatggaatttcctttaggtcacaaagagaaaaagaaaaattttaaagagctaagacaaacgcagcaaaatctttatatttaataatattctaaacatgggtgatgaacatacgggtattcattatactattctctccacttttgagtatgtttgaaaatttagtaaaacaagttttaacacactgtagtctaacaagataaaatatcacactgaacaggaaaaactggcatggtgtggtggctcacacttgtaatcccagtgctttgggaggctgagacaggagagttgcttgaggccaggagttcaagaccgacatggggaatgtagcaagaccccgtccctacaaaaaactttgtaaaaatttgccaggtatggtggtgcatacctgtagtcccagctactcgggaggcggaggcagaaggaatcacttgagcccaggagtttgaggctgcagtgagctacgatcataccacagcactccagcgtggacaacagagtaagaccctatctcaaaaacaaaacaaaacaaaacaaacaaaaaaaaccacaagaaaaactgctggctgatgcagcggctcatgcctgtaatcccagtattttgggaggcccaggtgggcgtatcacctgaggtcaggagttagagaccagcctggccaacatggtgaaaccccatctctactaaaaatacaaaattagccaggcatgtggcacgcgcctgtagtcccagttactggggaggctgaagcaggaggatcacctgagcccgggaggtggaggttgcagtgagccgagatcacaccactgcactccagcctgggtgacacagcaataccctacctcaaaataaaaaagaaaaagaaaagaaaagttgctgtccccgctaccccaatcccaaatccaaacagcctctctcatctcacagtaagggggaaaaatcacccaaaaaagctaagtgatcttttgaaaacccaaactcttagaagtctaagattattatagtcaactcatgaagtgtcatcataaaagatactctaatattatttaagtagaaccacatattggttgtcttggtatgtctagcccctggcatacaaaatatttaataacactgatatggtacctgtgatgtgaaaatgtactatgagtacagctttataaatactatatatgtacctatatacagaaaaaaatacaacaaaatcataaaagcacttatctttgaaagaggagttacagcaattttatttagttctttattgctttgctatatattctaaatttttttcaatgaatatatatcacttttaaaaaaattcaatggtctttcttataaattatctttggcagcatgcgtttttatatatacatataaaatgtatgggaaatttttaaaggatacattaaattaaagcaaaatatacaaacaaaaaatcagaatacaaaaagataaaaagattgggaagggagggagggagtaaggaggaagggtgggtgggtatagagaaatataccaaataatggtaagaagtggggtcttgacactttctacactttttttaaataaaaaaaatttttttctctctcttttttttttttagagacgaagtctcgctatgttgcccaggctggtcttgaactcctgggatcaagagatcctcctgcctcagcctcccaaggtgcttggattacaggtgtgagccaccacgcctggtcactttctacactttaatatatatattttttcattttcaatgtcatttttattagttaatttataatacccattcaccattatattcaaagtctatttgaagaaataaaccagaaagaatgaaatactctagctcacatgctattcaatactaaattacctttcaaatcacattcaagaagctgatgatttaagctttggcggtttccaataaatattggtcaaaccataattaaatctcaatatatcagttagtacctattgagcatctccttttacaacctaagcattgtattaggtgcttaaatacaagcagcttgacttttaatacatttaaaaatacatatttaagacttaaaatcttatttatggaattcagttatattttgaggtttccagtgctgagaaatttgaggtttgtgctgtctttcagtccccaaagctcagttctgagttctcagactttggtggaacttcatgtattgtcaggttggcccgtaatacctgtgggacaacttcagcccctgtgcacatggccaggaggctggttgcaaacattttcaggtaggtggaccaggacatgcccctggtcatggccaggtggaggcatagtgctatacagcaggcagaagtcaatattgatttgtttttaaagaaacatgtactactttcataagcagaaaaaatttctattcttgggggaaaagattatgccagatcctctaggattaaatgctgatgcatctgctaaaccttcacatatcagaacatatttactatagaaagaatgaaaatgggacatttgtgtgtcacctatgtgaacattccaaaaatattttacaacaactaagtattttataaattttatgaactgaaatttagttcaagttctaggaaaatacaaaccttgctagatattataaaaatgatacaatatatattcatttcaggctcatcagaatatatctgttatcacttgacaagaatgaaaatgcaccattttgtagtgctttaaaatcaggaagatccagagtactaaaaatgacttcttccttgaagcttactcaccaacttcctcccagttactcactgcttctgccacaagcataaactaggacccagccagaactccttgaaatatacacttgcaacgattactgcatctatcaaaatggttcagtgcctggctacaggttctgcagatcgactaagaatttgaaaagtcttgtttatttcaaaggaagcccatgtgaattctgcccagagttcatcccagatatgcagtctaagaatacagacagatcagcagagatgtattctaaaacaggaattctggcaatataacaaattgatttccaatcaaaacagatttacataccatacttatgtcaagaagttgttttgttttattgcatcctagattttatttttttgatttatggtttactttaagcataaaaaatttgtcaatacaactcttcccaaaaggcataaacaaaaattcataaaacttgcatcacttgagatacttcaggtatgaattcacaactttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtsgctcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcatttaaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcytggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactccgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagcttatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaagcttaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaaggtacctgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgagagctctggaggggcctagaagtggtaaatgaatgaaaacgtggttactctccagatctgcctttcccaaatatggccattcttggctgaatcagaaatcaaaggacaggttattaattactagctctaagttacttaccatttgctgagacagttcagaaatctgactgcatctcctcagagatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctataatcgattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttaaaaaggaaaaaaaaaaaaaagacatgttggccagatgcggtggctcacacctgtaatcccagcactttgggaggctgaggtgggcggatcacccctggcctgcccaacatggtgaaaccccgtctctactaaaaacacaaaaattagctgggcgtggtggcgggcgcctgtaattccagctactcgggaggctgaggcaggagaattgctagaacccaggaggcagagattgcagtgagctgagattgcaccattgcactacagtctgggcaacaaaagtgaaactccatcttaaaaaaaaaaagacaatgttcgtgggtccaaacaagacttaatggaagtgagtctaaaaatgagctatgtgggccaggcgtagtggctcccacctgtaatcccagcactttgggaggccgaagcaggcagatcatgaggtcaggagatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagattgcatcactgcactcctgcctggtgacagagcaagactccatctcaaaaaaaacaaacaaaaataaaagataaaaatgagctatgtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatgatccgccatctccttggataatttgtgttaactcaggataacagaaaaccaaaattatgagttactgatgcaatattcctaaatgtaaaaataattaaagctaatagtagattcatcttccaatttcatatcagtcttacaaataaactacatatataacttgcttgccttcccttctgagggataaagctgttagaagaattaaaatcagcattcttgactattcaaccaagggagggataaattattactcattctagggacatgggctcataactactacatgtgtaaggacatgaatttacccaatattacaatttttccttttattagtgtgtacagtggaagaatagacatgttcactctggacaaaaaaaaaattatacttatcagttatcagaagcacaatgctgaagacagtagttccataacaatttgaagtatgtgatcgaactagtagattatcttagtagtagtgaattattgtaaatgttagtaatttggcagccactgggcagaaaaataagaattgaggctcaatattgatattaatggtggtgattgacacataaattttatcaagtctacacaatataaaattacagaaaggtagaagagtataccagtacaacttcaacatatcttcactacaagggagtaaaatgacatggcctagttactatctaatgaactgcagaaaactaaaagaaaactccaaggcaactcttctctgctgatctggttggtccttttcctaccttttgcaatacccagatacaaacaatggatagaaaacaaagtagacttgtagtatgcaggtcacagtgctaaattcacagaaagaaacccctgaactgaactgctctatttcctggtggtcacaaagagtaattctggtttacacctacagattgatgtcaatctacaccctgttgataacagtgtggccaaggacaaaaaaaaggtgctccgttttaccaattctgtaaaaaattattggcagggtaagctcggctagggcaggattacatttctaggactaccatccccgaaatttagaagatattatatccacataaagcatatctttcacattaatttgcaaaaatctaaaagctttttcttagctcaagtgtgtccaagtttaccctggcagtttaaaacgatagttacaagcagcatgggttgtatcagacacatttgagggccaatttcatgtaagtgatattgggcaagttacttcaactatctgtgcctccaaggtcatactagtgtttatttacctaaagggtacctgttatgtaactttagggtgtttacattagataatgcctgcaaaatatttacttcaacgcctaaaacatagttaagtattcaataaatacctactattgtcactactaacttaaaagtttagagattaagagcagaatctggggtgagacaaacttaggttcaaatcctagtattgttgggtaatcttgggcaagttacttaacctctctgatttgtgtaatttaaaaaattagttaatatacataacagggcttagaagagtatctagcacatagcaccatttaagcatttgttattgctaacatgcaaacaatttaagggaaagaaattttttaaaaaggaagagggatttgcaaactaaaaacaatgagtatcttatgttcaaagaaaactaacaaacagccagctctagcaataattaaattcactatatactggggcaggcatcacaccccaaagctaaaagcgtctacctaggccaggcacggtggctcatgcctgtaatcccagcactttgggaagcagaggcgggcagatcgcttgagctcaggagttcaagaccagcctggacaacatggcaaaacaccatctctacaaaaaatacaaatattaggccgggcgcagtggctcacgcctgtaatcccagcactttgggaggccaaggcgggtggatcacctgagatcaggagttcgagagtagcctggccaacatggtgaaacctcgtctctattaaaaatacaaaaaattagccaggcatggtggcaggcgcctgtaatcccagctactcagggggatgaggtaggagaatcgcttgaacccgggaggcagaggttgcactgagccgagatcatgccactgtactccagcccgggcaacaagagcgaaactccatctcaaaaaataaataaataaataaataaaataaagtacaaatattagccagggatggtggtgcgcacctgtagtcccagctacttgggaggctgaagtgggagaatcccctgagcctggggagaatcacccgagcccgggaagtcgaggctgcagtgagcagtgattgtgccactgcactccatcctaggtgacagagtgagaccctgtctcaaaaaaaagaaattggcagaattaagtaagttgatgtttagagatgaaaaatcaacattttttcctcagcaactgaataaaaacaacagccactaccatttttttgagtacctatttgtagcctattttttaactggtattactcgagagagagagagctaggttcgagacagagctccttctcttaataactgtatgacctagggtatgtctgttagcctctctgaggcttcaaaggttcctcatctgtaaaatggtaataatcataccattgctacagggctgttttgaagactaattaggactatgtaagtaaacatgatgatggctattattactgttccccgccaggggccatgcaagggttgctgattcacatagactgtcttataatcctctcaataactccaagaggtagccagcacctcagatatacataaaatgacttaagcccagagaggtgaagtaagttgcccacagccacacaactagtaaatagcccaaacaagctggattcccagttagactccgttaatagcactgctctttaccttaagtcattacaatgcctaatatgaaatagaatcgcttctttcttagggttcaagtggttaattatttaatgtattcattcaacaaaccatcatcgaggacctcttacaagccaagtactgtgctaagtgctagagttacggcggtgattcctgcccttaaaaagttttagtgggagaaacaacaggtaaccaggtcattgccaaaacaacaaaaataatcataataaagcaggctaaagcatatttaactggccggggttttgactattttagcaagcatgatcagaacggttgaggagggaggccagcagcttggccggttcaacaaacaagaaaaaaccagtgagggtggagctaagataccagaggctgattacggttaagaatgttcttgaaggtaaggaccagattctcattttctatatcctggggcatcggtcagcatggaatctggattctagcacatgtgaatttcggcttgaaatgacctaatgccttttccctagttccttcgtgtgtcaaatacgcatggttaccgctaccagagctgtagtggggcttcaatgaggccatgagcatctccataaagatgaactacagtgtgtgcaaaactaaaggcaaaacctggtccccacacgccctcccaggtggtcgctttccgtgccgaggcccctccagaggtgccccgagaacctcaccatcgcaccccaaacttccagggaagggcctctcccgagaaagcccccacgcccccaccccgcgccatcattcccgaatctgccctcggcccctccccgcagcacgctcgcaggcggcacatgtcaaccaaaacgccatttccaccttctcttcccacacgcagtcctcttttcccagggctcccccgaggagggacccaccccaaaccccgccattccgtcctccctgccgccctcgcgtgacgtaaagccgaacccgggaaactggccgcccccgcctgcggggttccctgggcccggccgctctagaactagtggatcccaattgaaggcctggtctaaatgactccaaaatcaccacttaattcaagagactgatttccctgagtcaggccccttaaagcagctatttcaatgggacagggaaacaaccctaggatctggattagaatcacttgggggctgccacacccccagggctctgatcctgcccttctcccacacgcacattcacatactgctgcagtgaccttccatttctaatgggttcctgggccatctgtcaggtatagggaatggaaaaggggttggggaggctctgcttcagaaagtttgtgtcaggggctcccagagcctccacagatagatagcaggggtccccaccctaccatggcagctataaatgtgatcaacatttattggcctaggatacagcagttagcaaaatgcctgatgtagttcccactccgtggaggttgcaggctagccaagaagtcatgagttcagcaacccttacgcaccagtgggatgagattggaccaggccgagggtagtcttgggaacactcagcatttgtctgagggccagaagaggctgcttgccctcagacaggaggtcagcatctttattgtagcccatgacacctctacaccattgctcttctggtcttatggaagacatctttgggcctgataacagcggagtctgtgtcccacttgtccaggctggagtgccacatcaggcacactccagttgcagggacagcacagacaagtttcaggaaggctggtggcctccaggaggttaaccttataaggccagattgtaacctagttgaaaaacatacacatgccatgataataaaagaacctaggcaccattacaagagaaaaaatcatttttgtagatacgagcatggattcttgggtgggtcagacacactgggcttgtgctctgactgcactgtctcccctacctgaccttgggtaaaccataagactgctgcatgactcagtgtccaccccaaaaaagtaccggtagatattggccacagtagatatcagctagagtggactctcatgacaatgaggggagatgtattccccatcttaggcacctgggactctaccttccatcttctgctccgtgtctctccatccccaggctcttcagaactcagggagtccagaatgtcagctcccagatttcagccttcagaaaggaaacccattaccgttcagttgaacaaatgttgtctgagccccagatctgggctcagaggccatctaggctatgagacaagaggggaacaaagcaccgtctgcactcactcaccacactcacttgctgtcccaggtcacatccatcgggtagagaatctaagaggctgagctagctcccgccaccagcccagcccaccccacctggccccttccttccttctacaaaatatgcaccacctgtcaaagggtgggcagtgccaggcctgcatacagagcactgagtgtaaaagcagacatggaccctgacctccaggagcttccaattttcttgaagagacaaatcagctggcatttcagtccagtgtgatctgctcttggtgagcacagacctagggagttggggcagcttcccagaagaactgcagtccaggctgagggcagagaaatgaggggaatggcgaggaattggggagcaggggggagctcagtagagagccaagggcgggaggtgagaagtccgtgttgggccaggagctaccctccggtggccacagccgaagtcgaggatgcctttggaactcatccccacttctctctttctgtatgtagccgtccaagaacaagtcacctccaagtgtagccggatcaaggcaagccccccatctagcaagcacttgatgccacccagaactgggcttcttcagaacaatctgagtccaggaatgatcccactcaccaggcaccagagctgcgagggcatgggagtgatctcaccaactctggggaagcggcaaggaattttcacctccagcccccagtgtcccatcctctcacactcaggccagactcccctgggcagacttgactctgtctgccagcatatgcagagccccaaggccaccccaccagaagtgcccctgcctgggttctgtcccagctccctgggcacccagtccttgagtccccaccagctcagacggcctagtgtgccaagaatgcccactgcgttcaacaatgctgcatgggtcacagcggcagcagctgtgaccacagcagtttcggggaaaacacccctcagccaagtggataatagcgttcagcagcactcaccttctggccaggcctgccttcagaggccatctgattgggaggcacaagtgcccgctgcgatgggaacacaagtgcccctggccaacaaccccagcttcagcctgctgggcagccagagcctcaggcagagcccggtacagggcccggtgcctgtagcaaacaccaccaagttcctccagcagggtatggccagctttagtcccctgagccccatacagggcatcgagccaccaagctatgtggctgctgctgccaccgctgctgctgcttctgccgttgctgccagccagttcccaggtccgttcgacagaacggatattccccctgagctgccacctgccgactttttgcgccagccccaacccccactaaatgatctgatttcgtcacctgactgcaatgaggtagatttcattgaagctctcttgaaaggctcctgtgtgagcccagatgaagactgggtgtgcaacttgaggctgatcgacgacattttggaacagcatgctgctgctcaaaatgccacagcccagaattctgggcaagtcacccaggatgctggggcactttaaatctgagcaggatgcccatagaaacccccatggtgacatcactctaggaagtggtgtcgatccatacccgcagttgtctcccgttacaatttgagtggtgttgtcagcccatgcttatccctctctctacctgtgacaaaatggaaagctggtgatttttcaagctacgtgtacatatttgaaaattttgtaaatggttttcctaaacattaatgacagaagtatttatacttcattttgtgactttgtaaataaagcgacggcttttgtttcagtagagttgtgtttactatgcattgttttgtgtttattatacaatgttacaaatatgcagaccgtgttgtttgctccagtgataccttgttaagctaggtggctgagtcgcttatggttttaatgcaatgagcaatgtggatatgaccaagagttgttgtgcaagttgacaaatgccaaatagaaaaccacttggccatttatttctatgttcactaaaaatcctattgccttgtgtgattcttaatctcttttgcgaacctttcagtctccgctagctctttcctaatgagctttacagcagaagctgttttatcgttaagtgccccacagagacactttaccaggaggctgggagagttctccagatttgggagaggcgcagagacagtgtgtgagccgagccctgtctcagcaatccacctggaggagctagagtatcctcctccctttaccattcagaccgagagaaaaagcccagcttgtgtgcaccctcgtggggttaaggcgagctgttcctggtttaaagcctttcagtatttgttttgatgtaaggctctgtggtttgggggggaacatctgtaaacattattagttgatttggggtttgtctttgatggtttctatctgcaattatcgtcatgtatatttaagtgtctgttatagaaaacccacacccactgtcctgtaaacttttctcagtgtccagactttctgtaatcacattttaattgccacctcgtatttcacctctacatttgaaatctggcgtctgtttcaagccagtgtgttttttcttcgttctgtaataaacagccaggagaaaagtgcctctatgtttttatttttcaagggagtattcagtacctacaaacccaagtcaggaagcctgctagtggctttggttctttcagaggctgctcgatgccttgtgtgtcagaaagaaagattcagcagttttgcatcatggcaaagaagcctgttattttggggctcagcccctcattttatagaggatgaaacagagggggatgggaggtcacaaagacaactgccccgggagcaggtgtgggggagacttgccctgagggtctagacgctctgcaccaccgtcctgtctcccttgctgaagaccacacatgcccttctttgaccagaccctgccacctgataggccaggacctggtaggcgggtacccaggtttcatggatggaaccacatctccccaaaagtggggaggtagctactgggatgcacgcctcccgccatgtgctataggagagcagctgaagcaacagttgggatcagatgtagtcacaattgaatgcatcatcacatttatccctctaagtggctgggagagttgatatcctcatccctaaggtacaaaatgttccaatttgatcagtggctttcaggagctgagaaaggcatgtgctctgaggcagagctgttatgtcccgcagagcctaaaaatgctctaagaacatgctccctgccaaaattctcaatggctgtgacaagggacaacgatcgaccaatgggggtggaagcagacctccgcagtccaggggccagagctaggacagaggggtcggagaaagagtcattttcccaacactccagctcttggccagtcctcacacagtcccctcctgcttcctgctgagagagatatcctcataggtctgggtaaagtccttcagtcagctttcattccctgtcaccaactttgtctctgttctccctgcccgtctcaggcagcactcctcaggaaacctctccaagagccagcctcactgcagcgcccactattgtccctctgcctcaagtgtcccatccatgccaggccccaggcaggctgcagctttccctcagggccacaccaaagcacttgggctcagctgtgctgtccccctccatcactgagctcaggggcagcaggggtggggtgccaggaggcccattcacccttctctggctctgtgttggacccacctgcccagccactgctgcttagaacctacccgctgggaaaatgaagccctcccggaggggccacctcaacctgagagcctcacggatcacagttgtccccactcagctctgccagccctcagagacccatagataaaagctgagcttggctcgcagagctggttccatcttccattcccagagggttcaacttcctaccccaaccacacagggaacctcaaggctgagccagtgtgggctgcagtgcagaccagcttcctggacacgtcctgccacctgaccccaggctggcctcactgcccctggcactcctgaccctatcctcattcctcctggcagtgcgtgttctgccattccgctttcccttagctgtcctctcactgtactgtcagcttctccttttccaggtgccccccaggggctttccacatgaccctgtcaccccacagcccatccagcaccaattccagctctctgccacccttcaaaggagtgacagtgccctgcttcacctcccactcacccctcaacccagagcaatctggctccagtcttgcctccttccccctaagtactctagtcacagttccaaattcctcctggtcataaagccaaatgaagcttcctggtcctcagcggacttgccacttcagcagtactggactctctcctcccagaaacctgtttccccttggctcctggagcccacactctgctggaatccttctgcctctctggcctgtagcctggccctctctcccaacctgaggtccattctctcctgctcctccacaagatgttgctccttccattacttcctccctctcaaccaaagctccttcattagctctttatcttctggtttcttcccctgggcagacgaatggattcaagagcctgtggcccagcagcccagcactccaggatctcagcacttcagcatcccagtaccctagcatctcaataccccagcaccccagcaccatagtattccagcaccccattgtccaagcatctcagcactccagcatcccagcaccccaacactccagcagcccagaatctcagcaccctagcactgcagcatctcaggaccccagcacttcagcatcccagcacactagtactccagcatctcggcaccccagcacctaggcatcccaacacccagcaccccagcacttaagcatcccaccactacagtatctcaacactccagcaccccagcaccatagtgttccagcaccccagcatcccaacaccccagcacttaagcatcccaacacctcggcatcccaacaccccagcactgcagcatctcagcaccttagcatcccagtgccctagcatctcaatgctccagcacaccagtactacagtattccagcaccccagcactccagcatctcagcactgcagcactgcagcactccagcatcccaaaatcccagcatcccaacaccccagcagaccagcagaccagcatctcagcaccgcagcatccaaggactatcccagcatcccagcaacccagcacctcagcatcccaacaccccagcatttcagcatggcaacaccccagtaccccagcacttcagcaccccagtatcccagcatctcagcgacccagtatcacaaaacctcagcatcctagcaccccagcaccccagcaccttagcaccttagcatcccagcatctcagcgcctcagcatcttgatattctggctgaggtcagcgtggtgtatctagtcagggtcctaactttcacttcgcagggaaatgctgctggactgggtctcatgttgggctgaagctctctagaccccttgaagacagcataaaagagcttggagacgctgggtgtcccccatggaagagttcactctcatcctgctttgacaacagccttctctggggtccctcacgggcccctctttcttactgcaagtttgtctctgagaagactgtgatgcagaagtcactcagctgcctgtggctcctgaagagctgaaggtggaggcctgtaggcctccctatgagaggcgcagaaaaaaccatgattgctagtggggaggtgctccctctacaacccactccataatctgcccccgcccagctctgaggccagccccaggggaaaatgccagatccccagggaggtgtgtgagacctcaggggctccctcctcccttacagcaggctcaggcccctgggggcctcagggccaaggtctgtgggtaagctactatctctcacttgtcctctagccacaaaagccagggagatctggcaatggacatgaggttctgaagaagcacatatgactggcttcctaatgcgtggttgttcagtgattcaataaacacgcatgggccaggcatggggaaatagacaaacatgatccccaacctctcccagagtgaactgggagggaggagtgttcatccctcaggattacaccagagaaacaaaccagcaggagatatatatggttttggggggtcaagaaagaggaaaaacctggcaaggcaagtccaaaatcataggacaggctgtcaggaagggcagcctggaacctctcaagcaggagctgatgctgcagtccacaggcagaatttcttcttcctcggggaaatctcagctttgttcttaaggcctttcaactgattggctgaggtctgccccttcccccacattctccaggataatcttccttacttaaagtcaactattaatcacagctacaaaatcccttcacagctacacatagatcagtgtttgattgacgaacagcccctacagcctagccaagttgacacataaaactaaccatcacagggggacaaatgatgtaaacacatcaacaaataaaacagtaacaagttaaggtctatggaaaaaacacagaaggggcagagagaaagaaagcaagaaggagagtcccagtttgctagggcttgtgggaagtggggagcagttctctttagctaggatatttgggaaaggcatatctgaaggagtgatatttgagcttagattaaaagatgggaaggagcaagccatgcaaagagctaggatgttccaagcagagacggaacagcaagtgcaaatgtcaggaggaatagaaggaggctggtgggtggggtccagtgagcaagaggagggcaggcaggagaggggatggggaggtgggcaggcccagaccacccagggccctggagactatcctgatccaacaagggaagccttgagtcacttcagtgtccatgtggagaatggacctcagactgaatgagggaggcagtaaggagggcctctacctccagggcttcgccctgtggactgcgcatagacatctccaactcagaaagtctgaaccaaactttccatagttcccccaagtctgggcatcctcctactcagtgaaaggcagccatcacacctccctgccctgctcccggatgccccaaatcctcttggtctccaagtccagaacctgagacttgtccttgatgtttgtctttccctcaccctttctgtattctgggaagatgggtttttttcccccagatgaatctgtaaaacttctgtgatcacaataaaaattctggcagtattattttctggaacatgacaaagtgattcaaaattatttatctggaagactacaaaacaagaatagccaggaaatttctaaaaagaaagaagaaggaggaggagaaagaaggaggaggaaaaggaggagaagaagaaaagaaaaagaaccaagaaagggttctagctctaccaaatattaaaacatatcatgaagctatttaaaacaatatggttgtggatactgaaaaagatgtgaataaagtggaaggaaaataaatagaaatgcacatggggattgagactgtgaaaaaggcagcatctcacatcagtgagggatgttcaacacctggtgttgggaaaactggctagtcatttaaaccaaacaactgggtcctctacctcactcctgacattaagatacatttagatgattcaaagagtaagacagaaaaaataacacgtgaaaacactatcagaaaacaacgtgggccaggtgtggtgggtcacgcctgtaatcccagcactttgggaggccgaggcagacagatcacctgaggtggggagttcaagaccagcctgaccaacatggtgaaatcctgtctctactaaaaatacaaaattagctgagcgtggtggcgcatgcctgtaatcccagctactcaggaggccgaggcaggagaatcacttgaacctgggaggcagaggttgtggtgagccgagatcacgccattgcactccagcctgggcaacaagagtgaaaatccatctaaaaaaaaaaaaaaaagccaaggtggatatttttatagtatcagggtagatcaagcttctccaatcatgacatgaaacccagaaaccataaaagaaaagaatgataaaattgcccacgtaaagtaaaaagcttgcacacagaaaaacaccatacaggttacaagatgagcagcaaaatcagagaaaaaacattgcaattcaggacacacagaggctattgttcctaatatttaaaaataaaagtagtggattgtctacaaaaagatgaagacaagaatttcagaaaaccaaatactgcatgttttcacttacaagtggaagctaaacactgagtacacgtgtacacaaagaatggaaccataggccaggcaccgtggctcacgcctgtaatcccagtactttgcgaggccgaagcgggcggatcacctgaggtgaggagttcgagaccatcctggccaacatggtgaaacccagtctctactaaaaatacaaaaattagccgggcgtggtggtgggtgcctgtaatcccagctactcgggaggctgcggcagtagaatcgcttgaaccctggaggtggaccttgcagtgagccgagatcgcaccactgcactccagcctgggcaacagagtgagactccatctcaaaaaaaaaaaaaaggaatagaacaatagacactggggcctacttgagggaggagggtgaggatcaaaaacctgcctatcaggtactatgcttattacctgggtggtgaaataatctgtacaccaaaccccagtgacatgcaatttaccgatgtaacaaacctgcccatgtacccgctgaacctaaaataaaagttggaaaaaaatatagaaattttctttgtaatagccaaaaactgcaaacagcccaggtgtctattagtagaatgcataaacaaactcgggcatgttcatacaatgtaaaactactcatcaataaaaagtgatacttctcagcaatgaaaagaaactagctactgataccagctacaacatggatggatttcaagtgctttatgatgagagcaagaagccagacacaaaagtgtctatatatatatacagtatatatacgtatatatacacatatatacagtatatatatacatatacatgtatatatatactgtatatatactgtatatatatacacagtatatatatacatatatacagtgtatatatactgtgtatatatacatgtatatatactgagtatatatacatgtatatatactgtgtatatatacatgtatatatactgagtatatatacatgtatatatatgtatactgtatatatactgtatatatatatacacatatatacagtatatatatacagtatatactgtatatatacagtatatacgtgtatatatacatatatacagtatatatgtaaatatacatatatacagtatatatgtaaatatacatatatacatgtatatatatacactatatatatacatatatagtgtatatatacatatatacatgtatatatttactatatgattccatttatataaagtgccaaaacagtcaaaaataatctatgtggaaaaaatcaacaaagggatcccccgggctgcaggaattcgatggcgcgccgacgcgcatgctcctctagactcgaggaattcggtaccccgggttcgaaatcgataagcttggatccggagagctcgttaacatttaaatggatctaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcaatagcatcacaaatttcacaaataaagcatttttttcactgcattctagttgtggtttgtccaaactcatcaatgtatcttatcatgtcgagatccgcgcggccgctcactcgtcctcttgaaaatagaacttggtgaccatcacgccctcgtcaggcatgttggtcagggacacgggctggtcggcttccatggcagtgcacaggaaccagccagggcaagcggcagactcaaagctggtggtggggccgctgtcgcttctgatgaaggcgaatctcttgtcctgcttccggttctcgctcaggtcggtgatgttcacggcttccagctgcagccgtgtctcgtcgccggacttcacgcagctcaggcacatcttgccgccgtggattcccagaaacagggcgtggggctcgatgggcaccacgtcgatcttctcttccaggttcacattggggccctgcaggtagccggccaccagctgattgttccgcaggtagaaggttttctggttcacgtcccagatccggaaggcctgcatcttgctggactttctgccgctgggccggcagattgtctcgctgtggaacaggaacagcagcagggtaatcaggtggctccgcaggcctctgcagatttccataccggtggatcatggctctgtctcaggtcagtataggagctttgatgtgaagtcagccaagaacagctgaacactacttctgctgaggcccttttataggagggattgcttcctgtgaataataggaggatattgtccacatccagtaaagaggaaatccccaactgcatccaaaaagttttctgggaatatccactgctgcaggagtggaaagtccccagtggaaagtccccagtggaaagtccccagtggaaagtccccagtggaaagtccccagaatttcgacggcgcgcctaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 8 - DNA, 33136 bp, synthetic construct
  misc_feature 1..33136: note = HDV with human PRG4
  source 1..33136: mol_type = other DNA
aaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaactggacatttgttctaatctttgaagagtatgaaaaattcctattttaaaggtaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcatttcaattgtgtgacacaggcagactgtggatcaaaagtggtttctggtgcgacttactctcttgagtatacctgcagtcccctttcttaagtgtgttaaaaaaaaagggggatttcttcaattcgccaatactctagctctccatgtgctttctaggaaacaagtgttaacccaccttatttgtcaaacctagctccaaaggacttttgactccccacaaaccgatgtagctcaagagagggtatctgtcaccagtatgtatagtgaaaaaagtatcccaagtcccaacagcaattcctaaaaggagtttatttaaaaaaccacacacacctgtaaaataagtatatatcctccaaggtgactagttttaaaaaaacagtattggctttgatgtaaagtactagtgaatatgttagaaaaatctcactgtaaccaagtgaaatgaaagcaagtatggtttgcagagattcaaagaaaatataagaaaacctactgttgccactaaaaagaatcatatattaaatatactcacacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtatcacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaattttctggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacacactaatattaaaagtgtccaataacatttaaaactatactcatacgttaaaatataaatgtatatatgtacttttgcatatagtatacatgcataggccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatgaggaataaacaccctttccaaatcctcagcataatgattaggtatgcaaaataaatcaaggtcataacctggttcatcatcactaatctgaaaaagaaatatagctgtttcaatgagagcattacaggatacaaacatttgattggattaagatgttaaaaaataaccttagtctatcagagaaatttaggtgtaagatgatattagtaactgttaactttgtaggtatgataatgaattatgtaagaaaacaacaggccgggcgggttggttcacacgtgtaatcccagcactttgggaggctgaggcaggcagactgcctgagctcaggagttcgagaccagcctgggcaacacggtgaaatcccgtctctactaaaaatacaaaaaaattagccgggtgtggtgacacatgcctgtagtcccagctacttgggaggctgaggcaggagaatcacttgaacctgggaggtgaaggttgcagtgagccaagatggcaccacttcactccagcctgggaaacagagcaagactctgtctctgagctgagatggcaccacttcactccagcctgggaaacagagcaagactctgtctcaaaaaaaacaaaacacacaaacaaaaaaacaggctgggcgcggtggctcacgcctgtaatcccagcactttgggaggccgaggcgggtggatcacctgaggtcaggagttccagaccagccttgtcaacatggtgaaacctccccccgccgtctctactaaaaatacaaaaattagccaggcgtggtggcaggagcctgtaatcccagctacttgggaggctgaggcaggagaatcgcttgtacccagaaggcagaggttgcactgagctgagatggcaccattgcactccagcctgggggacaagagcgagatttcgtctttaaaaaacaaaaacaaaacaaaaaaccatgtaactatatgtcttagtcatcttagtcaagaatgtagaagtaaagtgataagatatggaatttcctttaggtcacaaagagaaaaagaaaaattttaaagagctaagacaaacgcagcaaaatctttatatttaataatattctaaacatgggtgatgaacatacgggtattcattatactattctctccacttttgagtatgtttgaaaatttagtaaaacaagttttaacacactgtagtctaacaagataaaatatcacactgaacaggaaaaactggcatggtgtggtggctcacacttgtaatcccagtgctttgggaggctgagacaggagagttgcttgaggccaggagttcaagaccgacatggggaatgtagcaagaccccgtccctacaaaaaactttgtaaaaatttgccaggtatggtggtgcatacctgtagtcccagctactcgggaggcggaggcagaaggaatcacttgagcccaggagtttgaggctgcagtgagctacgatcataccacagcactccagcgtggacaacagagtaagaccctatctcaaaaacaaaacaaaacaaaacaaacaaaaaaaaccacaagaaaaactgctggctgatgcagcggctcatgcctgtaatcccagtattttgggaggcccaggtgggcgtatcacctgaggtcaggagttagagaccagcctggccaacatggtgaaaccccatctctactaaaaatacaaaattagccaggcatgtggcacgcgcctgtagtcccagttactgggaggctgaagcaggaggatcacctgagcccgggaggtggaggttgcagtgagccgagatcacaccactgcactccagcctgggtgacacagcaataccctacctcaaaataaaaaagaaaaagaaaagaaaagttgctgtccccgctaccccaatcccaaatccaaacagcctctctcatctcacagtaagggggaaaaatcacccaaaaaagctaagtgatcttttgaaaacccaaactcttagaagtctaagattattatagtcaactcatgaagtgtcatcataaaagatactctaatattatttaagtagaaccacatattggttgtcttggtatgtctagcccctggcatacaaaatatttaataacactgatatggtacctgtgatgtgaaaatgtactatgagtacagctttataaatactatatatgtacctatatacagaaaaaaatacaacaaaatcataaaagcacttatctttgaaagaggagttacagcaattttatttagttctttattgctttgctatatattctaaatttttttcaatgaatatatatcacttttaaaaaaattcaatggtctttcttataaattatctttggcagcatgcgtttttatatatacatataaaatgtatgggaaatttttaaaggatacattaaattaaagcaaaatatacaaacaaaaaatcagaatacaaaaagataaaaagattgggaagggagggagggagtaaggaggaagggtgggtgggtatagagaaatataccaaataatggtaagaagtggggtcttgacactttctacactttttttaaataaaaaaaatttttttctctctcttttttttttttagagacgaagtctcgctatgttgcccaggctggtcttgaactcctgggatcaagagatcctcctgcctcagcctcccaaggtgcttggattacaggtgtgagccaccacgcctggtcactttctacactttaatatatatattttttcattttcaatgtcatttttattagttaatttataatacccattcaccattatattcaaagtctatttgaagaaataaaccagaaagaatgaaatactctagctcacatgctattcaatactaaattacctttcaaatcacattcaagaagctgatgatttaagctttggcggtttccaataaatattggtcaaaccataattaaatctcaatatatcagttagtacctattgagcatctccttttacaacctaagcattgtattaggtgcttaaatacaagcagcttgacttttaatacatttaaaaatacatatttaagacttaaaatcttatttatggaattcagttatattttgaggtttccagtgctgagaaatttgaggtttgtgctgtctttcagtccccaaagctcagttctgagttctcagactttggtggaacttcatgtattgtcaggttggcccgtaatacctgtgggacaacttcagcccctgtgcacatggccaggaggctggttgcaaacattttcaggtaggtggaccaggacatgcccctggtcatggccaggtggaggcatagtgctatacagcaggcagaagtcaatattgatttgtttttaaagaaacatgtactactttcataagcagaaaaaatttctattcttgggggaaaagattatgccagatcctctaggattaaatgctgatgcatctgctaaaccttcacatatcagaacatatttactatagaaagaatgaaaatgggacatttgtgtgtcacctatgtgaacattccaaaaatattttacaacaactaagtattttataaattttatgaactgaaatttagttcaagttctaggaaaatacaaaccttgctagatattataaaaatgatacaatatatattcatttcaggctcatcagaatatatctgttatcacttgacaagaatgaaaatgcaccattttgtagtgctttaaaatcaggaagatccagagtactaaaaatgacttcttccttgaagcttactcaccaacttcctcccagttactcactgcttctgccacaagcataaactaggacccagccagaactcccttgaaatatacacttgcaacgattactgcatctatcaaaatggttcagtgcctggctacaggttctgcagatcgactaagaatttgaaaagtcttgtttatttcaaaggaagcccatgtgaattctgcccagagttcatcccagatatgcagtctaagaatacagacagatcagcagagatgtattctaaaacaggaattctggcaatataacaaattgatttccaatcaaaacagatttacataccatacttatgtcaagaagttgttttgttttattgcatcctagattttatttttttgatttatggtttactttaagcataaaaaatttgtcaatacaactcttcccaaaaggcataaacaaaaattcataaaacttgcatcacttgagatacttcaggtatgaattcacaactttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtcgtcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcatttaaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactcctgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagctatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaagcttaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaaggtacctgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgagagctctggaggggcctagaagtggtaaatgaatgaaaacgtggttactctccagatctgcctttcccaaatatggccattcttggctgaatcagaaatcaaaggacaggttattaattactagctctaagttacttaccatttgctgagacagttcagaaatctgactgcatctcctcagagatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctataatcgattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttaaaaaggaaaaaaaaaaaaaagacatgttggccagatgcggtggctcacacctgtaatcccagcactttgggaggctgaggtgggcggatcacccctggcctgcccaacatggtgaaaccccgtctctactaaaaacacaaaaattagctgggcgtggtggcgggcgcctgtaattccagctactcgggaggctgaggcaggagaattgctagaacccaggaggcagagattgcagtgagctgagattgcaccattgcactacagtctgggcaacaaaagtgaaactccatcttaaaaaaaaaaagacaatgttcgtgggtccaaacaagacttaatggaagtgagtctaaaaatgagctatgtgggccaggcgtagtggctcccacctgtaatcccagcactttgggaggccgaagcaggcagatcatgaggtcaggagatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagatttgcatcactgcactcctgcctggtgacagagcaagactccatctcaaaaaaaacaaacaaaaataaaagataaaaatgagctatgtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatgatccgccatctccttggataatttgtgttaactcaggataacagaaaaccaaaattatgagttactgatgcaatattcctaaatgtaaaaataattaaagctaatagtagattcatcttccaatttcatatcagtcttacaaataaactacatatataacttgcttgccttcccttctgagggataaagctgttagaagaattaaaatcagcattcttgactattcaaccaagggagggataaattattactcattctagggacatgggctcataactactacatgtgtaaggacatgaatttacccaatattacaatttttccttttattagtgtgtacagtggaagaatagacatgttcactctggacaaaaaaaaaattatacttatcagttatcagaagcacaatgctgaagacagtagttccataacaatttgaagtatgtgatcgaactagtagattatcttagtagtagtgaattattgtaaatgttagtaatttggcagccactgggcagaaaaataagaattgaggctcaatattgatattaatggtggtgattgacacataaattttatcaagtctacacaatataaaattacagaaaggtagaagagtataccagtacaacttcaacatatcttcactacaagggagtaaaatgacatggcctagttactatctaatgaactgcagaaaactaaaagaaaactccaaggcaactcttctctgctgatctggttggtccttttcctaccttttgcaatacccagatacaaacaatggatagaaaacaaagtagacttgtagtatgcaggtcacagtgctaaattcacagaaagaaacccctgaactgaactgctctatttcctggtggtcacaaagagtaattctggtttacacctacagattgatgtcaatctacaccctgttgataacagtgtggccaaggacaaaaaaaaggtgctccgttttaccaattctgtaaaaaattattggcagggtaagctcggctagggcaggattacatttctaggactaccatccccgaaatttagaagatattatatccacataaagcatatctttcacattaatttgcaaaaatctaaaagctttttcttagctcaagtgtgtccaagtttaccctggcagtttaaaacgatagttacaagcagcatgggttgtatcagacacatttgagggccaatttcatgtaagtgatattgggcaagttacttcaactatctgtgcctccaaggtcatactagtgtttatttacctaaagggtacctgttatgtaactttagggtgtttacattagataatgcctgcaaaatatttacttcaacgcctaaaacatagttaagtattcaataaatacctactattgtcactactaacttaaaagtttagagattaagagcagaatctggggtgagacaaacttaggttcaaatcctagtattgttgggtaatcttgggcaagttacttaacctctctgatttgtgtaatttaaaaaattagttaatatacataacagggcttagaagagtatctagcacatagcaccatttaagcatttgttattgctaacatgcaaacaatttaagggaaagaaattttttaaaaaggaagagggatttgcaaactaaaaacaatgagtatcttatgttcaaagaaaactaacaaacagccagctctagcaataattaaattcactatatactggggcaggcatcacaccccaaagctaaaagcgtctacctaggccaggcacggtggctcatgcctgtaatcccagcactttgggaagcagaggcgggcagatcgcttgagctcaggagttcaagaccagcctggacaacatggcaaaacaccatctctacaaaaaatacaaatattaggccgggcgcagtggctcacgcctgtaatcccagcactttgggaggccaaggcgggtggatcacctgagatcaggagttcgagagtagcctggccaacatggtgaaacctcgtctctattaaaaatacaaaaaattagccaggcatggtggcaggcgcctgtaatcccagctactcagggggatgaggtaggagaatcgcttgaacccgggaggcagaggttgcactgagccgagatcatgccactgtactccagcccgggcaacaagagcgaaactccatctcaaaaaataaataaataaataaataaaataaagtacaaatattagccagggatggtggtgcgcacctgtagtcccagctacttgggaggctgaagtgggagaatcccctgagcctggggagaatcacccgagcccgggaagtcgaggctgcagtgagcagtgattgtgccactgcactccatcctaggtgacagagtgagaccctgtctcaaaaaaaagaaattggcagaattaagtaagttgatgtttagagatgaaaaatcaacattttttcctcagcaactgaataaaaacaacagccactaccatttttttgagtacctatttgtagcctattttttaactggtattactcgagagagagagagctaggttcgagacagagctccttctcttaataactgtatgacctagggtatgtctgttagcctctctgaggcttcaaaggttcctcatctgtaaaatggtaataatcataccattgctacagggctgttttgaagactaattaggactatgtaagtaaacatgatgatggctattattactgttccccgccaggggccatgcaagggttgctgattcacatagactgtcttataatcctctcaataactccaagaggtagccagcacctcagatatacataaaatgacttaagcccagagaggtgaagtaagttgcccacagccacacaactagtaaatagcccaaacaagctggattcccagttagactccgttaatagcactgctctttaccttaagtcattacaatgcctaatatgaaatagaatcgcttctttcttagggttcaagtggttaattatttaatgtattcattcaacaaaccatcatcgaggacctcttacaagccaagtactgtgctaagtgctagagttacggcggtgattcctgcccttaaaaagttttagtgggagaaacaacaggtaaccaggtcattgccaaaacaacaaaaataatcataataaagcaggctaaagcatatttaactggccggggttttgactattttagcaagcatgatcagaacggttgaggagggaggccagcagcttggccggttcaacaaacaagaaaaaaccagtgagggtggagctaagataccagaggctgattacggttaagaatgttcttgaaggtaaggaccagattctcattttctatatcctggggcatcggtcagcatggaatctggattctagcacatgtgaatttcggcttgaaatgacctaatgccttttccctagttccttcgtgtgtcaaatacgcatggttaccgctaccagagctgtagtggggcttcaatgaggccatgagcatctccataaagatgaactacagtgtgtgcaaaactaaaggcaaaacctggtccccacacgccctcccaggtggtcgctttccgtgccgaggcccctccagaggtgccccgagaacctcaccatcgcaccccaaacttccagggaagggcctctcccgagaaagcccccacgcccccaccccgcgccatcattcccgaatctgccctcggcccctccccgcagcacgctcgcaggcggcacatgtcaaccaaaacgccatttccaccttctcttcccacacgcagtcctcttttcccagggctcccccgaggagggacccaccccaaaccccgccattccgtcctccctgccgccctcgcgtgacgtaaagccgaacccgggaaactggccgcccccgcctgcggggttccctgggcccggccgctctagaactagtggatcccaattgaaggcctggtctaaatgactccaaaatcaccacttaattcaagagactgatttccctgagtcaggccccttaaagcagctatttcaatgggacagggaaacaaccctaggatctggattagaatcacttgggggctgccacacccccagggctctgatcctgcccttctcccacacgcacattcacatactgctgcagtgaccttccatttctaatgggttcctgggccatctgtcaggtatagggaatggaaaaggggttggggaggctctgcttcagaaagtttgtgtcaggggctcccagagcctccacagatagatagcaggggtccccaccctaccatggcagctataaatgtgatcaacatttattggcctaggatacagcagttagcaaaatgcctgatgtagttcccactccgtggaggttgcaggctagccaagaagtcatgagttcagcaacccttacgcaccagtgggatgagattggaccaggccgagggtagtcttgggaacactcagcatttgtctgagggccagaagaggctgcttgccctcagacaggaggtcagcatctttattgtagcccatgacacctctacaccattgctcttctggtcttatggaagacatctttgggcctgataacagcggagtctgtgtcccacttgtccaggctggagtgccacatcaggcacactccagttgcagggacagcacagacaagtttcaggaaggctggtggcctccaggaggttaaccttataaggccagattgtaacctagttgaaaaacatacacatgccatgataataaaagaacctaggcaccattacaagagaaaaaatcatttttgtagatacgagcatggattcttgggtgggtcagacacactgggcttgtgctctgactgcactgtctcccctacctgaccttgggtaaaccataagactgctgcatgactcagtgtccaccccaaaaaagtaccggtagatattggccacagtagatatcagctagagtggactctcatgacaatgaggggagatgtattccccatcttaggcacctgggactctaccttccatcttctgctccgtgtctctccatccccaggctcttcagaactcagggagtccagaatgtcagctcccagatttcagccttcagaaaggaaacccattaccgttcagttgaacaaatgttgtctgagccccagatctgggctcagaggccatctaggctatgagacaagaggggaacaaagcaccgtctgcactcactcaccacactcacttgctgtcccaggtcacatccatcgggtagagaatctaagaggctgagctagctcccgccaccagcccagcccaccccacctggccccttccttccttctacaaaatatgcaccacctgtcaaagggtgggcagtgccaggcctgcatacagagcactgagtgtaaaagcagacatggaccctgacctccaggagcttccaattttcttgaagagacaaatcagctggcatttcagtccagtgtgatctgctcttggtgagcacagacctagggagttggggcagcttcccagaagaactgcagtccaggctgagggcagagaaatgaggggaatggcgaggaattggggagcaggggggagctcagtagagagccaagggcgggaggtgagaagtccgtgttgggccaggagctaccctccggtggccacagccgaagtcgaggatgcctttggaactcatccccacttctctctttctgtatgtagccgtccaagaacaagtcacctccaagtgtagccggatcaaggcaagccccccatctagcaagcacttgatgccacccagaactgggcttcttcagaacaatctgagtccaggaatgatcccactcaccaggcaccagagctgcgagggcatgggagtgatctcaccaactctggggaagcggcaaggaattttcacctccagcccccagtgtcccatcctctcacactcaggccagactcccctgggcagacttgactctgtctgccagcatatgcagagccccaaggccaccccaccagaagtgcccctgcctgggttctgtcccagctccctgggcacccagtccttgagtccccaccagctcagacggcctagtgtgccaagaatgcccactgcgttcaacaatgctgcatgggtcacagcggcagcagctgtgaccacagcagtttcggggaaaacacccctcagccaagtggataatagcgttcagcagcactcaccttctggccaggcctgccttcagaggccatctgattgggaggcacaagtgcccgctgcgatgggaacacaagtgcccctggccaacaaccccagcttcagcctgctgggcagccagagcctcaggcagagcccggtacagggcccggtgcctgtagcaaacaccaccaagttcctccagcagggtatggccagctttagtcccctgagccccatacagggcatcgagccaccaagctatgtggctgctgctgccaccgctgctgctgcttctgccgttgctgccagccagttcccaggtccgttcgacagaacggatattccccctgagctgccacctgccgactttttgcgccagccccaacccccactaaatgatctgatttcgtcacctgactgcaatgaggtagatttcattgaagctctcttgaaaggctcctgtgtgagcccagatgaagactgggtgtgcaacttgaggctgatcgacgacattttggaacagcatgctgctgctcaaaatgccacagcccagaattctgggcaagtcacccaggatgctggggcactttaaatctgagcaggatgcccatagaaacccccatggtgacatcactctaggaagtggtgtcgatccatacccgcagttgtctcccgttacaatttgagtggtgttgtcagcccatgcttatccctctctctacctgtgacaaaatggaaagctggtgatttttcaagctacgtgtacatatttgaaaattttgtaaatggttttcctaaacattaatgacagaagtatttatacttcattttgtgactttgtaaataaagcgacggcttttgtttcagtagagttgtgtttactatgcattgttttgtgtttattatacaatgttacaaatatgcagaccgtgttgtttgctccagtgataccttgttaagctaggtggctgagtcgcttatggttttaatgcaatgagcaatgtggatatgaccaagagttgttgtgcaagttgacaaatgccaaatagaaaaccacttggccatttatttctatgttcactaaaaatcctattgccttgtgtgattcttaatctcttttgcgaacctttcagtctccgctagctctttcctaatgagctttacagcagaagctgttttatcgttaagtgccccacagagacactttaccaggaggctgggagagttctccagatttgggagaggcgcagagacagtgtgtgagccgagccctgtctcagcaatccacctggaggagctagagtatcctcctccctttaccattcagaccgagagaaaaagcccagcttgtgtgcaccctcgtggggttaaggcgagctgttcctggtttaaagcctttcagtatttgttttgatgtaaggctctgtggtttgggggggaacatctgtaaacattattagttgatttggggtttgtctttgatggtttctatctgcaattatcgtcatgtatatttaagtgtctgttatagaaaacccacacccactgtcctgtaaacttttctcagtgtccagactttctgtaatcacattttaattgccacctcgtatttcacctctacatttgaaatctggcgtctgtttcaagccagtgtgttttttcttcgttctgtaataaacagccaggagaaaagtgcctctatgtttttatttttcaagggagtattcagtacctacaaacccaagtcaggaagcctgctagtggctttggttctttcagaggctgctcgatgccttgtgtgtcagaaagaaagattcagcagttttgcatcatggcaaagaagcctgttattttggggctcagcccctcattttatagaggatgaaacagagggggatgggaggtcacaaagacaactgccccgggagcaggtgtgggggagacttgccctgagggtctagacgctctgcaccaccgtcctgtctcccttgctgaagaccacacatgcccttctttgaccagaccctgccacctgataggccaggacctggtaggcgggtacccaggtttcatggatggaaccacatctccccaaaagtggggaggtagctactgggatgcacgcctcccgccatgtgctataggagagcagctgaagcaacagttgggatcagatgtagtcacaattgaatgcatcatcacatttatccctctaagtggctgggagagttgatatcctcatccctaaggtacaaaatgttccaatttgatcagtggctttcaggagctgagaaaggcatgtgctctgaggcagagctgttatgtcccgcagagcctaaaaatgctctaagaacatgctccctgccaaaattctcaatggctgtgacaagggacaacgatcgaccaatgggggtggaagcagacctccgcagtccaggggccagagctaggacagaggggtcggagaaagagtcattttcccaacactccagctcttggccagtcctcacacagtcccctcctgcttcctgctgagagagatatcctcataggtctgggtaaagtccttcagtcagctttcattccctgtcaccaactttgtctctgttctccctgcccgtctcaggcagcactcctcaggaaacctctccaagagccagcctcactgcagcgcccactattgtccctctgcctcaagtgtcccatccatgccaggccccaggcaggctgcagctttccctcagggccacaccaaagcacttgggctcagctgtgctgtccccctccatcactgagctcaggggcagcaggggtggggtgccaggaggcccattcacccttctctggctctgtgttggacccacctgcccagccactgctgcttagaacctacccgctgggaaaatgaagccctcccggaggggccacctcaacctgagagcctcacggatcacagttgtccccactcagctctgccagccctcagagacccatagataaaagctgagcttggctcgcagagctggttccatcttccattcccagagggttcaacttcctaccccaaccacacagggaacctcaaggctgagccagtgtgggctgcagtgcagaccagcttcctggacacgtcctgccacctgaccccaggctggcctcactgcccctggcactcctgaccctatcctcattcctcctggcagtgcgtgttctgccattccgctttcccttagctgtcctctcactgtactgtcagcttctccttttccaggtgccccccaggggctttccacatgaccctgtcaccccacagcccatccagcaccaattccagctctctgccacccttcaaaggagtgacagtgccctgcttcacctcccactcacccctcaacccagagcaatctggctccagtcttgcctccttccccctaagtactctagtcacagttccaaattcctcctggtcataaagccaaatgaagcttcctggtcctcagcggacttgccacttcagcagtactggactctctcctcccagaaacctgtttccccttggctcctggagcccacactctgctggaatccttctgcctctctggcctgtagcctggccctctctcccaacctgaggtccattctctcctgctcctccacaagatgttgctccttccattacttcctccctctcaaccaaagctccttcattagctctttatcttctggtttcttcccctgggcagacgaatggattcaagagcctgtggcccagcagcccagcactccaggatctcagcacttcagcatcccagtaccctagcatctcaataccccagcaccccagcaccatagtattccagcaccccattgtccaagcatctcagcactccagcatcccagcaccccaacactccagcagcccagaatctcagcaccctagcactgcagcatctcaggaccccagcacttcagcatcccagcacactagtactccagcatctcggcaccccagcacctaggcatcccaacacccagcaccccagcacttaagcatcccaccactacagtatctcaacactccagcaccccagcaccatagtgttccagcaccccagcatcccaacaccccagcacttaagcatcccaacacctcggcatcccaacaccccagcactgcagcatctcagcaccttagcatcccagtgccctagcatctcaatgctccagcacaccagtactacagtattccagcaccccagcactccagcatctcagcactgcagcactgcagcactccagcatcccaaaatcccagcatcccaacaccccagcagaccagcagaccagcatctcagcaccgcagcatccaaggactatcccagcatcccagcaacccagcacctcagcatcccaacaccccagcatttcagcatggcaacaccccagtaccccagcacttcagcaccccagtatcccagcatctcagcgacccagtatcacaaaacctcagcatcctagcaccccagcaccccagcaccttagcaccttagcatcccagcatctcagcgcctcagcatcttgatattctggctgaggtcagcgtggtgtatctagtcagggtcctaactttcacttcgcagggaaatgctgctggactgggtctcatgttgggctgaagctctctagaccccttgaagacagcataaaagagcttggagacgctgggtgtcccccatggaagagttcactctcatcctgctttgacaacagccttctctggggtccctcacgggcccctctttcttactgcaagtttgtctctgagaagactgtgatgcagaagtcactcagctgcctgtggctcctgaagagctgaaggtggaggcctgtaggcctccctatgagaggcgcagaaaaaaccatgattgctagtggggaggtgctccctctacaacccactccataatctgcccccgcccagctctgaggccagccccaggggaaaatgccagatccccagggaggtgtgtgagacctcaggggctccctcctcccttacagcaggctcaggcccctgggggcctcagggccaaggtctgtgggtaagctactatctctcacttgtcctctagccacaaaagccagggagatctggcaatggacatgaggttctgaagaagcacatatgactggcttcctaatgcgtggttgttcagtgattcaataaacacgcatgggccaggcatggggaaatagacaaacatgatccccaacctctcccagagtgaactgggagggaggagtgttcatccctcaggattacaccagagaaacaaaccagcaggagatatatatggttttggggggtcaagaaagaggaaaaacctggcaaggcaagtccaaaatcataggacaggctgtcaggaagggcagcctggaacctctcaagcaggagctgatgctgcagtccacaggcagaatttcttcttcctcggggaaatctcagctttgttcttaaggcctttcaactgattggctgaggtctgccccttcccccacattctccaggataatcttccttacttaaagtcaactattaatcacagctacaaaatcccttcacagctacacatagatcagtgtttgattgacgaacagcccctacagcctagccaagttgacacataaaactaaccatcacagggggacaaatgatgtaaacacatcaacaaataaaacagtaacaagttaaggtctatggaaaaaacacagaaggggcagagagaaagaaagcaagaaggagagtcccagtttgctagggcttgtgggaagtggggagcagttctctttagctaggatatttgggaaaggcatatctgaaggagtgatatttgagcttagattaaaagatgggaaggagcaagccatgcaaagagctaggatgttccaagcagagacggaacagcaagtgcaaatgtcaggaggaatagaaggaggctggtgggtggggtccagtgagcaagaggagggcaggcaggagaggggatggggaggtgggcaggcccagaccacccagggccctggagactatcctgatccaacaagggaagccttgagtcacttcagtgtccatgtggagaatggacctcagactgaatgagggaggcagtaaggagggcctctacctccagggcttcgccctgtggactgcgcatagacatctccaactcagaaagtctgaaccaaactttccatagttcccccaagtctgggcatcctcctactcagtgaaaggcagccatcacacctccctgccctgctcccggatgccccaaatcctcttggtctccaagtccagaacctgagacttgtccttgatgtttgtctttccctcaccctttctgtattctgggaagatgggtttttttcccccagatgaatctgtaaaacttctgtgatcacaataaaaattctggcagtattattttctggaacatgacaaagtgattcaaaattatttatctggaagactacaaaacaagaatagccaggaaatttctaaaaagaaagaagaaggaggaggagaaagaaggaggaggaaaaggaggagaagaagaaaagaaaaagaaccaagaaagggttctagctctaccaaatattaaaacatatcatgaagctatttaaaacaatatggttgtggatactgaaaaagatgtgaataaagtggaaggaaaataaatagaaatgcacatggggattgagactgtgaaaaaggcagcatctcacatcagtgagggatgttcaacacctggtgttgggaaaactggctagtcatttaaaccaaacaactgggtcctctacctcactcctgacattaagatacatttagatgattcaaagagtaagacagaaaaaataacacgtgaaaacactatcagaaaacaacgtgggccaggtgtggtgggtcacgcctgtaatcccagcactttgggaggccgaggcagacagatcacctgaggtggggagttcaagaccagcctgaccaacatggtgaaatcctgtctctactaaaaatacaaaattagctgagcgtggtggcgcatgcctgtaatcccagctactcaggaggccgaggcaggagaatcacttgaacctgggaggcagaggttgtggtgagccgagatcacgccattgcactccagcctgggcaacaagagtgaaaatccatctaaaaaaaaaaaaaaaagccaaggtggatatttttatagtatcagggtagatcaagcttctccaatcatgacatgaaacccagaaaccataaaagaaaagaatgataaaattgcccacgtaaagtaaaaagcttgcacacagaaaaacaccatacaggttacaagatgagcagcaaaatcagagaaaaaacattgcaattcaggacacacagaggctattgttcctaatatttaaaaataaaagtagtggattgtctacaaaaagatgaagacaagaatttcagaaaaccaaatactgcatgttttcacttacaagtggaagctaaacactgagtacacgtgtacacaaagaatggaaccataggccaggcaccgtggctcacgcctgtaatcccagtactttgcgaggccgaagcgggcggatcacctgaggtgaggagttcgagaccatcctggccaacatggtgaaacccagtctctactaaaaatacaaaaattagccgggcgtggtggtgggtgcctgtaatcccagctactcgggaggctgcggcagtagaatcgcttgaaccctggaggtggaccttgcagtgagccgagatcgcaccactgcactccagcctgggcaacagagtgagactccatctcaaaaaaaaaaaaaaggaatagaacaatagacactggggcctacttgagggaggagggtgaggatcaaaaacctgcctatcaggtactatgcttattacctgggtggtgaaataatctgtacaccaaaccccagtgacatgcaatttaccgatgtaacaaacctgcccatgtacccgctgaacctaaaataaaagttggaaaaaaatatagaaattttctttgtaatagccaaaaactgcaaacagcccaggtgtctattagtagaatgcataaacaaactcgggcatgttcatacaatgtaaaactactcatcaataaaaagtgatacttctcagcaatgaaaagaaactagctactgataccagctacaacatggatggatttcaagtgctttatgatgagagcaagaagccagacacaaaagtgtctatatatatatacagtatatatacgtatatatacacatatatacagtatatatatacatatacatgtatatatatactgtatatatactgtatatatatacacagtatatatatacatatatacagtgtatatatactgtgtatatatacatgtatatatactgtgtatatatacatgtatatatactgtgtatatatacatgtatatatactgtgtatatatacatgtatatatatgtatactgtatatatactgtatatatatatacacatatatacagtatatatatacagtatatactgtatatatacagtatatacgtgtatatatacatatatacagtatatatgtaaatatacatatatacagtatatatgtaaatatacatatatacatgtatatatatacactatatatatacatatatagtgtatatatacatatatacatgtatatatttactatatgattccatttatataaagtgccaaaacagtcaaaaataatctatgtggaaaaaatcaacaaagggatcccccgggctgcaggaattcgatggcgcgccgtcgacccaacgcgttgggagctctccggatccaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacttagattcatcgagagggacacgtcgactactaaccttcttctctttcctacagctgagatcaccggcgaaggagggccaccatgcatggcatggaaaacacttcccatttacctgttgttgctgctgtctgttttcgtgattcagcaagtttcatctcaagatttatcaagctgtgcagggagatgtggggaagggtattctagagatgccacctgcaactgtgattataactgtcaacactacatggagtgctgccctgatttcaagagagtctgcactgcggagctttcctgtaaaggccgctgctttgagtccttcgagagagggagggagtgtgactgcgacgcccaatgtaagaagtatgacaagtgctgtcccgattatgagagtttctgtgcagaagtgcataatcccacatcaccaccatcttcaaagaaagcacctccaccttcaggagcatctcaaaccatcaaatcaacaaccaaacgttcacccaaaccaccaaacaagaagaagactaagaaagttatagaatcagaggaaataacagaagaacattctgtttctgaaaatcaagagtcctcctcctcctcctcctcttcctcttcttcttcaacaattcggaaaatcaagtcttccaaaaattcagctgctaatagagaattacagaagaaactcaaagtaaaagataacaagaagaacagaactaaaaagaaacctacccccaaaccaccagttgtagatgaagctggaagtggattggacaatggtgacttcaaggtcacaactcctgacacgtctaccacccaacacaataaagtcagcacatctcccaagatcacaacagcaaaaccaataaatcccagacccagtcttccacctaattctgatacatctaaagagacgtctttgacagtgaataaagagacaacagttgaaactaaagaaactactacaacaaataaacagacttcaactgatggaaaagagaagactacttccgctaaagagacacaaagtatagagaaaacatctgctaaagatttagcacccacatctaaagtgctggctaaacctacacccaaagctgaaactacaaccaaaggccctgctctcaccactcccaaggagcccacgcccaccactcccaaggagcctgcatctaccacacccaaagagcccacacctaccaccatcaagtctgcacccaccacccccaaggagcctgcacccaccaccaccaagtctgcacccaccactcccaaggagcctgcacccaccaccaccaaggagcctgcacccaccactcccaaggagcctgcacccaccaccaccaaggagcctgcacccaccaccaccaagtctgcacccaccactcccaaggagcctgcacccaccacccccaagaagcctgccccaactacccccaaggagcctgcacccaccactcccaaggagcctacacccaccactcccaaggagcctgcacccaccaccaaggagcctgcacccaccactcccaaagagcctgcacccactgcccccaagaagcctgccccaactacccccaaggagcctgcacccaccactcccaaggagcctgcacccaccaccaccaaggagccttcacccaccactcccaaggagcctgcacccaccaccaccaagtctgcacccaccactaccaaggagcctgcacccaccactaccaagtctgcacccaccactcccaaggagccttcacccaccaccaccaaggagcctgcacccaccactcccaaggagcctgcacccaccacccccaagaagcctgccccaactacccccaaggagcctgcacccaccactcccaaggaacctgcacccaccaccaccaagaagcctgcacccaccactcccaaagagcctgccccaactacccccaaggagactgcacccaccacccccaagaagctcacgcccaccacccccgagaagctcgcacccaccacccctgagaagcccgcacccaccacccctgaggagctcgcacccaccacccctgaggagcccacacccaccacccctgaggagcctgctcccaccactcccaaggcagcggctcccaacacccctaaggagcctgctccaactacccctaaggagcctgctccaactacccctaaggagcctgctccaactacccctaaggagactgctccaactacccctaaagggactgctccaactaccctcaaggaacctgcacccactactcccaagaagcctgcccccaaggagcttgcacccaccaccaccaaggagcccacatccaccacctgtgacaagcccgctccaactacccctaaggggactgctccaactacccctaaggagcctgctccaactacccctaaggagcctgctccaactacccctaaggggactgctccaactaccctcaaggaacctgcacccactactcccaagaagcctgcccccaaggagcttgcacccaccaccaccaaggggcccacatccaccacctctgacaagcctgctccaactacacctaaggagactgctccaactacccccaaggagcctgcacccactacccccaagaagcctgctccaactactcctgagacacctcctccaaccacttcagaggtctctactccaactaccaccaaggagcctaccactatccacaaaagccctgatgaatcaactcctgagctttctgcagaacccacaccaaaagctcttgaaaacagtcccaaggaacctggtgtacctacaactaagactcctgcagcgactaaacctgaaatgactacaacagctaaagacaagacaacagaaagagacttacgtactacacctgaaactacaactgctgcacctaagatgacaaaagagacagcaactacaacagaaaaaactaccgaatccaaaataacagctacaaccacacaagtaacatctaccacaactcaagataccacaccattcaaaattactactcttaaaacaactactcttgcacccaaagtaactacaacaaaaaagacaattactaccactgagattatgaacaaacctgaagaaacagctaaaccaaaagacagagctactaattctaaagcgacaactcctaaacctcaaaagccaaccaaagcacccaaaaaacccacttctaccaaaaagccaaaaacaatgcctagagtgagaaaaccaaagacgacaccaactccccgcaagatgacatcaacaatgccagaattgaaccctacctcaagaatagcagaagccatgctccaaaccaccaccagacctaaccaaactccaaactccaaactagttgaagtaaatccaaagagtgaagatgcaggtggtgctgaaggagaaacacctcatatgcttctcaggccccatgtgttcatgcctgaagttactcccgacatggattacttaccgagagtacccaatcaaggcattatcatcaatcccatgctttccgatgagaccaatatatgcaatggtaagccagtagatggactgactactttgcgcaatgggacattagttgcattccgaggtcattatttctggatgctaagtccattcagtccaccatctccagctcgcagaattactgaagtttggggtattccttcccccattgatactgtttttactaggtgcaactgtgaaggaaaaactttcttctttaaggattctcagtactggcgttttaccaatgatataaaagatgcagggtaccccaaaccaattttcaaaggatttggaggactaactggacaaatagtggcagcgctttcaacagctaaatataagaactggcctgaatctgtgtattttttcaagagaggtggcagcattcagcagtatatttataaacaggaacctgtacagaagtgccctggaagaaggcctgctctaaattatccagtgtatggagaaacgacacaggttaggagacgtcgctttgaacgtgctataggaccttctcaaacacacaccatcagaattcaatattcacctgccagactggcttatcaagacaaaggtgtccttcataatgaagttaaagtgagtatactgtggagaggacttccaaatgtggttacctcagctatatcactgcccaacatcagaaaacctgacggctatgattactatgccttttctaaagatcaatactataacattgatgtgcctagtagaacagcaagagcaattactactcgttctgggcagaccttatccaaagtctggtacaactgtccttagaaaaaaaaaaaaaaaaaggccacatgtgctcgagctgcaggtcgcggccctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatcatttggggtaccgaattcctcgagtctagaggagcatgcgacgtcggcgcgcctaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttt

SEQ ID NO: 9 - DNA, 31815 bp, synthetic construct
  misc_feature 1..31815: note = HDV with murine PRG4
  source 1..31815: mol_type = other DNA
aaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaactggacatttgttctaatctttgaagagtatgaaaaattcctattttaaaggtaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcatttcaattgtgtgacacaggcagactgtggatcaaaagtggtttctggtgcgacttactctcttgagtatacctgcagtcccctttcttaagtgtgttaaaaaaaaagggggatttcttcaattcgccaatactctagctctccatgtgctttctaggaaacaagtgttaacccaccttatttgtcaaacctagctccaaaggacttttgactccccacaaaccgatgtagctcaagagagggtatctgtcaccagtatgtatagtgaaaaaagtatcccaagtcccaacagcaattcctaaaaggagtttatttaaaaaaccacacacacctgtaaaataagtatatatcctccaaggtgactagttttaaaaaaacagtattggctttgatgtaaagtactagtgaatatgttagaaaaatctcactgtaaccaagtgaaatgaaagcaagtatggtttgcagagattcaaagaaaatataagaaaacctactgttgccactaaaaagaatcatatattaaatatactcacacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtatcacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaattttctggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacacactaatattaaaagtgtccaataacatttaaaactatactcatacgttaaaatataaatgtatatatgtacttttgcatatagtatacatgcataggccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatgaggaataaacaccctttccaaatcctcagcataatgattaggtatgcaaaataaatcaaggtcataacctggttcatcatcactaatctgaaaaagaaatatagctgtttcaatgagagcattacaggatacaaacatttgattggattaagatgttaaaaaataaccttagtctatcagagaaatttaggtgtaagatgatattagtaactgttaactttgtaggtatgataatgaattatgtaagaaaacaacaggccgggcgggttggttcacacgtgtaatcccagcactttgggaggctgaggcaggcagactgcctgagctcaggagttcgagaccagcctgggcaacacggtgaaatcccgtctctactaaaaatacaaaaaaattagccgggtgtggtgacacatgcctgtagtcccagctacttgggaggctgaggcaggagaatcacttgaacctgggaggtgaaggttgcagtgagccaagatggcaccacttcactccagcctgggaaacagagcaagactctgtctctgagctgagatggcaccacttcactccagcctgggaaacagagcaagactctgtctcaaaaaaaacaaaacacacaaacaaaaaaacaggctgggcgcggtggctcacgcctgtaatcccagcactttgggaggccgaggcgggtggatcacctgaggtcaggagttccagaccagccttgtcaacatggtgaaacctccccccgccgtctctactaaaaatacaaaaattagccaggcgtggtggcaggagcctgtaatcccagctacttgggaggctgaggcaggagaatcgcttgtacccagaaggcagaggttgcactgagctgagatggcaccattgcactccagcctgggggacaagagcgagatttcgtctttaaaaaacaaaaacaaaacaaaaaaccatgtaactatatgtcttagtcatcttagtcaagaatgtagaagtaaagtgataagatatggaatttcctttaggtcacaaagagaaaaagaaaaattttaaagagctaagacaaacgcagcaaaatctttatatttaataatattctaaacatgggtgatgaacatacgggtattcattatactattctctccacttttgagtatgtttgaaaatttagtaaaacaagttttaacacactgtagtctaacaagataaaatatcacactgaacaggaaaaactggcatggtgtggtggctcacacttgtaatcccagtgctttgggaggctgagacaggagagttgcttgaggccaggagttcaagaccgacatggggaatgtagcaagaccccgtccctacaaaaaactttgtaaaaatttgccaggtatggtggtgcatacctgtagtcccagctactcgggaggcggaggcagaaggaatcacttgagcccaggagtttgaggctgcagtgagctacgatcataccacagcactccagcgtggacaacagagtaagaccctatctcaaaaacaaaacaaaacaaaacaaacaaaaaaaaccacaagaaaaactgctggctgatgcagcggctcatgcctgtaatcccagtattttgggaggcccaggtgggcgtatcacctgaggtcaggagttagagaccagcctggccaacatggtgaaaccccatctctactaaaaatacaaaattagccaggcatgtggcacgcgcctgtagtcccagttactgggaggctgaagcaggaggatcacctgagcccgggaggtggaggttgcagtgagccgagatcacaccactgcactccagcctgggtgacacagcaataccctacctcaaaataaaaaagaaaaagaaaagaaaagttgctgtccccgctaccccaatcccaaatccaaacagcctctctcatctcacagtaagggggaaaaatcacccaaaaaagctaagtgatcttttgaaaacccaaactcttagaagtctaagattattatagtcaactcatgaagtgtcatcataaaagatactctaatattatttaagtagaaccacatattggttgtcttggtatgtctagcccctggcatacaaaatatttaataacactgatatggtacctgtgatgtgaaaatgtactatgagtacagctttataaatactatatatgtacctatatacagaaaaaaatacaacaaaatcataaaagcacttatctttgaaagaggagttacagcaattttatttagttctttattgctttgctatatattctaaatttttttcaatgaatatatatcacttttaaaaaaattcaatggtctttcttataaattatctttggcagcatgcgtttttatatatacatataaaatgtatgggaaatttttaaaggatacattaaattaaagcaaaatatacaaacaaaaaatcagaatacaaaaagataaaaagattgggaagggagggagggagtaaggaggaagggtgggtgggtatagagaaatataccaaataatggtaagaagtggggtcttgacactttctacactttttttaaataaaaaaaatttttttctctctcttttttttttttagagacgaagtctcgctatgttgcccaggctggtcttgaactcctgggatcaagagatcctcctgcctcagcctcccaaggtgcttggattacaggtgtgagccaccacgcctggtcactttctacactttaatatatatattttttcattttcaatgtcatttttattagttaatttataatacccattcaccattatattcaaagtctatttgaagaaataaaccagaaagaatgaaatactctagctcacatgctattcaatactaaattacctttcaaatcacattcaagaagctgatgatttaagctttggcggtttccaataaatattggtcaaaccataattaaatctcaatatatcagttagtacctattgagcatctccttttacaacctaagcattgtattaggtgcttaaatacaagcagcttgacttttaatacatttaaaaatacatatttaagacttaaaatcttatttatggaattcagttatattttgaggtttccagtgctgagaaatttgaggtttgtgctgtctttcagtccccaaagctcagttctgagttctcagactttggtggaacttcatgtattgtcaggttggcccgtaatacctgtgggacaacttcagcccctgtgcacatggccaggaggctggttgcaaacattttcaggtaggtggaccaggacatgcccctggtcatggccaggtggaggcatagtgctatacagcaggcagaagtcaatattgatttgtttttaaagaaacatgtactactttcataagcagaaaaaatttctattcttgggggaaaagattatgccagatcctctaggattaaatgctgatgcatctgctaaaccttcacatatcagaacatatttactatagaaagaatgaaaatgggacatttgtgtgtcacctatgtgaacattccaaaaatattttacaacaactaagtattttataaattttatgaactgaaatttagttcaagttctaggaaaatacaaaccttgctagatattataaaaatgatacaatatatattcatttcaggctcatcagaatatatctgttatcacttgacaagaatgaaaatgcaccattttgtagtgctttaaaatcaggaagatccagagtactaaaaatgacttcttccttgaagcttactcaccaacttcctcccagttactcactgcttctgccacaagcataaactaggacccagccagaactcccttgaaatatacacttgcaacgattactgcatctatcaaaatggttcagtgcctggctacaggttctgcagatcgactaagaatttgaaaagtcttgtttatttcaaaggaagcccatgtgaattctgcccagagttcatcccagatatgcagtctaagaatacagacagatcagcagagatgtattctaaaacaggaattctggcaatataacaaattgatttccaatcaaaacagatttacataccatacttatgtcaagaagttgttttgttttattgcatcctagattttatttttttgatttatggtttactttaagcataaaaaatttgtcaatacaactcttcccaaaaggcataaacaaaaattcataaaacttgcatcacttgagatacttcaggtatgaattcacaactttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtcgtcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcatttaaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactcctgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagctatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaagcttaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaaggtacctgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgagagctctggaggggcctagaagtggtaaatgaatgaaaacgtggttactctccagatctgcctttcccaaatatggccattcttggctgaatcagaaatcaaaggacaggttattaattactagctctaagttacttaccatttgctgagacagttcagaaatctgactgcatctcctcagagatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctataatcgattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttaaaaaggaaaaaaaaaaaaaagacatgttggccagatgcggtggctcacacctgtaatcccagcactttgggaggctgaggtgggcggatcacccctggcctgcccaacatggtgaaaccccgtctctactaaaaacacaaaaattagctgggcgtggtggcgggcgcctgtaattccagctactcgggaggctgaggcaggagaattgctagaacccaggaggcagagattgcagtgagctgagattgcaccattgcactacagtctgggcaacaaaagtgaaactccatcttaaaaaaaaaaagacaatgttcgtgggtccaaacaagacttaatggaagtgagtctaaaaatgagctatgtgggccaggcgtagtggctcccacctgtaatcccagcactttgggaggccgaagcaggcagatcatgaggtcaggagatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagatttgcatcactgcactcctgcctggtgacagagcaagactccatctcaaaaaaaacaaacaaaaataaaagataaaaatgagctatgtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatgatccgccatctccttggataatttgtgttaactcaggataacagaaaaccaaaattatgagttactgatgcaatattcctaaatgtaaaaataattaaagctaatagtagattcatcttccaatttcatatcagtcttacaaataaactacatatataacttgcttgccttcccttctgagggataaagctgttagaagaattaaaatcagcattcttgactattcaaccaagggagggataaattattactcattctagggacatgggctcataactactacatgtgtaaggacatgaatttacccaatattacaatttttccttttattagtgtgtacagtggaagaatagacatgttcactctggacaaaaaaaaaattatacttatcagttatcagaagcacaatgctgaagacagtagttccataacaatttgaagtatgtgatcgaactagtagattatcttagtagtagtgaattattgtaaatgttagtaatttggcagccactgggcagaaaaataagaattgaggctcaatattgatattaatggtggtgattgacacataaattttatcaagtctacacaatataaaattacagaaaggtagaagagtataccagtacaacttcaacatatcttcactacaagggagtaaaatgacatggcctagttactatctaatgaactgcagaaaactaaaagaaaactccaaggcaactcttctctgctgatctggttggtccttttcctaccttttgcaatacccagatacaaacaatggatagaaaacaaagtagacttgtagtatgcaggtcacagtgctaaattcacagaaagaaacccctgaactgaactgctctatttcctggtggtcacaaagagtaattctggtttacacctacagattgatgtcaatctacaccctgttgataacagtgtggccaaggacaaaaaaaaggtgctccgttttaccaattctgtaaaaaattattggcagggtaagctcggctagggcaggattacatttctaggactaccatccccgaaatttagaagatattatatccacataaagcatatctttcacattaatttgcaaaaatctaaaagctttttcttagctcaagtgtgtccaagtttaccctggcagtttaaaacgatagttacaagcagcatgggttgtatcagacacatttgagggccaatttcatgtaagtgatattgggcaagttacttcaactatctgtgcctccaaggtcatactagtgtttatttacctaaagggtacctgttatgtaactttagggtgtttacattagataatgcctgcaaaatatttacttcaacgcctaaaacatagttaagtattcaataaatacctactattgtcactactaacttaaaagtttagagattaagagcagaatctggggtgagacaaacttaggttcaaatcctagtattgttgggtaatcttgggcaagttacttaacctctctgatttgtgtaatttaaaaaattagttaatatacataacagggcttagaagagtatctagcacatagcaccatttaagcatttgttattgctaacatgcaaacaatttaagggaaagaaattttttaaaaaggaagagggatttgcaaactaaaaacaatgagtatcttatgttcaaagaaaactaacaaacagccagctctagcaataattaaattcactatatactggggcaggcatcacaccccaaagctaaaagcgtctacctaggccaggcacggtggctcatgcctgtaatcccagcactttgggaagcagaggcgggcagatcgcttgagctcaggagttcaagaccagcctggacaacatggcaaaacaccatctctacaaaaaatacaaatattaggccgggcgcagtggctcacgcctgtaatcccagcactttgggaggccaaggcgggtggatcacctgagatcaggagttcgagagtagcctggccaacatggtgaaacctcgtctctattaaaaatacaaaaaattagccaggcatggtggcaggcgcctgtaatcccagctactcagggggatgaggtaggagaatcgcttgaacccgggaggcagaggttgcactgagccgagatcatgccactgtactccagcccgggcaacaagagcgaaactccatctcaaaaaataaataaataaataaataaaataaagtacaaatattagccagggatggtggtgcgcacctgtagtcccagctacttgggaggctgaagtgggagaatcccctgagcctggggagaatcacccgagcccgggaagtcgaggctgcagtgagcagtgattgtgccactgcactccatcctaggtgacagagtgagaccctgtctcaaaaaaaagaaattggcagaattaagtaagttgatgtttagagatgaaaaatcaacattttttcctcagcaactgaataaaaacaacagccactaccatttttttgagtacctatttgtagcctattttttaactggtattactcgagagagagagagctaggttcgagacagagctccttctcttaataactgtatgacctagggtatgtctgttagcctctctgaggcttcaaaggttcctcatctgtaaaatggtaataatcataccattgctacagggctgttttgaagactaattaggactatgtaagtaaacatgatgatggctattattactgttccccgccaggggccatgcaagggttgctgattcacatagactgtcttataatcctctcaataactccaagaggtagccagcacctcagatatacataaaatgacttaagcccagagaggtgaagtaagttgcccacagccacacaactagtaaatagcccaaacaagctggattcccagttagactccgttaatagcactgctctttaccttaagtcattacaatgcctaatatgaaatagaatcgcttctttcttagggttcaagtggttaattatttaatgtattcattcaacaaaccatcatcgaggacctcttacaagccaagtactgtgctaagtgctagagttacggcggtgattcctgcccttaaaaagttttagtgggagaaacaacaggtaaccaggtcattgccaaaacaacaaaaataatcataataaagcaggctaaagcatatttaactggccggggttttgactattttagcaagcatgatcagaacggttgaggagggaggccagcagcttggccggttcaacaaacaagaaaaaaccagtgagggtggagctaagataccagaggctgattacggttaagaatgttcttgaaggtaaggaccagattctcattttctatatcctggggcatcggtcagcatggaatctggattctagcacatgtgaatttcggcttgaaatgacctaatgccttttccctagttccttcgtgtgtcaaatacgcatggttaccgctaccagagctgtagtggggcttcaatgaggccatgagcatctccataaagatgaactacagtgtgtgcaaaactaaaggcaaaacctggtccccacacgccctcccaggtggtcgctttccgtgccgaggcccctccagaggtgccccgagaacctcaccatcgcaccccaaacttccagggaagggcctctcccgagaaagcccccacgcccccaccccgcgccatcattcccgaatctgccctcggcccctccccgcagcacgctcgcaggcggcacatgtcaaccaaaacgccatttccaccttctcttcccacacgcagtcctcttttcccagggctcccccgaggagggacccaccccaaaccccgccattccgtcctccctgccgccctcgcgtgacgtaaagccgaacccgggaaactggccgcccccgcctgcggggttccctgggcccggccgctctagaactagtggatcccaattgaaggcctggtctaaatgactccaaaatcaccacttaattcaagagactgatttccctgagtcaggccccttaaagcagctatttcaatgggacagggaaacaaccctaggatctggattagaatcacttgggggctgccacacccccagggctctgatcctgcccttctcccacacgcacattcacatactgctgcagtgaccttccatttctaatgggttcctgggccatctgtcaggtatagggaatggaaaaggggttggggaggctctgcttcagaaagtttgtgtcaggggctcccagagcctccacagatagatagcaggggtccccaccctaccatggcagctataaatgtgatcaacatttattggcctaggatacagcagttagcaaaatgcctgatgtagttcccactccgtggaggttgcaggctagccaagaagtcatgagttcagcaacccttacgcaccagtgggatgagattggaccaggccgagggtagtcttgggaacactcagcatttgtctgagggccagaagaggctgcttgccctcagacaggaggtcagcatctttattgtagcccatgacacctctacaccattgctcttctggtcttatggaagacatctttgggcctgataacagcggagtctgtgtcccacttgtccaggctggagtgccacatcaggcacactccagttgcagggacagcacagacaagtttcaggaaggctggtggcctccaggaggttaaccttataaggccagattgtaacctagttgaaaaacatacacatgccatgataataaaagaacctaggcaccattacaagagaaaaaatcatttttgtagatacgagcatggattcttgggtgggtcagacacactgggcttgtgctctgactgcactgtctcccctacctgaccttgggtaaaccataagactgctgcatgactcagtgtccaccccaaaaaagtaccggtagatattggccacagtagatatcagctagagtggactctcatgacaatgaggggagatgtattccccatcttaggcacctgggactctaccttccatcttctgctccgtgtctctccatccccaggctcttcagaactcagggagtccagaatgtcagctcccagatttcagccttcagaaaggaaacccattaccgttcagttgaacaaatgttgtctgagccccagatctgggctcagaggccatctaggctatgagacaagaggggaacaaagcaccgtctgcactcactcaccacactcacttgctgtcccaggtcacatccatcgggtagagaatctaagaggctgagctagctcccgccaccagcccagcccaccccacctggccccttccttccttctacaaaatatgcaccacctgtcaaagggtgggcagtgccaggcctgcatacagagcactgagtgtaaaagcagacatggaccctgacctccaggagcttccaattttcttgaagagacaaatcagctggcatttcagtccagtgtgatctgctcttggtgagcacagacctagggagttggggcagcttcccagaagaactgcagtccaggctgagggcagagaaatgaggggaatggcgaggaattggggagcaggggggagctcagtagagagccaagggcgggaggtgagaagtccgtgttgggccaggagctaccctccggtggccacagccgaagtcgaggatgcctttggaactcatccccacttctctctttctgtatgtagccgtccaagaacaagtcacctccaagtgtagccggatcaaggcaagccccccatctagcaagcacttgatgccacccagaactgggcttcttcagaacaatctgagtccaggaatgatcccactcaccaggcaccagagctgcgagggcatgggagtgatctcaccaactctggggaagcggcaaggaattttcacctccagcccccagtgtcccatcctctcacactcaggccagactcccctgggcagacttgactctgtctgccagcatatgcagagccccaaggccaccccaccagaagtgcccctgcctgggttctgtcccagctccctgggcacccagtccttgagtccccaccagctcagacggcctagtgtgccaagaatgcccactgcgttcaacaatgctgcatgggtcacagcggcagcagctgtgaccacagcagtttcggggaaaacacccctcagccaagtggataatagcgttcagcagcactcaccttctggccaggcctgccttcagaggccatctgattgggaggcacaagtgcccgctgcgatgggaacacaagtgcccctggccaacaaccccagcttcagcctgctgggcagccagagcctcaggcagagcccggtacagggcccggtgcctgtagcaaacaccaccaagttcctccagcagggtatggccagctttagtcccctgagccccatacagggcatcgagccaccaagctatgtggctgctgctgccaccgctgctgctgcttctgccgttgctgccagccagttcccaggtccgttcgacagaacggatattccccctgagctgccacctgccgactttttgcgccagccccaacccccactaaatgatctgatttcgtcacctgactgcaatgaggtagatttcattgaagctctcttgaaaggctcctgtgtgagcccagatgaagactgggtgtgcaacttgaggctgatcgacgacattttggaacagcatgctgctgctcaaaatgccacagcccagaattctgggcaagtcacccaggatgctggggcactttaaatctgagcaggatgcccatagaaacccccatggtgacatcactctaggaagtggtgtcgatccatacccgcagttgtctcccgttacaatttgagtggtgttgtcagcccatgcttatccctctctctacctgtgacaaaatggaaagctggtgatttttcaagctacgtgtacatatttgaaaattttgtaaatggttttcctaaacattaatgacagaagtatttatacttcattttgtgactttgtaaataaagcgacggcttttgtttcagtagagttgtgtttactatgcattgttttgtgtttattatacaatgttacaaatatgcagaccgtgttgtttgctccagtgataccttgttaagctaggtggctgagtcgcttatggttttaatgcaatgagcaatgtggatatgaccaagagttgttgtgcaagttgacaaatgccaaatagaaaaccacttggccatttatttctatgttcactaaaaatcctattgccttgtgtgattcttaatctcttttgcgaacctttcagtctccgctagctctttcctaatgagctttacagcagaagctgttttatcgttaagtgccccacagagacactttaccaggaggctgggagagttctccagatttgggagaggcgcagagacagtgtgtgagccgagccctgtctcagcaatccacctggaggagctagagtatcctcctccctttaccattcagaccgagagaaaaagcccagcttgtgtgcaccctcgtggggttaaggcgagctgttcctggtttaaagcctttcagtatttgttttgatgtaaggctctgtggtttgggggggaacatctgtaaacattattagttgatttggggtttgtctttgatggtttctatctgcaattatcgtcatgtatatttaagtgtctgttatagaaaacccacacccactgtcctgtaaacttttctcagtgtccagactttctgtaatcacattttaattgccacctcgtatttcacctctacatttgaaatctggcgtctgtttcaagccagtgtgttttttcttcgttctgtaataaacagccaggagaaaagtgcctctatgtttttatttttcaagggagtattcagtacctacaaacccaagtcaggaagcctgctagtggctttggttctttcagaggctgctcgatgccttgtgtgtcagaaagaaagattcagcagttttgcatcatggcaaagaagcctgttattttggggctcagcccctcattttatagaggatgaaacagagggggatgggaggtcacaaagacaactgccccgggagcaggtgtgggggagacttgccctgagggtctagacgctctgcaccaccgtcctgtctcccttgctgaagaccacacatgcccttctttgaccagaccctgccacctgataggccaggacctggtaggcgggtacccaggtttcatggatggaaccacatctccccaaaagtggggaggtagctactgggatgcacgcctcccgccatgtgctataggagagcagctgaagcaacagttgggatcagatgtagtcacaattgaatgcatcatcacatttatccctctaagtggctgggagagttgatatcctcatccctaaggtacaaaatgttccaatttgatcagtggctttcaggagctgagaaaggcatgtgctctgaggcagagctgttatgtcccgcagagcctaaaaatgctctaagaacatgctccctgccaaaattctcaatggctgtgacaagggacaacgatcgaccaatgggggtggaagcagacctccgcagtccaggggccagagctaggacagaggggtcggagaaagagtcattttcccaacactccagctcttggccagtcctcacacagtcccctcctgcttcctgctgagagagatatcctcataggtctgggtaaagtccttcagtcagctttcattccctgtcaccaactttgtctctgttctccctgcccgtctcaggcagcactcctcaggaaacctctccaagagccagcctcactgcagcgcccactattgtccctctgcctcaagtgtcccatccatgccaggccccaggcaggctgcagctttccctcagggccacaccaaagcacttgggctcagctgtgctgtccccctccatcactgagctcaggggcagcaggggtggggtgccaggaggcccattcacccttctctggctctgtgttggacccacctgcccagccactgctgcttagaacctacccgctgggaaaatgaagccctcccggaggggccacctcaacctgagagcctcacggatcacagttgtccccactcagctctgccagccctcagagacccatagataaaagctgagcttggctcgcagagctggttccatcttccattcccagagggttcaacttcctaccccaaccacacagggaacctcaaggctgagccagtgtgggctgcagtgcagaccagcttcctggacacgtcctgccacctgaccccaggctggcctcactgcccctggcactcctgaccctatcctcattcctcctggcagtgcgtgttctgccattccgctttcccttagctgtcctctcactgtactgtcagcttctccttttccaggtgccccccaggggctttccacatgaccctgtcaccccacagcccatccagcaccaattccagctctctgccacccttcaaaggagtgacagtgccctgcttcacctcccactcacccctcaacccagagcaatctggctccagtcttgcctccttccccctaagtactctagtcacagttccaaattcctcctggtcataaagccaaatgaagcttcctggtcctcagcggacttgccacttcagcagtactggactctctcctcccagaaacctgtttccccttggctcctggagcccacactctgctggaatccttctgcctctctggcctgtagcctggccctctctcccaacctgaggtccattctctcctgctcctccacaagatgttgctccttccattacttcctccctctcaaccaaagctccttcattagctctttatcttctggtttcttcccctgggcagacgaatggattcaagagcctgtggcccagcagcccagcactccaggatctcagcacttcagcatcccagtaccctagcatctcaataccccagcaccccagcaccatagtattccagcaccccattgtccaagcatctcagcactccagcatcccagcaccccaacactccagcagcccagaatctcagcaccctagcactgcagcatctcaggaccccagcacttcagcatcccagcacactagtactccagcatctcggcaccccagcacctaggcatcccaacacccagcaccccagcacttaagcatcccaccactacagtatctcaacactccagcaccccagcaccatagtgttccagcaccccagcatcccaacaccccagcacttaagcatcccaacacctcggcatcccaacaccccagcactgcagcatctcagcaccttagcatcccagtgccctagcatctcaatgctccagcacaccagtactacagtattccagcaccccagcactccagcatctcagcactgcagcactgcagcactccagcatcccaaaatcccagcatcccaacaccccagcagaccagcagaccagcatctcagcaccgcagcatccaaggactatcccagcatcccagcaacccagcacctcagcatcccaacaccccagcatttcagcatggcaacaccccagtaccccagcacttcagcaccccagtatcccagcatctcagcgacccagtatcacaaaacctcagcatcctagcaccccagcaccccagcaccttagcaccttagcatcccagcatctcagcgcctcagcatcttgatattctggctgaggtcagcgtggtgtatctagtcagggtcctaactttcacttcgcagggaaatgctgctggactgggtctcatgttgggctgaagctctctagaccccttgaagacagcataaaagagcttggagacgctgggtgtcccccatggaagagttcactctcatcctgctttgacaacagccttctctggggtccctcacgggcccctctttcttactgcaagtttgtctctgagaagactgtgatgcagaagtcactcagctgcctgtggctcctgaagagctgaaggtggaggcctgtaggcctccctatgagaggcgcagaaaaaaccatgattgctagtggggaggtgctccctctacaacccactccataatctgcccccgcccagctctgaggccagccccaggggaaaatgccagatccccagggaggtgtgtgagacctcaggggctccctcctcccttacagcaggctcaggcccctgggggcctcagggccaaggtctgtgggtaagctactatctctcacttgtcctctagccacaaaagccagggagatctggcaatggacatgaggttctgaagaagcacatatgactggcttcctaatgcgtggttgttcagtgattcaataaacacgcatgggccaggcatggggaaatagacaaacatgatccccaacctctcccagagtgaactgggagggaggagtgttcatccctcaggattacaccagagaaacaaaccagcaggagatatatatggttttggggggtcaagaaagaggaaaaacctggcaaggcaagtccaaaatcataggacaggctgtcaggaagggcagcctggaacctctcaagcaggagctgatgctgcagtccacaggcagaatttcttcttcctcggggaaatctcagctttgttcttaaggcctttcaactgattggctgaggtctgccccttcccccacattctccaggataatcttccttacttaaagtcaactattaatcacagctacaaaatcccttcacagctacacatagatcagtgtttgattgacgaacagcccctacagcctagccaagttgacacataaaactaaccatcacagggggacaaatgatgtaaacacatcaacaaataaaacagtaacaagttaaggtctatggaaaaaacacagaaggggcagagagaaagaaagcaagaaggagagtcccagtttgctagggcttgtgggaagtggggagcagttctctttagctaggatatttgggaaaggcatatctgaaggagtgatatttgagcttagattaaaagatgggaaggagcaagccatgcaaagagctaggatgttccaagcagagacggaacagcaagtgcaaatgtcaggaggaatagaaggaggctggtgggtggggtccagtgagcaagaggagggcaggcaggagaggggatggggaggtgggcaggcccagaccacccagggccctggagactatcctgatccaacaagggaagccttgagtcacttcagtgtccatgtggagaatggacctcagactgaatgagggaggcagtaaggagggcctctacctccagggcttcgccctgtggactgcgcatagacatctccaactcagaaagtctgaaccaaactttccatagttcccccaagtctgggcatcctcctactcagtgaaaggcagccatcacacctccctgccctgctcccggatgccccaaatcctcttggtctccaagtccagaacctgagacttgtccttgatgtttgtctttccctcaccctttctgtattctgggaagatgggtttttttcccccagatgaatctgtaaaacttctgtgatcacaataaaaattctggcagtattattttctggaacatgacaaagtgattcaaaattatttatctggaagactacaaaacaagaatagccaggaaatttctaaaaagaaagaagaaggaggaggagaaagaaggaggaggaaaaggaggagaagaagaaaagaaaaagaaccaagaaagggttctagctctaccaaatattaaaacatatcatgaagctatttaaaacaatatggttgtggatactgaaaaagatgtgaataaagtggaaggaaaataaatagaaatgcacatggggattgagactgtgaaaaaggcagcatctcacatcagtgagggatgttcaacacctggtgttgggaaaactggctagtcatttaaaccaaacaactgggtcctctacctcactcctgacattaagatacatttagatgattcaaagagtaagacagaaaaaataacacgtgaaaacactatcagaaaacaacgtgggccaggtgtggtgggtcacgcctgtaatcccagcactttgggaggccgaggcagacagatcacctgaggtggggagttcaagaccagcctgaccaacatggtgaaatcctgtctctactaaaaatacaaaattagctgagcgtggtggcgcatgcctgtaatcccagctactcaggaggccgaggcaggagaatcacttgaacctgggaggcagaggttgtggtgagccgagatcacgccattgcactccagcctgggcaacaagagtgaaaatccatctaaaaaaaaaaaaaaaagccaaggtggatatttttatagtatcagggtagatcaagcttctccaatcatgacatgaaacccagaaaccataaaagaaaagaatgataaaattgcccacgtaaagtaaaaagcttgcacacagaaaaacaccatacaggttacaagatgagcagcaaaatcagagaaaaaacattgcaattcaggacacacagaggctattgttcctaatatttaaaaataaaagtagtggattgtctacaaaaagatgaagacaagaatttcagaaaaccaaatactgcatgttttcacttacaagtggaagctaaacactgagtacacgtgtacacaaagaatggaaccataggccaggcaccgtggctcacgcctgtaatcccagtactttgcgaggccgaagcgggcggatcacctgaggtgaggagttcgagaccatcctggccaacatggtgaaacccagtctctactaaaaatacaaaaattagccgggcgtggtggtgggtgcctgtaatcccagctactcgggaggctgcggcagtagaatcgcttgaaccctggaggtggaccttgcagtgagccgagatcgcaccactgcactccagcctgggcaacagagtgagactccatctcaaaaaaaaaaaaaaggaatagaacaatagacactggggcctacttgagggaggagggtgaggatcaaaaacctgcctatcaggtactatgcttattacctgggtggtgaaataatctgtacaccaaaccccagtgacatgcaatttaccgatgtaacaaacctgcccatgtacccgctgaacctaaaataaaagttggaaaaaaatatagaaattttctttgtaatagccaaaaactgcaaacagcccaggtgtctattagtagaatgcataaacaaactcgggcatgttcatacaatgtaaaactactcatcaataaaaagtgatacttctcagcaatgaaaagaaactagctactgataccagctacaacatggatggatttcaagtgctttatgatgagagcaagaagccagacacaaaagtgtctatatatatatacagtatatatacgtatatatacacatatatacagtatatatatacatatacatgtatatatatactgtatatatactgtatatatatacacagtatatatatacatatatacagtgtatatatactgtgtatatatacatgtatatatactgtgtatatatacatgtatatatactgtgtatatatacatgtatatatactgtgtatatatacatgtatatatatgtatactgtatatatactgtatatatatatacacatatatacagtatatatatacagtatatactgtatatatacagtatatacgtgtatatatacatatatacagtatatatgtaaatatacatatatacagtatatatgtaaatatacatatatacatgtatatatatacactatatatatacatatatagtgtatatatacatatatacatgtatatatttactatatgattccatttatataaagtgccaaaacagtcaaaaataatctatgtggaaaaaatcaacaaagggatcccccgggctgcaggaattcgatggcgcgccgtcgacccaacgcgttgggagctctccggatccaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacttagattcatcgagagggacacgtcgactactaaccttcttctctttcctacagctgagatcaccggcgaaggagggccaccatgcatggggtggaaaatacttcccgtctgcttgtcgctgctgcttcctgttgtcttgattcaacaggtttcatctcaagagctctcctgcaaaggacgctgcttcgagtccttcgctcgagggagggagtgtgactgtgactcccagtgtaaacagtatggcaagtgctgtgcagattatgacagcttctgtgaagaagtaaaagataacaagaaaaacacccctaaaaagaaacccaatccagaaccgccggctgtggatgaagctggaagcgggctggataatggtgagttcaagctcaccccacctcctcctgaccctcctactactccacacagcaaagtcgccacatctcccaagactacagcagcaaaaccagtaactcctaaacccagtcttgcacctaattctgagacatccaaagaggcatctttagcatccaataaagagacaacagttgaaacgaaagagactactgcaacaaataaacaatcttctgccagtaaaaaaaagacgacttcagttaaagagacacggagtgcagagaaaacatctgataaagatgttgagcccacatctacaactcccaagaactcagctcccactactaccaagaagcctgttactacaaccaaggaatccaagttcctaccactcccacaggagcctgaacccacaactgccaaggaaccacctccaaccaccaagaagcctgaacccaccacccgcaaggagcctgaacccacaactcccaaggagcctgaacccaccacccccaaggagcctgaacccaccactcccaaggagcctgaacccacaactcccaaggaaccacctccaaccaccaagaagcctgaacccaccacccccaaggagcctggacccacaactcccaaggagcctgaacccaccaccaccaaggagcctgaacccaccaccaccaaggagcctgaatccaccactcgcaaggagcctgaacccaccactcccaaggagcctgaacccacaactcccaaggagcctgaacctacaaccctcaaggagcctgaacccacaactcccaaggagcctgaacccaccactcccaaggagcctgaacccacaactcccaaggagcctgaacccacaactcccaaggagcctgaacccaccacgcccaaggagcctgaacccacaactcccaaggagcctgaacccaccacgcccaaggagcctgaacccacaacccccaaggagcctgaacccacaactcccaagaaacctgaacccacaactcccaaggagcctgtacctacaacccccaaggagcctgaacccaccacccccaaggagcctgaacccaccacgcccaaggagcctgaacccacaactcgcaaggagcctgaacctacaactcccaaggagcctgaacctacaacccccaaggagcctgaacccacaactcccaagaagcctgaacccaccaccacatcacccaaaaccactactctgaaagcaacaactcttgcacccaaagtaactgctccagcagaagagattcagaacaaacctgaagaaacaactcctgcatcagaagattctgatgattctaaaacaactctaaaaccacagaagccaaccaaagcacccaagcctaccaaaaagccaaccaaagcacccaagaagcccacctctaccaaaaagccaaagacaccaaaaacaagaaaaccaaaaactacaccagctcctctaaagacgacttcagcaacacctgaactgaataccacccctctagaagtcatgctgccaaccaccaccatccctaaacaaactccaaaccctgaaacagctgaagtaaatccagatcatgaagatgcagatggaggtgaaggagaaaaacctctgattcccgggccccctgtgctattccccacagctattccaggcactgatcttttggccgggagactcaatcgaggcattaacatcaatcccatgccttcagatgagaccaatttatgcaatggtaagccagtggatggactgactacgctgcgcaatgggacattagttgcatttcgaggtcattatttctggatgctgaatccattcagaccaccatctccaccacgcagaattaccgaagtttggggtattccctctcctattgacacagtttttactagatgtaactgtgaaggaaaaactttcttctttaaggattctcagtactggcgcttcaccaatgatgtggtagatcctgggtatcctaaacaaattgtcaaaggctttggaggactaacagggaagatagtggctgctctttcaatagccaagtacaaggacaggcctgaatctgtgtacttcttcaagagaggtggcaacattcagcagtacacttataaacaggagccaatgaagaagtgcacagggaggcggcctgccatcaactactcggtgtacggggaggcagcacaggtcaggaggcgccgctttgagcgtgctgttggacctttccagacacacaccttcaggatccattactcggtgcccatgagagtttcttatcaagacaaaggtttcctccacaatgaagtcaaagtgagtacaatgtggagagggtttccaaatgttgttacttcagcgataacactgcctaacattaggaaacctgatggctatgattactacgccttttccaaagatcaatactataacattgatgtgcctacaagaacagcaagagcaattaccacacgttcagggcagaccctctccaaaatctggtacaactgtccttgaactgattgacaaagaagagtcatcaaaatgaaatgaagaaaacagtgatactttttgacattgaaatacattttattaataaagaatgttgaaatgagcatacctatttaaatacaaaaaaaaaaaaaaaaaaggccacatgtgctcgagctgcaggtcgcggccctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatcatttggggtaccgaattcctcgagtctagaggagcatgcgacgtcggcgcgcctaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttt

SEQ ID NO: 10 - DNA, 4215 bp, synthetic construct
  source 1..4215: mol_type = other DNA
  misc_feature 1..4215: note = human PRG4
atggcatggaaaacacttcccatttacctgttgttgctgctgtctgttttcgtgattcagcaagtttcatctcaagatttatcaagctgtgcagggagatgtggggaagggtattctagagatgccacctgcaactgtgattataactgtcaacactacatggagtgctgccctgatttcaagagagtctgcactgcggagctttcctgtaaaggccgctgctttgagtccttcgagagagggagggagtgtgactgcgacgcccaatgtaagaagtatgacaagtgctgtcccgattatgagagtttctgtgcagaagtgcataatcccacatcaccaccatcttcaaagaaagcacctccaccttcaggagcatctcaaaccatcaaatcaacaaccaaacgttcacccaaaccaccaaacaagaagaagactaagaaagttatagaatcagaggaaataacagaagaacattctgtttctgaaaatcaagagtcctcctcctcctcctcctcttcctcttcttcttcaacaattcggaaaatcaagtcttccaaaaattcagctgctaatagagaattacagaagaaactcaaagtaaaagataacaagaagaacagaactaaaaagaaacctacccccaaaccaccagttgtagatgaagctggaagtggattggacaatggtgacttcaaggtcacaactcctgacacgtctaccacccaacacaataaagtcagcacatctcccaagatcacaacagcaaaaccaataaatcccagacccagtcttccacctaattctgatacatctaaagagacgtctttgacagtgaataaagagacaacagttgaaactaaagaaactactacaacaaataaacagacttcaactgatggaaaagagaagactacttccgctaaagagacacaaagtatagagaaaacatctgctaaagatttagcacccacatctaaagtgctggctaaacctacacccaaagctgaaactacaaccaaaggccctgctctcaccactcccaaggagcccacgcccaccactcccaaggagcctgcatctaccacacccaaagagcccacacctaccaccatcaagtctgcacccaccacccccaaggagcctgcacccaccaccaccaagtctgcacccaccactcccaaggagcctgcacccaccaccaccaaggagcctgcacccaccactcccaaggagcctgcacccaccaccaccaaggagcctgcacccaccaccaccaagtctgcacccaccactcccaaggagcctgcacccaccacccccaagaagcctgccccaactacccccaaggagcctgcacccaccactcccaaggagcctacacccaccactcccaaggagcctgcacccaccaccaaggagcctgcacccaccactcccaaagagcctgcacccactgcccccaagaagcctgccccaactacccccaaggagcctgcacccaccactcccaaggagcctgcacccaccaccaccaaggagccttcacccaccactcccaaggagcctgcacccaccaccaccaagtctgcacccaccactaccaaggagcctgcacccaccactaccaagtctgcacccaccactcccaaggagccttcacccaccaccaccaaggagcctgcacccaccactcccaaggagcctgcacccaccacccccaagaagcctgccccaactacccccaaggagcctgcacccaccactcccaaggaacctgcacccaccaccaccaagaagcctgcacccaccactcccaaagagcctgccccaactacccccaaggagactgcacccaccacccccaagaagctcacgcccaccacccccgagaagctcgcacccaccacccctgagaagcccgcacccaccacccctgaggagctcgcacccaccacccctgaggagcccacacccaccacccctgaggagcctgctcccaccactcccaaggcagcggctcccaacacccctaaggagcctgctccaactacccctaaggagcctgctccaactacccctaaggagcctgctccaactacccctaaggagactgctccaactacccctaaagggactgctccaactaccctcaaggaacctgcacccactactcccaagaagcctgcccccaaggagcttgcacccaccaccaccaaggagcccacatccaccacctgtgacaagcccgctccaactacccctaaggggactgctccaactacccctaaggagcctgctccaactacccctaaggagcctgctccaactacccctaaggggactgctccaactaccctcaaggaacctgcacccactactcccaagaagcctgcccccaaggagcttgcacccaccaccaccaaggggcccacatccaccacctctgacaagcctgctccaactacacctaaggagactgctccaactacccccaaggagcctgcacccactacccccaagaagcctgctccaactactcctgagacacctcctccaaccacttcagaggtctctactccaactaccaccaaggagcctaccactatccacaaaagccctgatgaatcaactcctgagctttctgcagaacccacaccaaaagctcttgaaaacagtcccaaggaacctggtgtacctacaactaagactcctgcagcgactaaacctgaaatgactacaacagctaaagacaagacaacagaaagagacttacgtactacacctgaaactacaactgctgcacctaagatgacaaaagagacagcaactacaacagaaaaaactaccgaatccaaaataacagctacaaccacacaagtaacatctaccacaactcaagataccacaccattcaaaattactactcttaaaacaactactcttgcacccaaagtaactacaacaaaaaagacaattactaccactgagattatgaacaaacctgaagaaacagctaaaccaaaagacagagctactaattctaaagcgacaactcctaaacctcaaaagccaaccaaagcacccaaaaaacccacttctaccaaaaagccaaaaacaatgcctagagtgagaaaaccaaagacgacaccaactccccgcaagatgacatcaacaatgccagaattgaaccctacctcaagaatagcagaagccatgctccaaaccaccaccagacctaaccaaactccaaactccaaactagttgaagtaaatccaaagagtgaagatgcaggtggtgctgaaggagaaacacctcatatgcttctcaggccccatgtgttcatgcctgaagttactcccgacatggattacttaccgagagtacccaatcaaggcattatcatcaatcccatgctttccgatgagaccaatatatgcaatggtaagccagtagatggactgactactttgcgcaatgggacattagttgcattccgaggtcattatttctggatgctaagtccattcagtccaccatctccagctcgcagaattactgaagtttggggtattccttcccccattgatactgtttttactaggtgcaactgtgaaggaaaaactttcttctttaaggattctcagtactggcgttttaccaatgatataaaagatgcagggtaccccaaaccaattttcaaaggatttggaggactaactggacaaatagtggcagcgctttcaacagctaaatataagaactggcctgaatctgtgtattttttcaagagaggtggcagcattcagcagtatatttataaacaggaacctgtacagaagtgccctggaagaaggcctgctctaaattatccagtgtatggagaaacgacacaggttaggagacgtcgctttgaacgtgctataggaccttctcaaacacacaccatcagaattcaatattcacctgccagactggcttatcaagacaaaggtgtccttcataatgaagttaaagtgagtatactgtggagaggacttccaaatgtggttacctcagctatatcactgcccaacatcagaaaacctgacggctatgattactatgccttttctaaagatcaatactataacattgatgtgcctagtagaacagcaagagcaattactactcgttctgggcagaccttatccaaagtctggtacaactgtccttag

SEQ ID NO: 11 - DNA, 2894 bp, synthetic construct
  source 1..2894: mol_type = other DNA
  misc_feature 1..2894: note = murine PRG4
atggggtggaaaatacttcccgtctgcttgtcgctgctgcttcctgttgtcttgattcaacaggtttcatctcaagagctctcctgcaaaggacgctgcttcgagtccttcgctcgagggagggagtgtgactgtgactcccagtgtaaacagtatggcaagtgctgtgcagattatgacagcttctgtgaagaagtaaaagataacaagaaaaacacccctaaaaagaaacccaatccagaaccgccggctgtggatgaagctggaagcgggctggataatggtgagttcaagctcaccccacctcctcctgaccctcctactactccacacagcaaagtcgccacatctcccaagactacagcagcaaaaccagtaactcctaaacccagtcttgcacctaattctgagacatccaaagaggcatctttagcatccaataaagagacaacagttgaaacgaaagagactactgcaacaaataaacaatcttctgccagtaaaaaaaagacgacttcagttaaagagacacggagtgcagagaaaacatctgataaagatgttgagcccacatctacaactcccaagaactcagctcccactactaccaagaagcctgttactacaaccaaggaatccaagttcctaccactcccacaggagcctgaacccacaactgccaaggaaccacctccaaccaccaagaagcctgaacccaccacccgcaaggagcctgaacccacaactcccaaggagcctgaacccaccacccccaaggagcctgaacccaccactcccaaggagcctgaacccacaactcccaaggaaccacctccaaccaccaagaagcctgaacccaccacccccaaggagcctggacccacaactcccaaggagcctgaacccaccaccaccaaggagcctgaacccaccaccaccaaggagcctgaatccaccactcgcaaggagcctgaacccaccactcccaaggagcctgaacccacaactcccaaggagcctgaacctacaaccctcaaggagcctgaacccacaactcccaaggagcctgaacccaccactcccaaggagcctgaacccacaactcccaaggagcctgaacccacaactcccaaggagcctgaacccaccacgcccaaggagcctgaacccacaactcccaaggagcctgaacccaccacgcccaaggagcctgaacccacaacccccaaggagcctgaacccacaactcccaagaaacctgaacccacaactcccaaggagcctgtacctacaacccccaaggagcctgaacccaccacccccaaggagcctgaacccaccacgcccaaggagcctgaacccacaactcgcaaggagcctgaacctacaactcccaaggagcctgaacctacaacccccaaggagcctgaacccacaactcccaagaagcctgaacccaccaccacatcacccaaaaccactactctgaaagcaacaactcttgcacccaaagtaactgctccagcagaagagattcagaacaaacctgaagaaacaactcctgcatcagaagattctgatgattctaaaacaactctaaaaccacagaagccaaccaaagcacccaagcctaccaaaaagccaaccaaagcacccaagaagcccacctctaccaaaaagccaaagacaccaaaaacaagaaaaccaaaaactacaccagctcctctaaagacgacttcagcaacacctgaactgaataccacccctctagaagtcatgctgccaaccaccaccatccctaaacaaactccaaaccctgaaacagctgaagtaaatccagatcatgaagatgcagatggaggtgaaggagaaaaacctctgattcccgggccccctgtgctattccccacagctattccaggcactgatcttttggccgggagactcaatcgaggcattaacatcaatcccatgccttcagatgagaccaatttatgcaatggtaagccagtggatggactgactacgctgcgcaatgggacattagttgcatttcgaggtcattatttctggatgctgaatccattcagaccaccatctccaccacgcagaattaccgaagtttggggtattccctctcctattgacacagtttttactagatgtaactgtgaaggaaaaactttcttctttaaggattctcagtactggcgcttcaccaatgatgtggtagatcctgggtatcctaaacaaattgtcaaaggctttggaggactaacagggaagatagtggctgctctttcaatagccaagtacaaggacaggcctgaatctgtgtacttcttcaagagaggtggcaacattcagcagtacacttataaacaggagccaatgaagaagtgcacagggaggcggcctgccatcaactactcggtgtacggggaggcagcacaggtcaggaggcgccgctttgagcgtgctgttggacctttccagacacacaccttcaggatccattactcggtgcccatgagagtttcttatcaagacaaaggtttcctccacaatgaagtcaaagtgagtacaatgtggagagggtttccaaatgttgttacttcagcgataacactgcctaacattaggaaacctgatggctatgattactacgccttttccaaagatcaatactataacattgatgtgcctacaagaacagcaagagcaattaccacacgttcagggcagaccctctccaaaatctggtacaactgtccttgaactgattgacaaagaagagtcatcaaaatgaaatgaagaaaacagtgatactttttgacattgaaatacattttattaataaagaatgttgaaatgagcatacctatttaaataca

SEQ ID NO: 12 - protein, 1404 aa, Homo sapiens
  UNSURE 1..1404: note = human PRG4
MAWKTLPIYLLLLLSVFVIQQVSSQDLSSCAGRCGEGYSRDATCNCDYNCQHYMECCPDFKRVCTAELSCKGRCFESFERGRECDCDAQCKKYDKCCPDYESFCAEVHNPTSPPSSKKAPPPSGASQTIKSTTKRSPKPPNKKKTKKVIESEEITEEHSVSENQESSSSSSSSSSSSTIRKIKSSKNSAANRELQKKLKVKDNKKNRTKKKPTPKPPVVDEAGSGLDNGDFKVTTPDTSTTQHNKVSTSPKITTAKPINPRPSLPPNSDTSKETSLTVNKETTVETKETTTTNKQTSTDGKEKTTSAKETQSIEKTSAKDLAPTSKVLAKPTPKAETTTKGPALTTPKEPTPTTPKEPASTTPKEPTPTTIKSAPTTPKEPAPTTTKSAPTTPKEPAPTTTKEPAPTTPKEPAPTTTKEPAPTTTKSAPTTPKEPAPTTPKKPAPTTPKEPAPTTPKEPTPTTPKEPAPTTKEPAPTTPKEPAPTAPKKPAPTTPKEPAPTTPKEPAPTTTKEPSPTTPKEPAPTTTKSAPTTTKEPAPTTTKSAPTTPKEPSPTTTKEPAPTTPKEPAPTTPKKPAPTTPKEPAPTTPKEPAPTTTKKPAPTTPKEPAPTTPKETAPTTPKKLTPTTPEKLAPTTPEKPAPTTPEELAPTTPEEPTPTTPEEPAPTTPKAAAPNTPKEPAPTTPKEPAPTTPKEPAPTTPKETAPTTPKGTAPTTLKEPAPTTPKKPAPKELAPTTTKEPTSTTCDKPAPTTPKGTAPTTPKEPAPTTPKEPAPTTPKGTAPTTLKEPAPTTPKKPAPKELAPTTTKGPTSTTSDKPAPTTPKETAPTTPKEPAPTTPKKPAPTTPETPPPTTSEVSTPTTTKEPTTIHKSPDESTPELSAEPTPKALENSPKEPGVPTTKTPAATKPEMTTTAKDKTTERDLRTTPETTTAAPKMTKETATTTEKTTESKITATTTQVTSTTTQDTTPFKITTLKTTTLAPKVTTTKKTITTTEIMNKPEETAKPKDRATNSKATTPKPQKPTKAPKKPTSTKKPKTMPRVRKPKTTPTPRKMTSTMPELNPTSRIAEAMLQTTTRPNQTPNSKLVEVNPKSEDAGGAEGETPHMLLRPHVFMPEVTPDMDYLPRVPNQGIIINPMLSDETNICNGKPVDGLTTLRNGTLVAFRGHYFWMLSPFSPPSPARRITEVWGIPSPIDTVFTRCNCEGKTFFFKDSQYWRFTNDIKDAGYPKPIFKGFGGLTGQIVAALSTAKYKNWPESVYFFKRGGSIQQYIYKQEPVQKCPGRRPALNYPVYGETTQVRRRRFERAIGPSQTHTIRIQYSPARLAYQDKGVLHNEVKVSILWRGLPNVVTSAISLPNIRKPDGYDYYAFSKDQYYNIDVPSRTARAITTRSGQTLSKVWYNCP

SEQ ID NO: 13 - protein, 925 aa, Mus musculus
  UNSURE 1..925: note = murine PRG4
MGWKILPVCLSLLLPVVLIQQVSSQELSCKGRCFESFARGRECDCDSQCKQYGKCCADYDSFCEEVKDNKKNTPKKKPNPEPPAVDEAGSGLDNGEFKLTPPPPDPPTTPHSKVATSPKTTAAKPVTPKPSLAPNSETSKEASLASNKETTVETKETTATNKQSSASKKKTTSVKETRSAEKTSDKDVEPTSTTPKNSAPTTTKKPVTTTKESKFLPLPQEPEPTTAKEPPPTTKKPEPTTRKEPEPTTPKEPEPTTPKEPEPTTPKEPEPTTPKEPPPTTKKPEPTTPKEPGPTTPKEPEPTTTKEPEPTTTKEPESTTRKEPEPTTPKEPEPTTPKEPEPTTLKEPEPTTPKEPEPTTPKEPEPTTPKEPEPTTPKEPEPTTPKEPEPTTPKEPEPTTPKEPEPTTPKEPEPTTPKKPEPTTPKEPVPTTPKEPEPTTPKEPEPTTPKEPEPTTRKEPEPTTPKEPEPTTPKEPEPTTPKKPEPTTTSPKTTTLKATTLAPKVTAPAEEIQNKPEETTPASEDSDDSKTTLKPQKPTKAPKPTKKPTKAPKKPTSTKKPKTPKTRKPKTTPAPLKTTSATPELNTTPLEVMLPTTTIPKQTPNPETAEVNPDHEDADGGEGEKPLIPGPPVLFPTAIPGTDLLAGRLNRGININPMPSDETNLCNGKPVDGLTTLRNGTLVAFRGHYFWMLNPFRPPSPPRRITEVWGIPSPIDTVFTRCNCEGKTFFFKDSQYWRFTNDVVDPGYPKQIVKGFGGLTGKIVAALSIAKYKDRPESVYFFKRGGNIQQYTYKQEPMKKCTGRRPAINYSVYGEAAQVRRRRFERAVGPFQTHTFRIHYSVPMRVSYQDKGFLHNEVKVSTMWRGFPNVVTSAITLPNIRKPDGYDYYAFSKDQYYNIDVPTRTARAITTRSGQTLSKIWYNCP

SEQ ID NO: 14 - DNA, 534 bp, synthetic construct
  source 1..534: mol_type = other DNA
  misc_feature 1..534: note = equine IL-1Ra
atggaaatccgcaggcgttctgtcagacacctaatctctctcctccttttcttgctctactcagagacagcctgccaccctttggggaagagaccctgcaagatgcaagccttcagaatctgggatgttaaccagaagaccttctacatgaggaataaccaactagttgctggatacttgcaagaatcaaatactaaattacaagagaagatagatgtggtgcccattgagcctgatgctctattcctgggactccatgggaggaagctgtgcctggcctgtgtcaagtctggtgatgagattaggttccaattggaggcagttaacatcactgacctgagcaagaacaaggaggagaacaagcgcttcaccttcatccgctcaaacagtggccccaccaccagcttcgagtctgccgcctgccctggctggttcctctgcacggcgcaggaggcagaccggcccgtcagcctcaccaacaagcccaaagagtccttcatggtcaccaagttctacctccaggaggaccagtag

SEQ ID NO: 15 - protein, 178 aa, Mus musculus
  UNSURE 1..178: note = murine IL-1Ra
MEICWGPYSHLISLLLILLFHSEAACRPSGKRPCKMQAFRIWDTNQKTFYLRNNQLIAGYLQGPNIKLEEKIDMVPIDLHSVFLGIHGGKLCLSCAKSGDDIKLQLEEVNITDLSKNKEEDKRFTFIRSEKGPTTSFESAACPGWFLCTTLEADRPVSLTNTPEEPLIVTKFYFQEDQ

SEQ ID NO: 16 - protein, 177 aa, Equus caballus
  UNSURE 1..177: note = equine IL-1Ra
MEIRRRSVRHLISLLLFLLYSETACHPLGKRPCKMQAFRIWDVNQKTFYMRNNQLVAGYLQESNTKLQEKIDVVPIEPDALFLGLHGRKLCLACVKSGDEIRFQLEAVNITDLSKNKEENKRFTFIRSNSGPTTSFESAACPGWFLCTAQEADRPVSLTNKPKESFMVTKFYLQEDQ

SEQ ID NO: 17 - DNA, 4425 bp, synthetic construct
  source 1..4425: mol_type = other DNA
  misc_feature 1..4425: note = Equine PRG4
atggagtggaaaatacttcccatttacttgttgctgctgctttctattttctccattcaggaagtttcttctcaagatttatcaagctgtgcagggagatgtggggaagggtattctagagatgccacctgcaactgtgattttaactgccaatactacatggagtgctgccctgatttcaagaaagtctgcacctcggagctttcctgtaaaggtcgctgcttcgagtcctttgaaagagggagggagtgtgactgtgacgcggactgcaagaagtacggcaagtgctgttccgattatgagagtttctgtgaagaagtgcataatcccacatcaccaccatcttcaaagactgcgcctccgcctccaggagcatctcaaaccatcaaatcaacagccaaacgttcacccaaatcaaacaagaagaagactaagaaagttatagaatcagaggaaataatagaagaacattctgtttctgaaaatcaagagtcttcctcctcctcttcctcttcctcttcaactattcggaaagtcaagtcttccaaaaattcagctgctaatagagaattaaagaagaaacccaaagtaaaagacagcaagaagaaaagaactcctaaaaagaaacctactccagaaccaccagttatagatgaagctggaagtggactggacaatggtgatttcatgctcatcccaactcctaagattcctaccacccaacgcaacaaagttactacatctcccaagattacaacagtaaaaccaataaatcctaaacccagtcttccacctaattctgatacatccaaagagacaacttcaacacccaataaggagacaacagttgaaactaaagagacagagataacaaataaagagacttcaactagtgcaaatgagaagactacttcagctagaaagagtacagagaaaacatctgataaagattttgcacccgcatctgaagttcctgctaaatctacacccaaagctgaaactacaaccaaatccccttctctcaccaccaccaaggggcctgctccaactactgctaaggggcctgctcccactaccaccaagaagcctacttccaccaccaccgaggggcctgcacccaccacccccgaggagcccgctcccaccacccccgaggagcctacttctaccacgaccaaggagcccgcacccaccaccctcgaggagaccgctcccaccacccccgaggagacggctcccaccacccccgagaagcccgctcccaccacccccaaggagcccacttctaccacgaccaaggagcccgcacccaccacccccgaggagcccgctcccaccacccccaaggagcctgcacccaccacacccaaggagacggctcccaccaccaccaaggagcccgcacccaccaccctcaaggagcctgcacccaccacacccaaggagacggctcccaccaccaccaaggagctggcacccaccaccctcaaggagcctgctcccaccacccccgaggagtccgctcccaccacacccgaggagacggctcccaccagccccgaggagcccgctcccaccacccccgaggagcccgcacctaccacgaccaaggagccggcacccaccaccctcaaggagcccgctcccaccaccgccgaggagcccgctcccaccacccccgaagagcccgcacctaccacgaccaaggagccggcacccaccaccctcaaggagcccgcacccaccaccgccgaggagcccgctcccaccaccctcaaggagcccgctcccaccaccgccgaggagccggctcccaccacccccgaggagcccgcacctaccacacccaaggagcccgcacccaccaccctcaaggagcccactcccaccacccccaaggagcccgcacccaccaccaccaaggagcccgcacccaccaccctcaaggagcccgctcccaccacccccaaggagcccgcacccaccaccaccaaggagccctcttccaccacacccaaggagatggctcccaccacccccgaggagcccgcacctaccacacccaaggagcccgcacccaccaccctcaaggagcccgctcccaccaccgccgaggagcccgctcccaccaccgccgaggagcccgctcccaccacccctgaggagcccgcacctaccacgaccaaggagcccgcacccaccaccctcaaggagcctgcacccaccacccccaaggagcctgcacccaccacccccaaggagccagctcccaccacccccaaggagcctgcacccactacccccaaggagcccgctccccctactcccaaggagcccgttcccaccagcccaaaggagcctgcacccaccacccctaaggagcccgctcccactacctccaaggagcctgctcccaccacccccaaggagcccacacccaccacccctaaggagccagctcccactacctccaagaagcctgcacccactagccctaaggagcccatacccaccatccctaaggagtctgcatccaccacccctgaggaacccgcacccaccccccccgaggagcctgctctcactacccccaagaaacaggctcccaccaccaccacggagcctatactcaccacccctaaggagcctgcacctatcaccacgaaggaacctgctctggccacttccaaaaagcctccgcccagccccacccccaaggagcccgctccaacttctcctgagacacctgctccaaccacctcagaggcctctactacaactaccaccatgaagcctcccactactcccaagaatcttgctgaatcaactcctgagtttcctgcagaacccacaccaaaggctcttgaaaacagtcccaaggagccggctgtacctacgaccaaggctcccgaagtgaccaaacctgaagtaactacaacagctaaagacaaggtaacaggaaaagacatacatactatacctgaaattacaaccgctgcacctaaaattacaacagagacagcaactacaacagaagaaaagaccactgaatccaaagtaacaagtacaatcatgcaagtaacgtctacaactgaagatactacgacatcatccaaaattactcccaaagctacaactcttgcacccaaagtaatgactgccacaaaaacaactacaacccaagaaactataaataaactggaagaaacaacagctataccaaaagatacagctactcattctaaagtgacaactcctaaacctaaaaagccaaccaaagcacccagaaagcccacttctaccaaaaagcccaaaacaccacgtaagagaaaaccaaagactacaccaattcccccaaagataactacaccaacaacgcccaaatcgaaccctaccactttagcggaagccatgctccaaaccaccaccagccctaaccaaactcctaactcagcaatgattgaagtaaatccaaagaatgaagatgcagatgctgctgaaggagaaaaacctctcgtgattctcaggccccatgtgttaactcctatagttattccaggtcctgatttcttagtgagaggacccaatctgggcattggcatcaaccccatgctttcagatgagactaatttatgcaatggtaaaccagtggacggactgaccactttgcgcaacgggacgttggttgcatttcgaggtcattatttctggatgctacgtccattcagcccaccatctccacctcgcagaattactgaagtttggggtattccctcccccattgatactgttttcaccagatgcaactgcgaaggaaaaactttcttcttcaaggattctcaatactggcgtttcaccaatgatataaaagatgcagggtatcccaaactaatttcaaaaggatttggaggactaagtggaaaaatagtggcagctctttcaatagccacatacaagaacagacctgaatctgtgtattttttcaagagaggtggcaggattcagcagtatatttataaacaggaacccatcagaaagtgcccgggaaggcggcctgctatccattattcggtgtatggggaagcaccgcagattcggagacgccgcttcgaacgggctataggaccttctcaaacacacaccatccgaattcactattcacccgtcagagtctcttatcaagacaaagtgccctccacagatttcctccataatgaagttaaagtgagtacgctctggagaggacttcccgatacggttacttccgctatatcactgcccaacctcagaaaacccgatggctacgattactatgccttttctaaggatcaatactataacatcgatgtgcctagtagaacagcaagagcaattactactcgctctgggcagaccttatccaaagtctggtacaactgtccttag

SEQ ID NO: 18 - DNA, 4467 bp, synthetic construct
  source 1..4467: mol_type = other DNA
  misc_feature 1..4467: note = Canis lupus PRG4
atgcagtggaaaatacttcccatatacttgctgctcctctctgttttcttgattcagcaagtttcttctcaagatttaccaagctgtgcagggagatgtggggaagggtattctagagatgccatctgcaactgtgattataactgtcaacactacatggagtgctgccctgatttcaagaaagcctgcactgtggagctttcctgtaaaggtcgctgcttcgagtcctttgcacgagggagggagtgtgactgtgactcagactgtaagaagtatggcaagtgctgtcccgattatgaggatttttgtggaagagtgcataatcccacatcacctccatcttcaaagactgcacctccatctccaggagcatctcaaaccatcaaatcaacagccaaacgttcacccaaagcaccaaataagaagaagactaagaaagttatagaatcagaggaaataacagaagaacattctgtttctgaaaaccaagagtcttcttcctcctcttcctcttcctcttcaactattcggaaaatcaagtcttccaaaaattcagcagctaataaagaattaaagaagaaacccaaagtaaaagataacaagaaggaaagaactcctaaaaagaaacctccgccagaaccaccagttgtagatgaagctggaagtggactggacaatggtgacatcaagctcaccccaactcctgacattcctaccacccaacgcaataaggttaccacatctcccaagtttacaacaggcaaaccaataaatcctaaacctagtcttccacctaatactgatacatccaaagagacctcttcaacacctaataaggagacaacagtgaaaagtaaagagactttagcaaacaaagagacttcaagtaaagcaaaagagaagattacttcagccaaagagacacgaagtgcagagaaaacacctgctaaagattttgtacccacaaccaaagctcctgttaaatctacacccaaagctgaaagtacaaccaaatctcctgctcccaccaccaccaaggagcccactcctaccaccaccaagaagcctgcacccactacccccaagaaacctgctcccactactcccaaggagcctgtacccactaccaccaaggggccacccaccacgcccaagaaacctgaacccaccactcccaaggatcctgctcccaccaccaccaaggagcccactcccaccacccccaagaagcctgctcccactactcccaaggagcctgtacccattaccaccaaggagcctgaacccaccacccccaaggagcctgctcccaccactcccaaggagcctgtacctactaccaccaaggagccacccaccacccccaagaagcctgaacccaccactcccaaggagcctgctcccaccactcccaaggagcctgtacccactaccaccaaggagcctgaacccaccactcccaaggagcctgcacccaccacccccaaggagcctgctcccactactcccaaggagcctgtacctactaccaccaaggagccacccaccacccccaagaagcctgaacccaccactcccaaggagcctgctcccaccactcccaaggagcctgtacccactaccaccaaggagcctgaacccaccactcccaaggagcttgcacccaccacccccaaggagcctgctcccactactcccaaggagcctgtacctactaccaccaaggagccacccaccacccccaagaagcctgaacccaccactcccaaggagcctgctcccaccactcccaaggagcctgtacccactaccaccaaggagcctgaacccaccactcccaaggagcctgcacccaccacccccaaggagcctgctcccactactcccaaggagcctgtacctactaccaccaaggagccacccaccacccccaagaagcctgaacccaccactcccaaggagcctgctcccaccactcccaaggagcctgtacccactaccaccaaggagcctgaacccaccactcccaaggagcttgcacccaccacccccaaggagcctgctcccactactcccaaggagcctgtacctactaccaccaaggagccacccaccacccccaagaagcctgaacccaccactcccaaggagcctgcacccaccacccccaaggagcctgctcccactactcccaaggagccacctaccacccccaagaagcctgaacccaccactcccaaagaggctgctcccaccaccaagaaaccagctgccaccactcccaaggagcctgcacccactatcactaaggagcctgcaccaactactcccaacaagcctgaacccaccactcccaaagagcctgtgcccacaacccccaaggagcctgaacccactccccctaaggaacctgctcctaccaccaccaaggaccctgcacctaccagtcccaaggaacctactcccaccgcccccaaggagcctgtacctactgcccccaaggagcctgaacccatggcccccaagaagcctgtacccactgcccccaagcagcctacacccaccacccccaaggagccttcacccactgtccccaaggagcctgaacctatggcccccaaggagcctgtacccacagctcccaagaaacctgcacccaccgcccccaaggaccctgcacccaccgcccccaaggagcctgaacccactgcccccaataaggaatctgcacccaccacatccaaggaacaggttcccatcaccaccaaggagcccacacccaaactcccgaaggagcctgctccagcctctcttgagacgcctgctccaaccacctcagacgcctttactacaactacgactatggagcctcccactactcccaagaaccctgctgagtcaactcctaagtttcctgcagaacccacaccaaagcctcttgaaaacagtcccaaagaaccagttgtacctataaccaaggctcctgaagtgaccaaacctgaaatgactacaacagctaaagataaaacaacagaaaaagacataatacctgaaattacaactgctgtacctaagattacaacccaggagacagcaactccaacagaagaaacgaccactgagtccaaaacaagtacaaccacacaagtaacatctaccacatcatccaaaaacactcctaaagcaacaactctcgcacccaaagtaatgactgcaacacaaaagacaactacaactgaagagactatgaataaacctgaagaaaccacagctgtgccaaaggatacagctacgagtactaaagtctcaactcctagaccccgaaagccaaccaaagcaccaaagaagcccacttctaccaaaaagccaaacacaatacctaaaagaaaaaaaccaaagactacaccaactcccccaaagatgactacatcgacaatgcccaaattacaccctacctcttcagtggaagccatgctccaaactaccaccagccccaaccaaagacctaactcagaaatagttgaagtaaatccaaatgaagatacagatgctgctggaaaaaaacctcacatgttccccaggccccctgtgttaactcctatatttatcccagggactgatatcttagtgagaggatccaatcaagacattgccatcaatcccatgctttcagatgagactaatttatgcaacggtaagccagtagatggactgactactttgcgcaatggaaccatggtcgcatttcgaggtcattatttctggatgctgagtccatccaatccaccatctccacctcgtaaaattactgaagtttggggtattccctcccccattgatactgtttttactaggtgcaactgtgaaggaaaaactttcttctttaagggttcccagtactggcgtttcaccaatgatataaaagatgcagggtatcccaaacaaattgtaaaaggatttggaggactaaatggaagaatagtggcagctctctcaatagctaaatacaaggacagacctgaatctgtgtattttttcaagagaggtggcagcgttcagcagtacacttataaacaggaacccatcaaaaagtgcactggaagaaggcccgctatcaattacccagtgtatggagaaacaacacaggttagaagacgtcgctttgaacgcgccataggaccttctcaaacacacaccatcagaattcactattcacccatcagagtctcttatcaagataaaggtttcctccataatgaagtcaaaatgagttcacagtggagaggatttccaaatgtggttacttcagctatagcactgcccaacatcagaaaacctgatggctatgattactacgccttttctaggaatcaatactataacattgatgtacccagcagaacagcaagagttgttactactcgttttgggaggaccttatccaatatctggtacaactgtccttag

SEQ ID NO: 19 - DNA, 4500 bp, synthetic construct
  source 1..4500: mol_type = other DNA
  misc_feature 1..4500: note = Felis catus PRG4
atggagtggagaatacttcccatgtacttgctgctgctttctgttttcttgattcagcaagtttcttctcaagatttaccaagctgtgcagggagatgtggggaagggtattctagagatgccacctgcaactgtgattataactgtcaacactacatggagtgctgccctgatttcaagagagtctgcactgtggagctttcctgtaaaggccgctgctttgagtcctttgcaagagggagggagtgcgactgcgactcggaatgtaagaagtatggcaagtgctgttccgattatgagaatttttgtggaaaagtgcataatcccccatcaccatcttcaaagactgtacctccagctccaggagcatctcaaaccatcaaatcaacaaccaaacgttcacccaaatcaccaaacaagaagaagactaagaaagttatagaatcagaggaaataacagaagaacattctgtttctgaaaatcaagagtcttcttcctcctcttcctcttcctcttcaactattcggaaagtcaagtcttccaaaaattcagctgctaataaagaattaaagaagaaacccaaagtaaaagataacaagaaggaaaaaactcctaaaaagaaacctaaccctgaaccaccagttgtagatgaagatggaagtggactggacaatggtgatgtcaagctcaccccgactcctgacattcctaccacccaacgcaataaagttaccacacctcccaagattacaacagtcaaaccgacaaatccaaaacccagtcttccacctaattctgatacacccaaagagacatcttcaacaacgagtgaggagacagcagttaaaactaaagagactcctataacgaataaacagacttcaaatagagaaaaagagaagactacttcacctaaagagaaacaaagtgcagagaaaatacctgttaaagattttatacccacatccaaagctcctagtacatctacaccaaaagctgaaactacaaccaaatctcctgctcccaccaccaccaagaaacctgttcccactacccccaagaagcctgcacccaccaccaaagagcctgcacccaccacccccaagaagcctgtacccaccaccaaagagcctgcacccaccacccccaagaagcctgcacccaccaccaaagagcctgcaccctccacccccaagaagcctgcacccaccaccaaagagcctgcaccctccacccccaagaagcctgcacccaccaccaaagagcctgcacccaccacccccaagaagcctgcacccacgaccaaagagcctgcacccaccacccccaagaagcctgcacccacgaccaaagagcctgcacccaccacccccaagaagcctgcacccacgaccaaagagcctgcacccaccacccccaagaagcctgcacccacgaccaaagagcctgcacccaccacccccaagaagcctgcacccacgaccaaagagcctgcacccaccacccccaagaagcttgcacccacgaccaaagagcctgcacccaccacccccaagaagtctgctcccaccactcccaaggagcctgcatccactaccaccaaagaacctccacccacagcccccacggagcctgcactcaccactccaaaggagcctacacccactgcccccaaggaggctgcacccaccacccccaacaagcctgcttctattaccaccaaggaacctgcacccacagcccccaaggagcctgcacctaccacccccaataagcctgctcctactaccaccaaggaacctgcacccacggccctcaaggagcctgcacctaccacccccaaggaacctgcacccatggcccccacggagcctgcacccaccactcccaaggagcctgcatccacagcccccaaggaacctgctcccagcattcccaaggagcctgcacccaccactcccaaggagcctgcacccacagcccccaaggaacctgctcccagcattcccaaagaacctgcacccacagcccccaaggaacctgctcccagcattcccaaggagcctgcacccaccactcccaaggagcctgcacccacagcccccaaggaacctgctcccagcattcccaaggagcctgcacccatggcccccacagagcctgcacccaccactcccaaggagcctgcacccaccactcccaaggagcctgcacccacagcccccaaggaaactgctcccagcattcccaaggagcctgcacccacagcccccaaggaacctgctcccagcattcccaaggagcctgcacccacagcccccaaggaaactgctcccagcattcccaaggagcctgcacccacagcccccaaggaacctgctcccagcattcccaaggagcctgcacccacagcccccaaggaacctgctcccagcattcccaaagaacctgcacccacagcccccaaggaacctgctcccagcattcccaaggagcctgcacccatggcccccacagagcctgcacccaccactcccaaggagcctgcacccaccactcccaaggagcctgcacccacagcccccaaggaacctgctccgagcattcccaaggagcctgcacccatggagcctgcacccaccacccccaaggagcctgcacccaccgtccccaaggagcctgcacccaccgctcccaaggagcctgcacccaccgctcccaaggagcctgcacccacagcccccaaagaacctgctccgagcattcccaaggagcctgcacccatggtccccaaggagactgctcccaccaccaccaaggagcccatccccaaacttctcagggagcccactccagcttctcttgagacacctgctccaaccaactcagagggctctactacaaccaccatggagcctcccactactcccgaaaaccctgctgaatcaactcctgagtttcccaaagaacccacaccaaaggctcttgaaaacagtcccaaagaacctgttatacctgtaactaaggctcctggagtgaccacacctgaaatcactacaacagctaaagacaaaacaacagaaaaagacatacacatgacacctggaattatagccgctgtacctacatcaactccgacaaaagaaaagaccactgattccaaaacaagaacaaccacacaagtaacatcagccacatcgtccaaaaatactcctcaagccacaactcttgcacccaaagtaatgactacaacaaaaaaggcaactacatctgaagagactatgaataaacccgaagaaaccacagctgcgccaaaagacagagctaatcctaaagtgtcaactcctaaatcccaaaagccaaccaaagcaccaaaaaagcccacttctaccaaaaagccaaacacgagacctaaagggagaaaaccaaagactacaccaactcccccaaggatgactacatcgacagtgcccaaattacaccccacctcttccacaaaagccattctccaaactaccaccagccccaaccaaacacctaactcagaaatagttgaagtaaatccaaataaggatggagatgctgcaggagaaaaacctcacatgatccccaggccccctgtgttaactcctatatttatcccaggcacggatatcttagtgagaggatccaatcaagacattgccatcaactccatgctttcagatgagactaatttatgcaacggtaagccagtagatgggctgactgctttgcgcaatggaacgttagttgcatttcgaggtcattatttctggatgctaagtccattcagtccaccatctccagctcgtaaaattactgaagtttggggtattccctcccccattgatactgtttttactaggtgcaactgtgaaggaaaaactttcttctttaaggattctcagtactggcgtttcaccaatgacataaaagatgcagggtatcccaaacaaattgtaaaaggatttggaggactaaatggaaaaatagtggcagctctctccatagctaaatacaaggaaagacctgaatctgtgtattttttcaagagaggtggcggcattcagcagtatatttataaacaggaacccgtcagaaagtgcactggtagaaggcctgctatcaattattcagtgtatggagaaacaacacaggttaggagacgtcgctttgaacgcgccataggaccttctcaaacacacaccatcagaattcgctattcacccatcagagtctcttatcaagacaaaggtttcctccataatgaagttaaaatgagtttatactggagaggatttccaaatgtggttacttcagctatagcactgcccaacaccagaaaacctgatggctatgattattatgccttttctaaagatcaatactataacatcgatgaaccaagtagaacagcaagagtcattactactcgttctgggcggaccttgtccaatgtctggtacaactgtccttag